(19) United States
(12) Patent Application Publication
(10) Pub. No.: US 2020/0054643 A1
(43) Pub. Date: Feb. 20, 2020

(54) FUSED HETEROCYCLIC COMPOUNDS AS SELECTIVE BMP INHIBITORS

(71) Applicant: Vanderbilt University, Nashville, TN (US)

(72) Inventors: Corey R. Hopkins, Bennington, NE (US); Charles C. Hong, Nolensville, TN (US); Craig W. Lindsley, Brentwood, TN (US); Darren W. Engers, Nashville, TN (US)

(21) Appl. No.: 16/478,772

(22) PCT Filed: Jan. 18, 2018
(86) PCT No.: PCT/US2018/014239

§ 371 Date: Jul. 17, 2019

Related U.S. Application Data
(60) Provisional application No. 62/447,830, filed on Jan. 18, 2017.

(51) Int. Cl. A61K 31/5377; A61K 31/4725; A61K 31/437; A61K 31/4709; A61K 31/498; A61K 31/496; A61K 31/506; A61K 31/519; A61K 31/4985

(52) U.S. Cl. CPC A61K 31/5377; A61K 31/4725; A61K 31/437; A61K 31/4709; A61K 31/4985; A61K 31/496; A61K 31/506; A61K 31/519; A61K 31/498

(57) ABSTRACT

The present invention provides small molecule inhibitors of BMP signaling that are useful for treating diseases or conditions associated with BMP signaling, including cancers of the central nervous system.

### RELATED APPLICATION

This application claims the benefit of priority to U.S. Provisional Patent Application Ser. No. 62/447,830, filed Jan. 18, 2017, which is hereby incorporated herein by reference in its entirety.

### BACKGROUND

Signaling involving the Transforming Growth Factor β (TGF-β) superfamily of ligands is central to a wide range of cellular processes, including cell growth, differentiation, and apoptosis. TGF-β signaling involves binding of a TGF-β ligand to a type II receptor (a serine/threonine kinase), which recruits and phosphorylates a type I receptor. The type I receptor then phosphorylates a receptor-regulated SMAD (R-SMAD; e.g., SMAD1, SMAD2, SMAD3, SMAD5, SMAD8 or SMAD9), which binds to SMAD4, and the SMAD complex then enters the nucleus where it plays a role in transcriptional regulation. The TGF superfamily of ligands includes two major branches, characterized by TGF-β/activin/nodal and Bone Morphogenetic Proteins (BMPs).

Signals mediated by bone morphogenetic protein (BMP) ligands serve diverse roles throughout the life of vertebrates. During embryogenesis, the dorsoventral axis is established by BMP signaling gradients formed by the coordinated expression of ligands, receptors, co-receptors, and soluble antagonists. Excess BMP signaling causes ventralization, an expansion of ventral at the expense of dorsal structures, while diminished BMP signaling causes dorsalization, an expansion of dorsal at the expense of ventral structures. BMPs are key regulators of gastrulation, mesoderm induction, organogenesis, and endochondral bone formation, and regulate the fates of multipotent cell populations. BMP signals also play critical roles in physiology and disease, and are implicated, for example, in primary pulmonary hypertension, hereditary hemorrhagic telangiectasia syndrome, fibrodysplasia ossificans progressiva, and juvenile polyposis syndrome among others.

The BMP signaling family is a diverse subset of the TGF-β superfamily. Over twenty known BMP ligands are recognized by three distinct type II (BMPRII, ActRIIa, and ActRIIb) and at least three type I (ALK2, ALK3, and ALK6) receptors. Dimeric ligands facilitate assembly of receptor heteromers, allowing the constitutively-active type II receptor serine/threonine kinases to phosphorylate type I receptor serine/threonine kinases. Activated type I receptors phosphorylate BMP-responsive (BR-) SMAD effectors (SMADs 1, 5, and 8) to facilitate nuclear translocation in complex with SMAD4, a co-SMAD that also facilitates TGF signaling. In addition, BMP signals can activate intracellular effectors such as MAPK p38 in a SMAD-independent manner. Soluble BMP antagonists such as noggin, chordin, gremlin, and follistatin limit BMP signaling by ligand sequestration.

A role for BMP signals in regulating expression of hepcidin, a peptide hormone and central regulator of systemic iron balance, has also been suggested. Hepcidin binds and promotes degradation of ferroportin, the sole iron exporter in vertebrates. Loss of ferroportin activity prevents mobilization of iron to the bloodstream from intracellular stores in enterocytes, macrophages, and hepatocytes. The link between BMP signaling and iron metabolism represents a potential target for therapeutics.

Given the tremendous structural diversity of the BMP and TGF-β superfamily at the level of ligands (>25 distinct ligands at present) and receptors (three type I and three type II receptors that recognize BMPs), and the heterotetrameric manner of receptor binding, traditional approaches for inhibiting BMP signals via soluble receptors, endogenous inhibitors, or neutralizing antibodies are not practical or effective. Endogenous inhibitors such as noggin and follistatin have limited specificity for ligand subclasses. Single receptors have limited affinity for ligand, whereas ligand heterotetramers exhibit rather precise specificity for particular ligands. Neutralizing antibodies are specific for particular ligands or receptors and are also limited by the structural diversity of this signaling system.

Thus, there is a continuing need for pharmacologic agents that antagonize BMP signaling pathways and that can be used to manipulate these pathways in therapeutic or experimental applications.

### SUMMARY

In one aspect, the invention relates to methods of treating or preventing a disease or condition comprising administering to a subject a compound or a pharmaceutically acceptable salt thereof having the structure represented by Formula I:

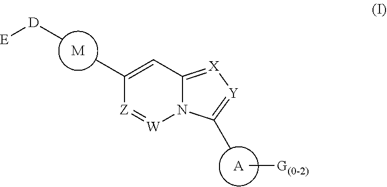

wherein A, D, E, M, G, W, X, Y, and Z are defined herein.

In some embodiments, the disease is a cancer such as colorectal cancer, sporadic colorectal cancer, acute myeloid leukemia, chronic myelogenous leukemia, non-small cell lung cancer (NSCLC), pancreatic cancer, ovarian cancer, serous ovarian cancer, epithelial ovarian cancer, melanoma, or head and neck squamous cell carcinoma (HNSCC). In other embodiments, the disease is a cancer of the central nervous system such as a glioma, astrocytic glioma, diffuse intrinsic pontine glioma (DIPG), high grade glioma (HGG), germ cell tumor, glioblastoma multiform (GBM), oligodendroglioma, pituitary tumor, or ependymoma.

In yet other embodiments, the disease is anemia, iron-refractory iron-deficient anemia (IRIDA), heterotopic ossification, nonhereditary myositis ossificans, myositis ossificans traumatica, or myositis ossificans circumscripta.

In certain embodiments, the compound is administered in a pharmaceutical composition with a pharmaceutically acceptable carrier.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which need to be independently confirmed.

### A. Definitions

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, —OCF₃, ethoxy, propoxy, tert-butoxy and the like.

The term “cycloalkyloxy” refers to a cycloakyl group having an oxygen attached thereto. The term “alkoxyalkyl” refers to an alkyl group substituted with an alkoxy group and may be represented by the general formula alkyl-O-alkyl.

The term “alkylaminoalkyl” refers to an alkyl group substituted with an alkylamino group.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkyl S—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

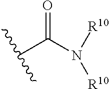

wherein each R¹⁰ independently represent a hydrogen or hydrocarbyl group, or two R¹⁰ are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

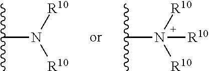

wherein each R¹⁰ independently represents a hydrogen or a hydrocarbyl group, or two R¹⁰ are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

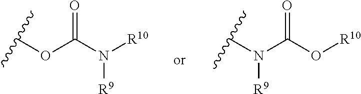

wherein R⁹ and R¹⁰ independently represent hydrogen or a hydrocarbyl group, such as an alkyl group, or R⁹ and R¹⁰ taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be susbstituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “carbonate” is art-recognized and refers to a group —OCO₂—R¹⁰, wherein R¹⁰ represents a hydrocarbyl group.

The term “carboxy”, as used herein, refers to a group represented by the formula —CO₂H.

The term “ester”, as used herein, refers to a group —C(O)OR¹⁰ wherein R¹⁰ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The term “heteroalkylamino”, as used herein, refers to an amino group substituted with a heteralkyl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyridone, benzimidazole, quinoline, isoquinoline, quinoxaline, quinazoline, indole, isoindole, indazole, benzoxazole, pyrazine, pyridazine, purine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like. Heterocyclyl groups can also be substituted by oxo groups. For example, “heterocyclyl” encompasses both pyrrolidine and pyrrolidinone.

The term “heterocycloalkyl”, as used herein, refers to an alkyl group substituted with a heterocycle group.

The term “heterocycloalkylamino”, as used herein refers to an amino group substituted with a heterocycloalkyl group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

As used herein, the term “oxo” refers to a carbonyl group. When an oxo substituent occurs on an otherwise saturated group, such as with an oxo-substituted cycloalkyl group (e.g., 3-oxo-cyclobutyl), the substituted group is still intended to be a saturated group. When a group is referred to as being substituted by an “oxo” group, this can mean that a carbonyl moiety (i.e., —C(═O)—) replaces a methylene unit (i.e., —CH₂—).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

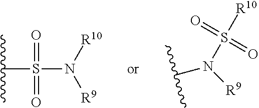

wherein R⁹ and R¹⁰ independently represents hydrogen or hydrocarbyl, such as alkyl, or R⁹ and R¹⁰ taken together with the intervening atom(s) complete a heterocycle having from 4 to 8 atoms in the ring structure.

The term “sulfoxide” is art-recognized and refers to the group —S(O)—R¹⁰, wherein R¹⁰ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R¹⁰, wherein R¹⁰ represents a hydrocarbyl.

The term “thioalkyl”, as used herein, refers to an alkyl group substituted with a thiol group.

The term “thioester”, as used herein, refers to a group —C(O)SR¹⁰ or —SC(O)R¹⁰ wherein R¹⁰ represents a hydrocarbyl.

The term “thioether”, as used herein, is equivalent to an ether, wherein the oxygen is replaced with a sulfur.

The term “urea” is art-recognized and may be represented by the general formula

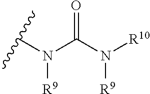

wherein R⁹ and R¹⁰ independently represent hydrogen or a hydrocarbyl, such as alkyl, or either occurrence of R⁹ taken together with R¹⁰ and the intervening atom(s) complete a heterocycle having from 4 to 8 atoms in the ring structure.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethyl silyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxylprotecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “prodrug” is intended to encompass compounds which, under physiologic conditions, are converted into the therapeutically active agents of the present invention (e.g., a compound of formula I). A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present invention. In certain embodiments, some or all of the compounds of formula I in a formulation represented above can be replaced with the corresponding suitable prodrug, e.g., wherein a hydroxyl in the parent compound is presented as an ester or a carbonate or carboxylic acid present in the parent compound is presented as an ester.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Of course, when a variable is present in more than one instance, it may be the same or different in each occurrence. In other words, each variable is independent from the other. In some aspects, a structure of a compound can be represented by a formula:

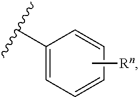

which is understood to be equivalent to a formula:

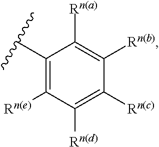

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance. Likewise, when a group R is defined as four substituents, R is understood to represent four independent substituents, R^{a}, R^{b}, R^{c}, and R^{d}.

Unless indicated to the contrary, the substituents are not limited to any particular order or arrangement.

The following abbreviations are used herein. DMF: dimethyl formamide. EtOAc: ethyl acetate. THF: tetrahydrofuran. DIPEA or DIEA: diisopropylethylamine. HOBt: 1-hydroxybenzotriazole. EDC: 1-ethyl-3-[3-dimethylaminopropyl]carbodiimide hydrochloride. DMSO: dimethylsulfoxide. DMAP: 4-Dimethylaminopyridine. RT: Room temperature. h: Hours. Min: Minutes. DCM: Dichloromethane. MeCN: Acetonitrile. MeOH: methanol. iPrOH: 2-Propanol. n-BuOH: 1-Butanol.

### B. Compounds

In one aspect, the invention relates to compounds, or pharmaceutically acceptable salts thereof, useful as BMP inhibitors. For example, the compounds of Formula I, II, or III may be used to treat or prevent a disease or condition. In general, it is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

In certain embodiments of the methods disclosed herein, the compound has the structure represented by Formula I:

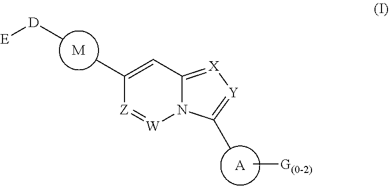

wherein:
W, X, Y, and Z are independently N or CH;
A is optionally substituted cycloalkyl, heterocyclyl, aryl or heteroaryl; G is selected from CF₃, halogen, CN, alkyl, aryl, heteroaryl, NR₁R₂, CHR³R⁴, S(O)NR¹R², S(O)₂NR¹R², SOR¹, or SO₂R¹;
M is optionally substituted aryl or heteroaryl;
D is selected from a bond, O, CR³R⁴, NR¹R², SR¹, SOR¹, or SO₂R¹;
E is absent or selected from H, CF₃, halogen, CN, alkyl, aryl, heteroaryl, C₃-C₁₂ cycloalkyl or C₃-C₁₂ heterocyclyl or C₃-C₁₂ cycloalkylalkyl or C₃-C₁₂ heterocyclylalkyl;
R¹ is absent or selected from H, alkyl, aryl, or heteroaryl;
R² is selected from H, alkyl, aryl, heteroaryl, or COR¹, or
   R¹ and R² form a C₃-C₁₂ cycloalkyl or C₃-C₁₂ heterocyclyl containing O, N and/or S;
R³ is selected from H, alkyl, aryl, or heteroaryl; and
R⁴ is selected from H, alkyl, aryl, heteroaryl, or COR¹, or
R³ and R⁴ form a C₃-C₁₂ cycloalkyl or a C₃-C₁₂ heterocyclyl containing O, N and/or S; or a pharmaceutically acceptable salt thereof.

In some embodiments, W is CH. In some embodiments, Z is CH. In some embodiments, Z is N. In some embodiments, X is N. In some embodiments, Y is N.

In some embodiments,

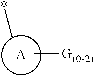

is:

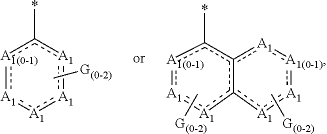

wherein A₁ is independently O, CR³R⁴, NH, or NR₁, or can join with another A₁ to form C₃-C₁₂ cycloalkyl, C₃-C₁₂ cycloalkenyl, aryl, heteroaryl, or C₃-C₁₂ heterocyclyl.

In some embodiments, A is chosen from the following:

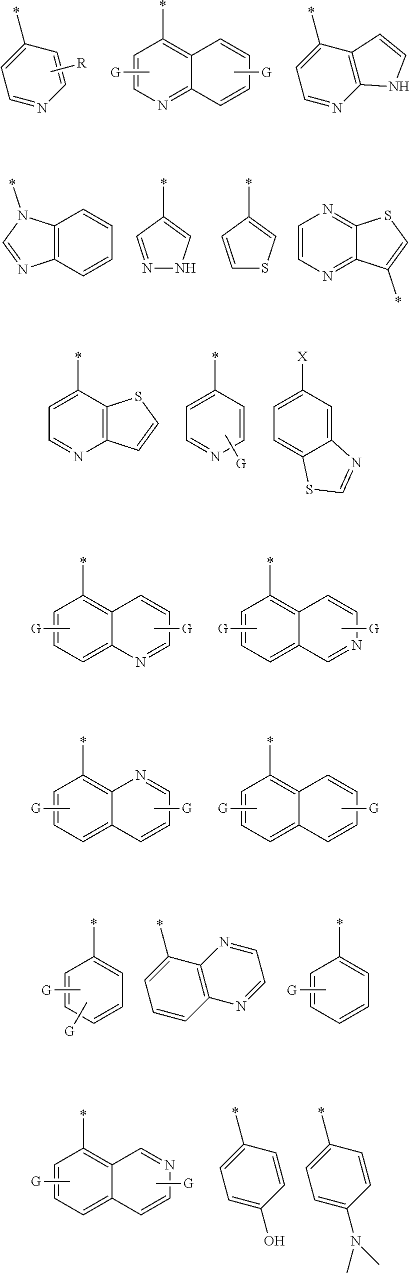

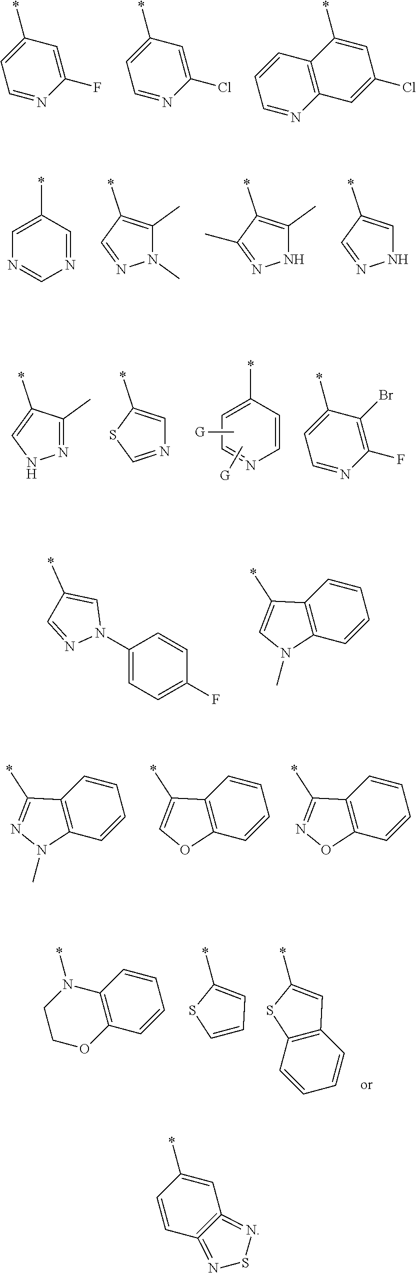

In some embodiments, M is optionally substituted with one or more G, and is selected from aryl or heteroaryl. For example, in some embodiments, M is optionally substituted phenyl or pyridine.

Also disclosed are compounds of formula I, where M, D, and E together form:

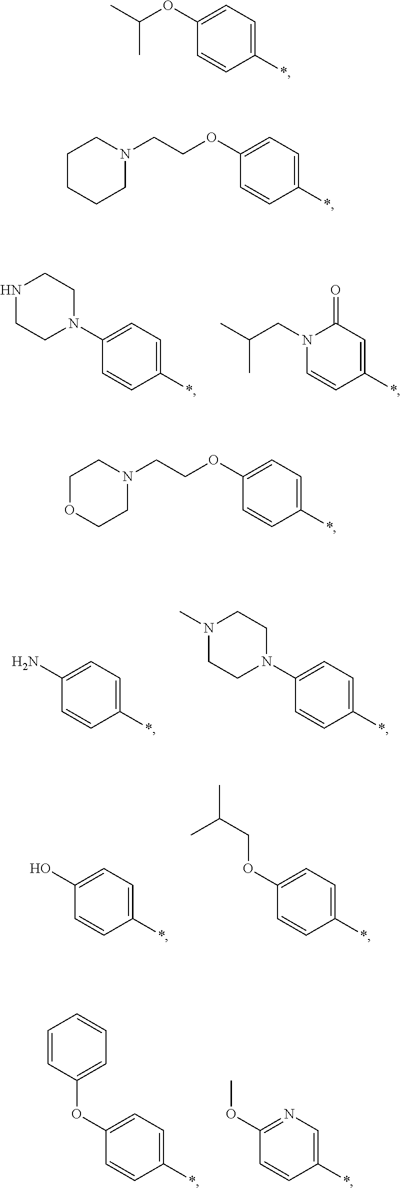

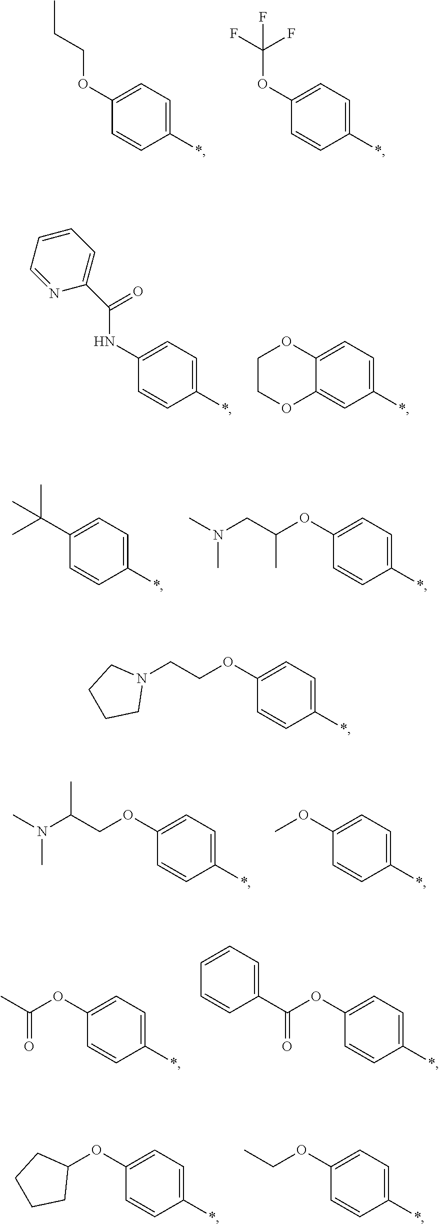

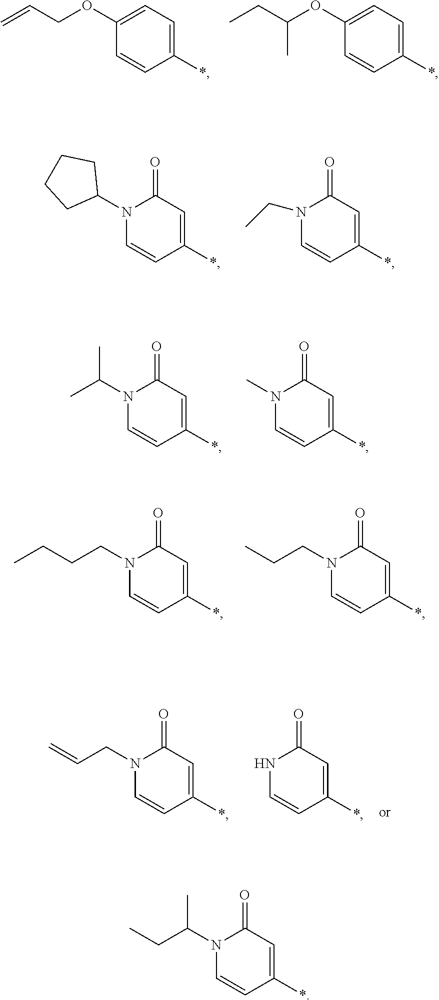

Also disclosed are compounds of formula (I), having a structure selected from:

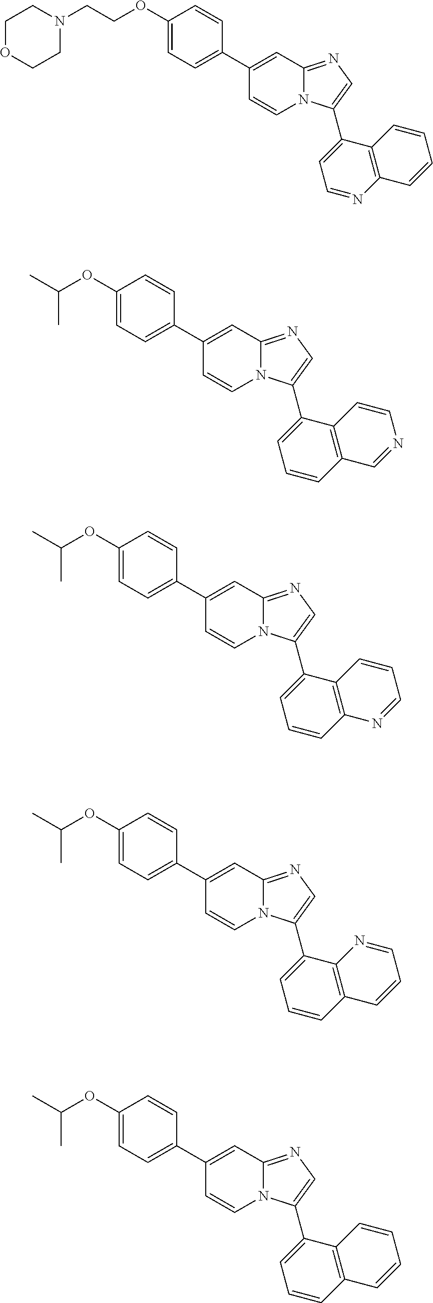 [SMILES: c1ccc2c(-c3cnc4cc(-c5ccc(OCCN6CCOCC6)cc5)ccn34)ccnc2c1 ; CC(C)Oc1ccc(-c2ccn3c(-c4cccc5cnccc45)cnc3c2)cc1 ; CC(C)Oc1ccc(-c2ccn3c(-c4cccc5ncccc45)cnc3c2)cc1 ; CC(C)Oc1ccc(-c2ccn3c(-c4cccc5cccnc45)cnc3c2)cc1 ; CC(C)Oc1ccc(-c2ccn3c(-c4cccc5ccccc45)cnc3c2)cc1]

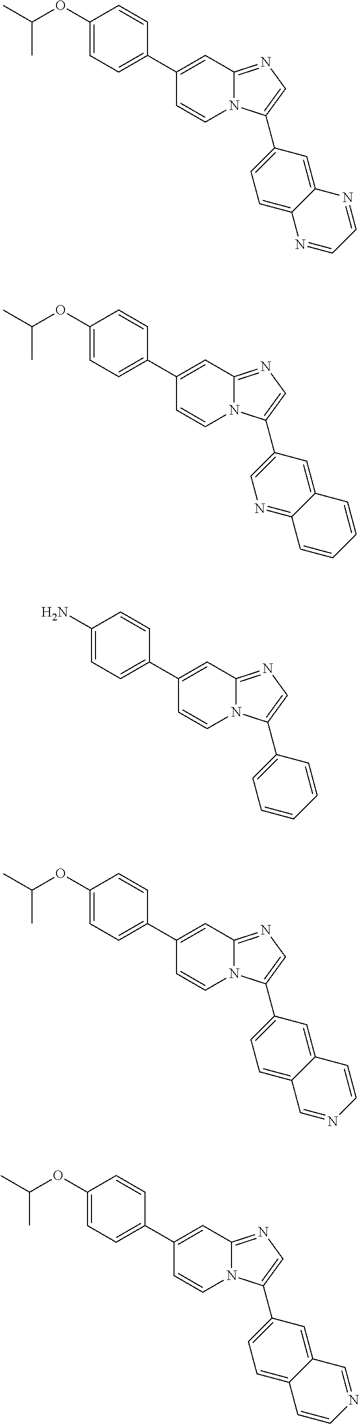 [SMILES: CC(C)Oc1ccc(-c2ccn3c(-c4ccc5nccnc5c4)cnc3c2)cc1 ; CC(C)Oc1ccc(-c2ccn3c(-c4cnc5ccccc5c4)cnc3c2)cc1 ; Nc1ccc(-c2ccn3c(-c4ccccc4)cnc3c2)cc1 ; CC(C)Oc1ccc(-c2ccn3c(-c4ccc5cnccc5c4)cnc3c2)cc1 ; CC(C)Oc1ccc(-c2ccn3c(-c4ccc5ccncc5c4)cnc3c2)cc1]

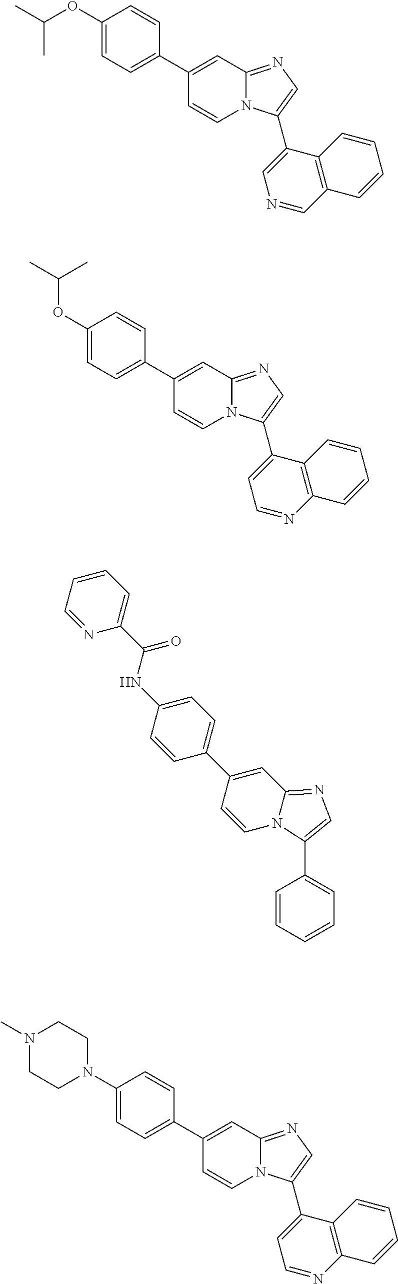 [SMILES: CC(C)Oc1ccc(-c2ccn3c(-c4cncc5ccccc45)cnc3c2)cc1 ; CC(C)Oc1ccc(-c2ccn3c(-c4ccnc5ccccc45)cnc3c2)cc1 ; O=C(Nc1ccc(-c2ccn3c(-c4ccccc4)cnc3c2)cc1)c1ccccn1 ; CN1CCN(c2ccc(-c3ccn4c(-c5ccnc6ccccc56)cnc4c3)cc2)CC1]

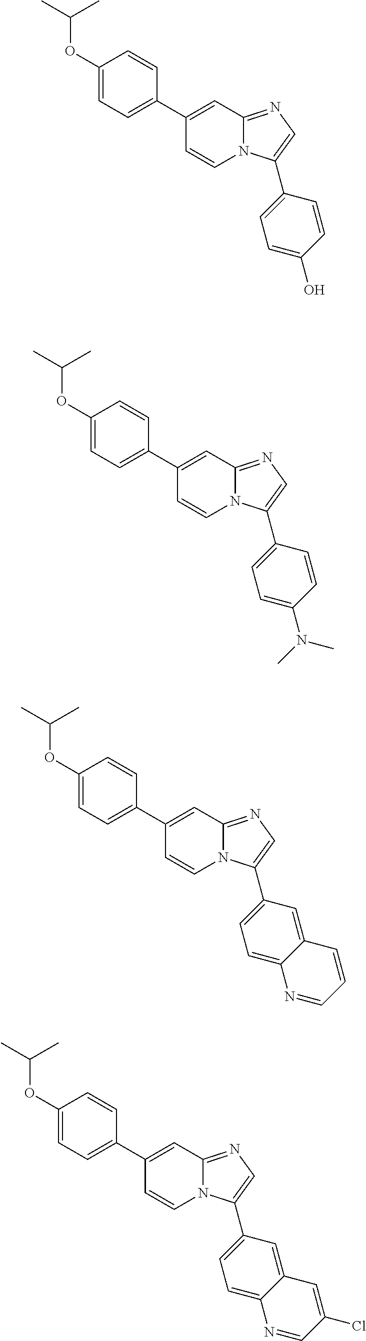

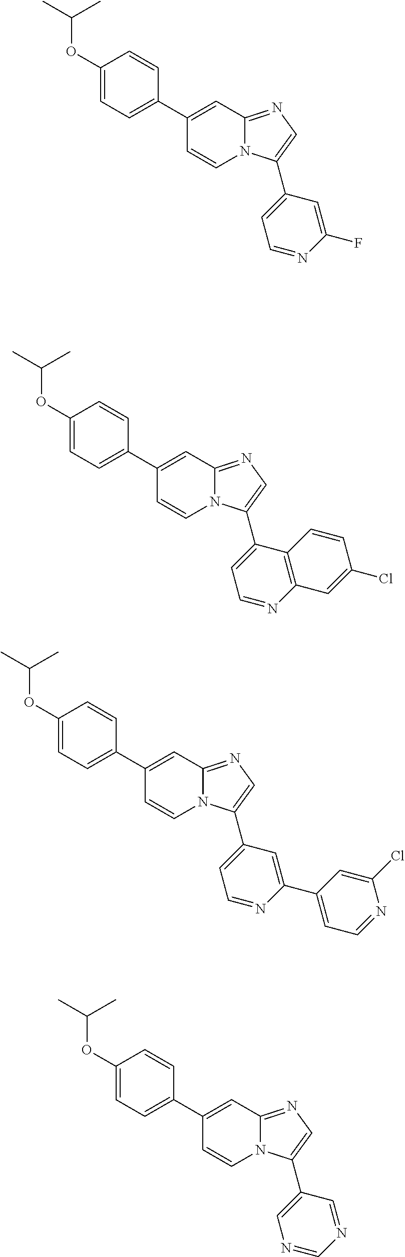 [SMILES: CC(C)Oc1ccc(-c2ccn3c(-c4ccnc(F)c4)cnc3c2)cc1 ; CC(C)Oc1ccc(-c2ccn3c(-c4ccnc5cc(Cl)ccc45)cnc3c2)cc1 ; CC(C)Oc1ccc(-c2ccn3c(-c4ccnc(-c5ccnc(Cl)c5)c4)cnc3c2)cc1 ; CC(C)Oc1ccc(-c2ccn3c(-c4cncnc4)cnc3c2)cc1]

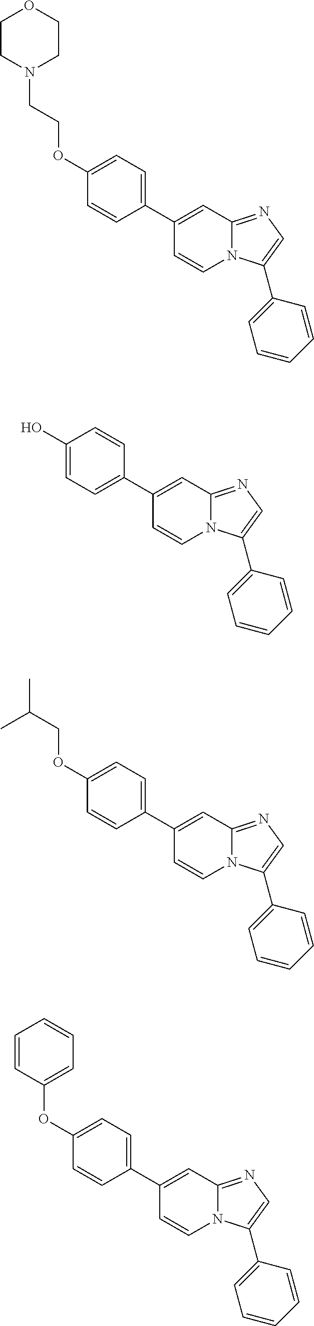 [SMILES: c1ccc(-c2cnc3cc(-c4ccc(OCCN5CCOCC5)cc4)ccn23)cc1 ; Oc1ccc(-c2ccn3c(-c4ccccc4)cnc3c2)cc1 ; CC(C)COc1ccc(-c2ccn3c(-c4ccccc4)cnc3c2)cc1 ; c1ccc(Oc2ccc(-c3ccn4c(-c5ccccc5)cnc4c3)cc2)cc1]

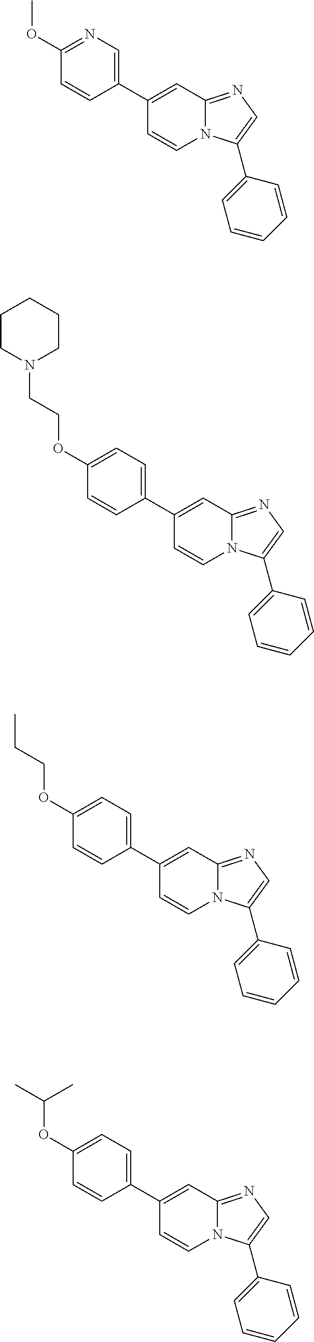 [SMILES: COc1ccc(-c2ccn3c(-c4ccccc4)cnc3c2)cn1 ; c1ccc(-c2cnc3cc(-c4ccc(OCCN5CCCCC5)cc4)ccn23)cc1 ; CCCOc1ccc(-c2ccn3c(-c4ccccc4)cnc3c2)cc1 ; CC(C)Oc1ccc(-c2ccn3c(-c4ccccc4)cnc3c2)cc1]

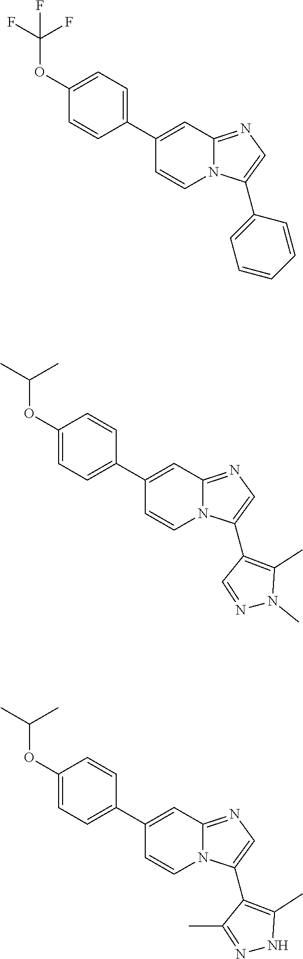

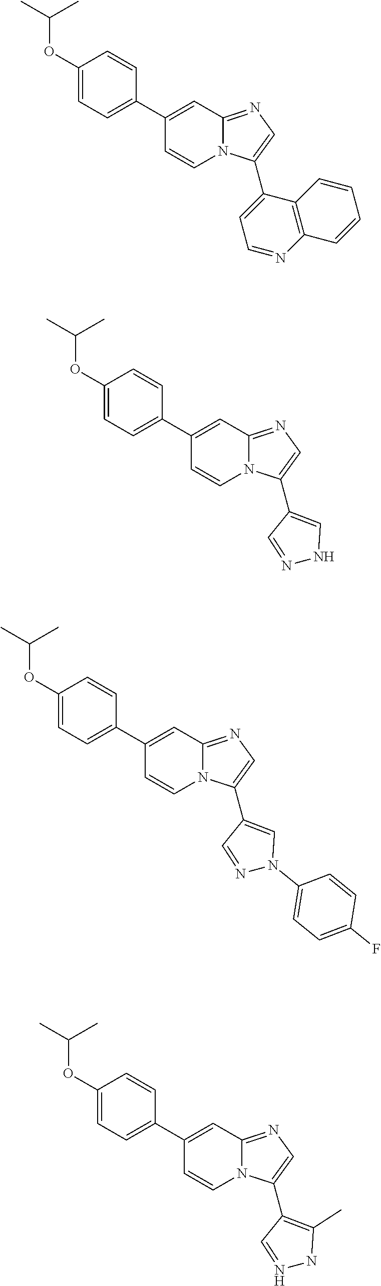 [SMILES: CC(C)Oc1ccc(-c2ccn3c(-c4ccnc5ccccc45)cnc3c2)cc1 ; CC(C)Oc1ccc(-c2ccn3c(-c4cn[nH]c4)cnc3c2)cc1 ; CC(C)Oc1ccc(-c2ccn3c(-c4cnn(-c5ccc(F)cc5)c4)cnc3c2)cc1 ; Cc1[nH]ncc1-c1cnc2cc(-c3ccc(OC(C)C)cc3)ccn12]

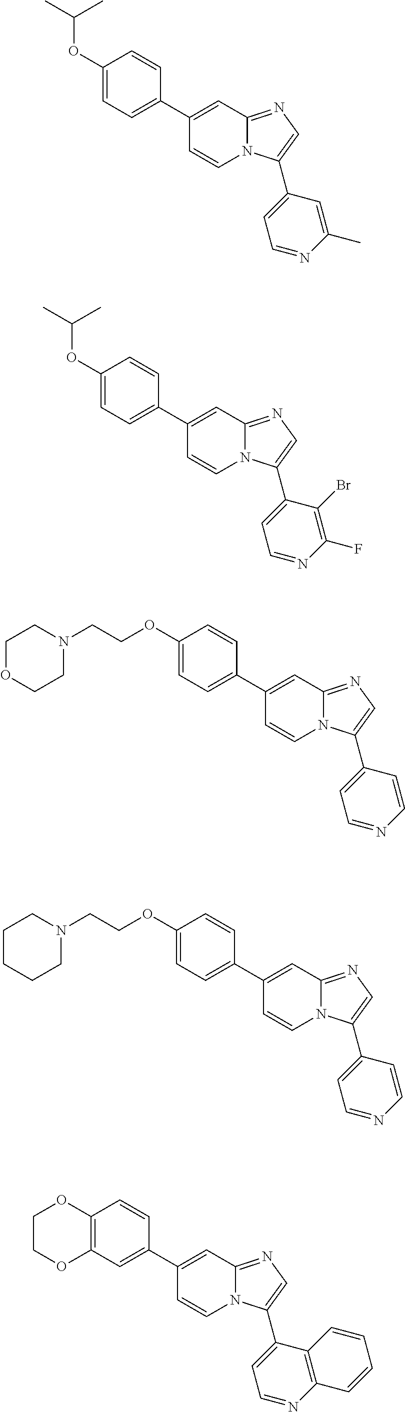

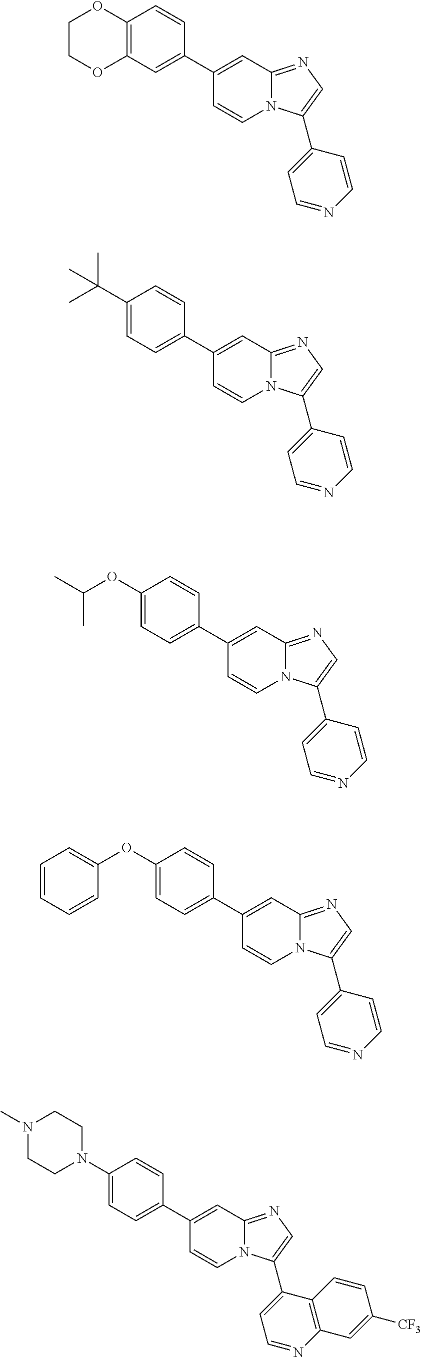 [SMILES: c1cc(-c2cnc3cc(-c4ccc5c(c4)OCCO5)ccn23)ccn1 ; CC(C)(C)c1ccc(-c2ccn3c(-c4ccncc4)cnc3c2)cc1 ; CC(C)Oc1ccc(-c2ccn3c(-c4ccncc4)cnc3c2)cc1 ; c1ccc(Oc2ccc(-c3ccn4c(-c5ccncc5)cnc4c3)cc2)cc1 ; CN1CCN(c2ccc(-c3ccn4c(-c5ccnc6cc(C(F)(F)F)ccc56)cnc4c3)cc2)CC1]

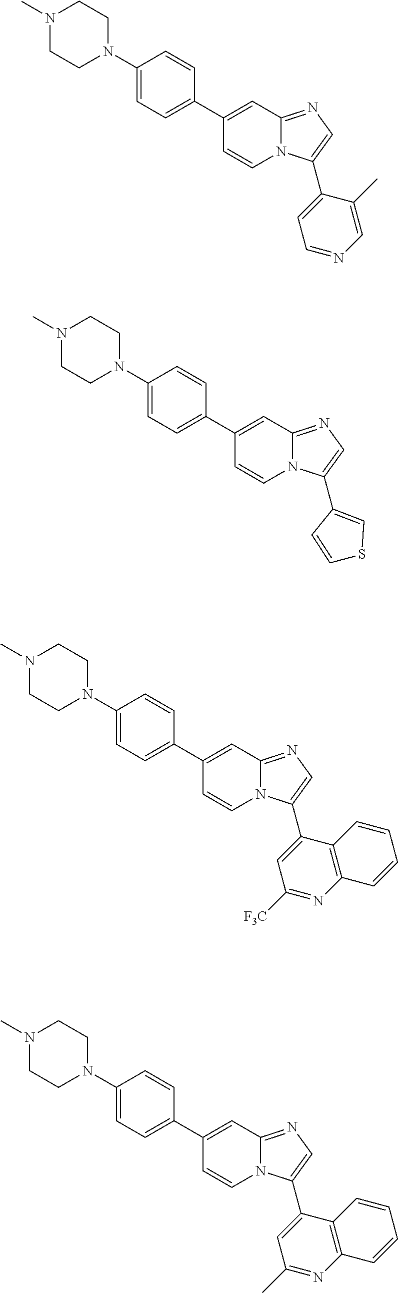

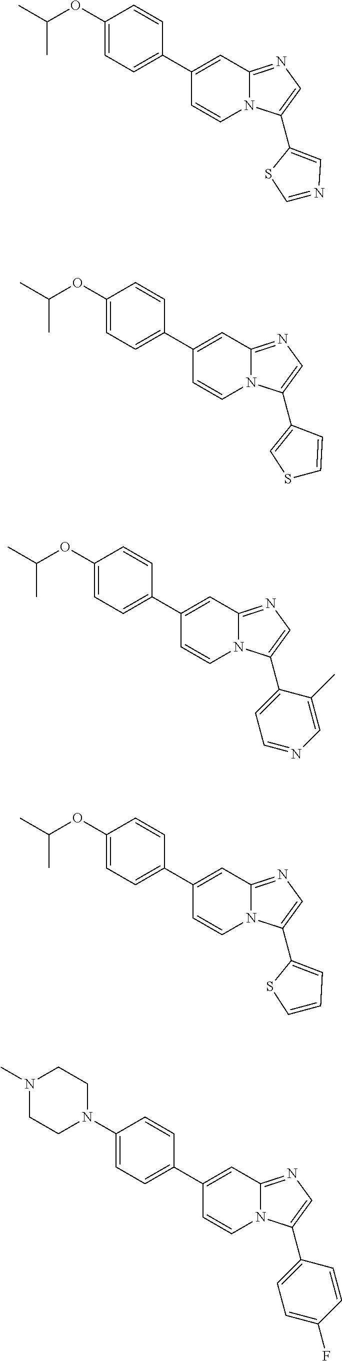

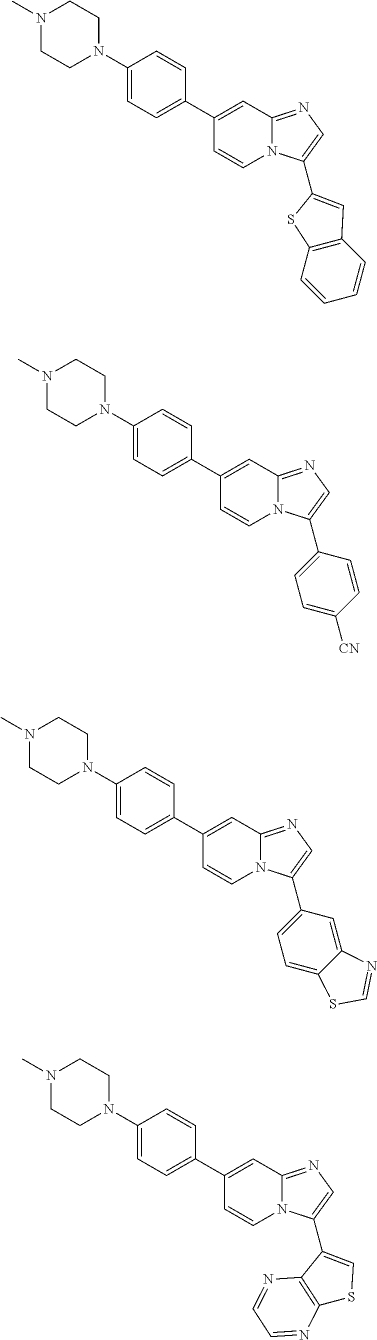 [SMILES: CN1CCN(c2ccc(-c3ccn4c(-c5cc6ccccc6s5)cnc4c3)cc2)CC1 ; CN1CCN(c2ccc(-c3ccn4c(-c5ccc(C#N)cc5)cnc4c3)cc2)CC1 ; CN1CCN(c2ccc(-c3ccn4c(-c5ccc6scnc6c5)cnc4c3)cc2)CC1 ; CN1CCN(c2ccc(-c3ccn4c(-c5csc6nccnc56)cnc4c3)cc2)CC1]

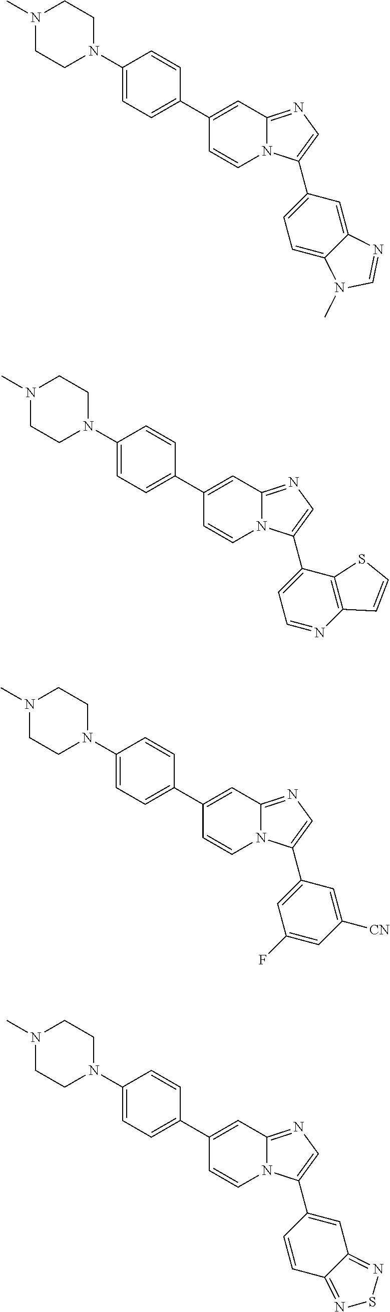 [SMILES: CN1CCN(c2ccc(-c3ccn4c(-c5ccc6c(c5)ncn6C)cnc4c3)cc2)CC1 ; CN1CCN(c2ccc(-c3ccn4c(-c5ccnc6ccsc56)cnc4c3)cc2)CC1 ; CN1CCN(c2ccc(-c3ccn4c(-c5cc(F)cc(C#N)c5)cnc4c3)cc2)CC1 ; CN1CCN(c2ccc(-c3ccn4c(-c5ccc6nsnc6c5)cnc4c3)cc2)CC1]

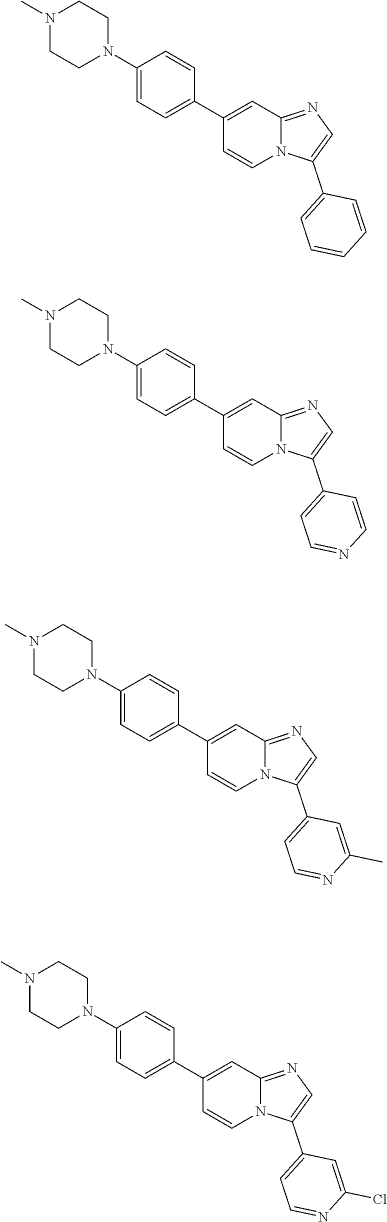

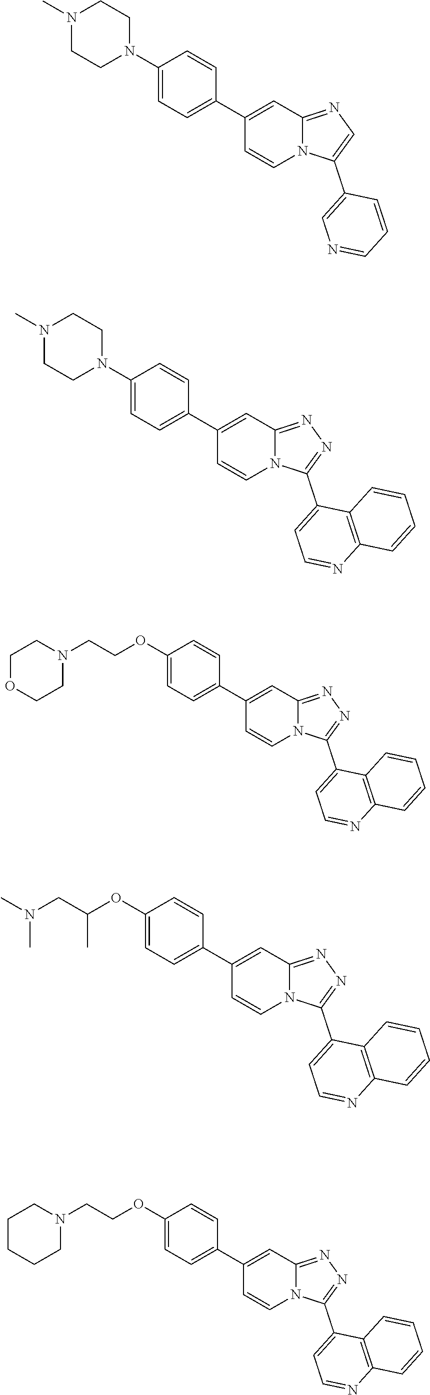 [SMILES: CN1CCN(c2ccc(-c3ccn4c(-c5cccnc5)cnc4c3)cc2)CC1 ; CN1CCN(c2ccc(-c3ccn4c(-c5ccnc6ccccc56)nnc4c3)cc2)CC1 ; c1ccc2c(-c3nnc4cc(-c5ccc(OCCN6CCOCC6)cc5)ccn34)ccnc2c1 ; CC(CN(C)C)Oc1ccc(-c2ccn3c(-c4ccnc5ccccc45)nnc3c2)cc1 ; c1ccc2c(-c3nnc4cc(-c5ccc(OCCN6CCCCC6)cc5)ccn34)ccnc2c1]

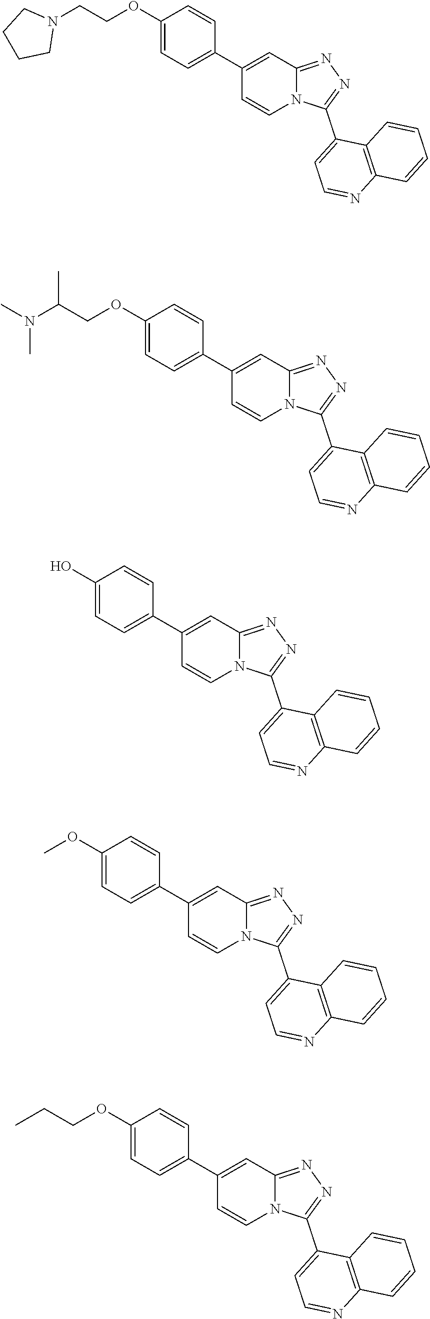 [SMILES: c1ccc2c(-c3nnc4cc(-c5ccc(OCCN6CCCC6)cc5)ccn34)ccnc2c1 ; CC(COc1ccc(-c2ccn3c(-c4ccnc5ccccc45)nnc3c2)cc1)N(C)C ; Oc1ccc(-c2ccn3c(-c4ccnc5ccccc45)nnc3c2)cc1 ; COc1ccc(-c2ccn3c(-c4ccnc5ccccc45)nnc3c2)cc1 ; CCCOc1ccc(-c2ccn3c(-c4ccnc5ccccc45)nnc3c2)cc1]

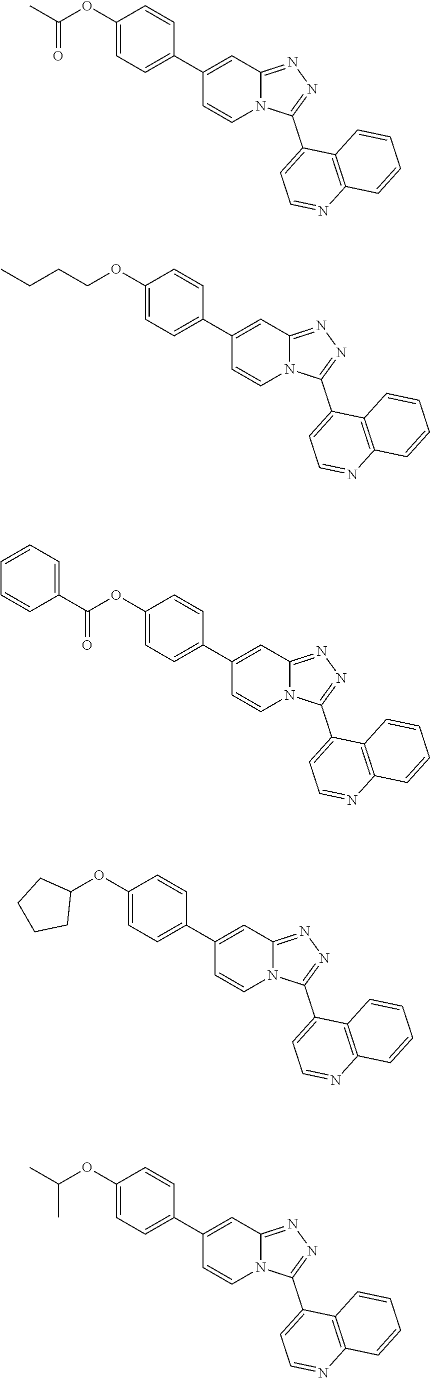 [SMILES: CC(=O)Oc1ccc(-c2ccn3c(-c4ccnc5ccccc45)nnc3c2)cc1 ; CCCCOc1ccc(-c2ccn3c(-c4ccnc5ccccc45)nnc3c2)cc1 ; O=C(Oc1ccc(-c2ccn3c(-c4ccnc5ccccc45)nnc3c2)cc1)c1ccccc1 ; c1ccc2c(-c3nnc4cc(-c5ccc(OC6CCCC6)cc5)ccn34)ccnc2c1 ; CC(C)Oc1ccc(-c2ccn3c(-c4ccnc5ccccc45)nnc3c2)cc1]

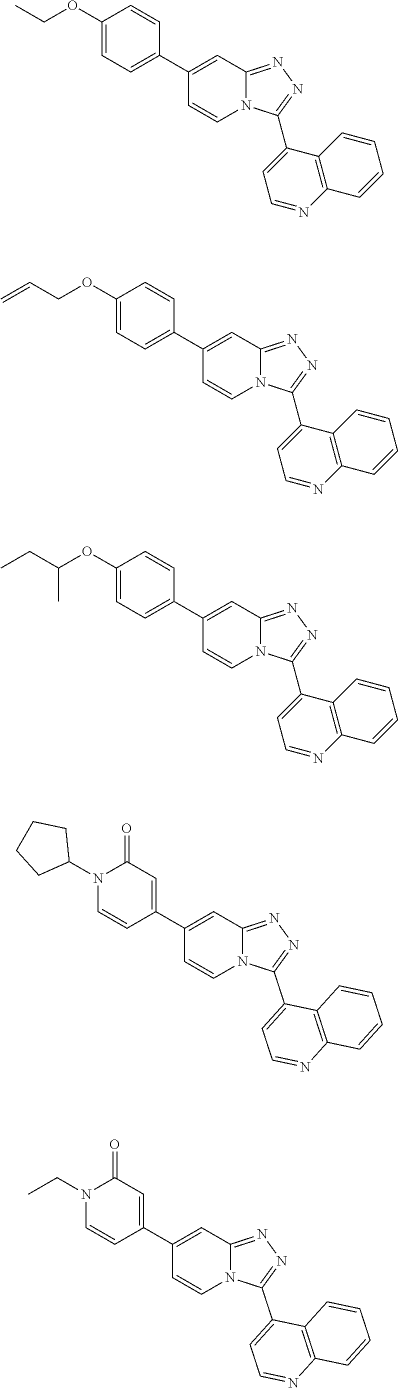 [SMILES: CCOc1ccc(-c2ccn3c(-c4ccnc5ccccc45)nnc3c2)cc1 ; C=CCOc1ccc(-c2ccn3c(-c4ccnc5ccccc45)nnc3c2)cc1 ; CCC(C)Oc1ccc(-c2ccn3c(-c4ccnc5ccccc45)nnc3c2)cc1 ; O=c1cc(-c2ccn3c(-c4ccnc5ccccc45)nnc3c2)ccn1C1CCCC1 ; CCn1ccc(-c2ccn3c(-c4ccnc5ccccc45)nnc3c2)cc1=O]

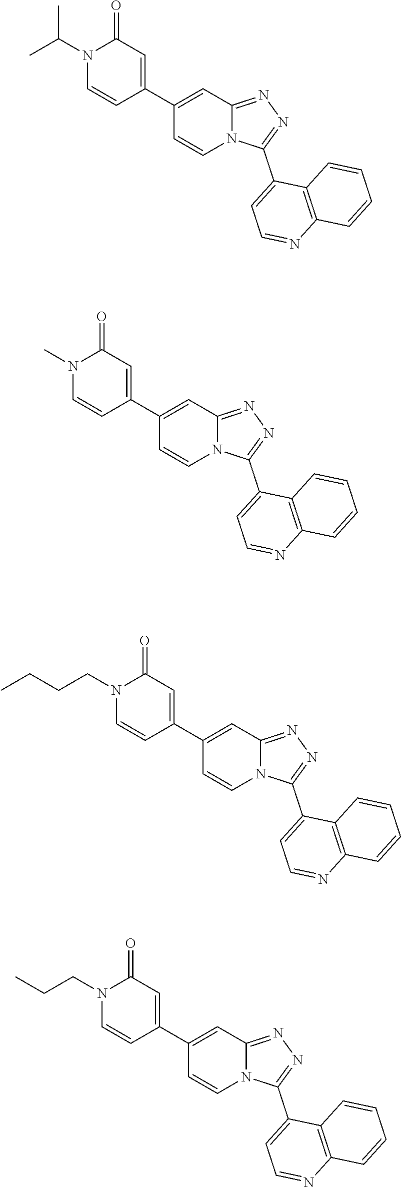 [SMILES: CC(C)n1ccc(-c2ccn3c(-c4ccnc5ccccc45)nnc3c2)cc1=O ; Cn1ccc(-c2ccn3c(-c4ccnc5ccccc45)nnc3c2)cc1=O ; CCCCn1ccc(-c2ccn3c(-c4ccnc5ccccc45)nnc3c2)cc1=O ; CCCn1ccc(-c2ccn3c(-c4ccnc5ccccc45)nnc3c2)cc1=O]

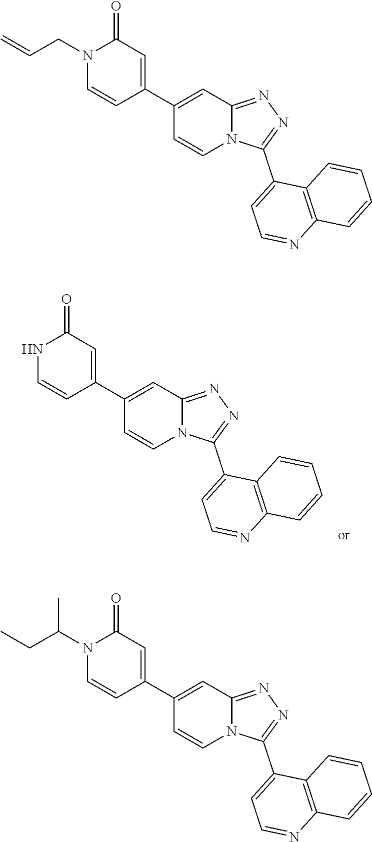 [SMILES: C=CCn1ccc(-c2ccn3c(-c4ccnc5ccccc45)nnc3c2)cc1=O ; O=c1cc(-c2ccn3c(-c4ccnc5ccccc45)nnc3c2)cc[nH]1 ; CCC(C)n1ccc(-c2ccn3c(-c4ccnc5ccccc45)nnc3c2)cc1=O]

or a pharmaceutically acceptable salt thereof.

In certain embodiments of the methods disclosed herein, the compound has the structure represented by Formula II:

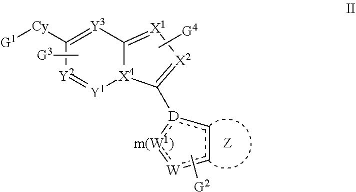

wherein:

X¹ is N or CR⁵;

X² and X⁴ are independently N or CR⁵;

Y¹, Y², and Y³ are independently N or CR⁵;

D is C or N;

W is N or O;

W¹ is N, O or C;

Cy is substituted with one or more G¹, and is selected from C₃-C₁₂ cycloalkyl, C₃-C₁₂ cycloalkenyl, aryl, heteroaryl, or C₃-C₁₂ heterocyclyl

G¹-G⁵ are absent or independently selected from H, halogen, CN, CF₃, C₁₋₁₀ alkyl, C₃₋₁₀ cycloalkyl, OC₁₋₁₀ alkyl optionally substituted with a C₃₋₈ membered ring containing C, O, S or N, optionally substituted with one or more R⁶, NR⁶C₁₋₁₀ alkyl optionally substituted with or a C₃₋₈ membered ring containing C, O, S or N, optionally substituted with one or more R⁶;

R⁵ and R⁶ are independently selected from H, halogen, CN, CF₃, C₁₋₁₀ alkyl, C₃₋₁₀ cycloalkyl, OC₁₋₁₀ alkyl optionally substituted with a C₃₋₈ membered ring containing C, O, S or N;

Z is optionally substituted with one or more R₅, and is selected from C₃-C₁₂ cycloalkyl, C₃-C₁₂ cycloalkenyl, aryl, heteroaryl, or C₃-C₁₂ heterocyclyl; and

m is 1 or 2.

In some embodiments, D is C and m is 2. In some embodiments, W is N.

In some embodiments,

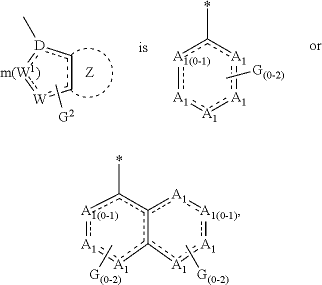

wherein A₁ is independently O, CR¹R² or NH or NR¹ or NR¹R², or can join with another A₁ to form C₃-C₁₂ cycloalkyl, C₃-C₁₂ cycloalkenyl, aryl, heteroaryl or C₃-C₁₂ heterocyclyl.

In certain embodiments of the methods disclosed herein, the compound has a structure represented by Formula III:

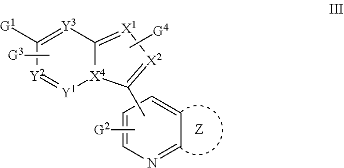

wherein:

X¹, X², and X⁴ are independently N or CR⁵;

Y¹, Y², and Y³ are independently N or CR⁵;

G¹-G⁵ are absent or independently selected from H, halogen, CN, CF₃, C₁₋₁₀ alkyl, C₃₋₁₀ cycloalkyl, OC₁₋₁₀ alkyl optionally substituted with a C₃₋₈ membered ring containing C, O, S or N, optionally substituted with one or more R⁶,

R⁵ and R⁶ are independently selected from H, halogen, CN, CF₃, C₁₋₁₀ alkyl, C₃₋₁₀ cycloalkyl, and OC₁₋₁₀ alkyl optionally substituted with a C₃₋₈ membered ring containing C, O, S or N; and

Z is optionally substituted with one or more G⁵, and is selected from C₃-C₁₂ cycloalkyl, C₃-C₁₂ cycloalkenyl, aryl, heteroaryl, or C₃-C₁₂ heterocyclyl.

In certain embodiments of the methods disclosed herein, the compound has the formula:

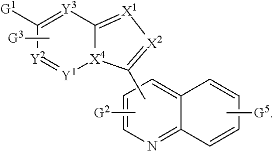

In some embodiments, i. X¹ is N, X⁴ is N, and X², X⁴, Y¹, Y², and Y³ are independently C or CR⁵;

ii. X¹ is N, X² is N, Y₁ is N, and X⁴, Y², and Y³ are independently CR⁵;

iii. X¹ is N, X² is N, Y² is N, and X⁴, Y², and Y³ are independently CR⁵; or

iv. X¹ is N, and X², X⁴, Y¹, Y², and Y³ are independently CR⁵; Y¹, Y², and Y³ are independently N or CR¹.

In some embodiments, X¹ is N, X⁴ is N, and X², X⁴, Y¹, Y², and Y³ are CH. In some embodiments, X¹, X⁴, and Y¹ are N, and X², X⁴, Y², and Y³ are CH. In some embodiments, X¹, X⁴, and Y² are N, and X², X⁴, Y¹, and Y³ are CH.

In some embodiments, G² is absent. In some embodiments, G³ is absent.

The compounds disclosed herein can include all salt forms, for example, salts of both basic groups, inter alia, amines, as well as salts of acidic groups, inter alia, carboxylic acids. The following are non-limiting examples of anions that can form salts with protonated basic groups: chloride, bromide, iodide, sulfate, bisulfate, carbonate, bicarbonate, phosphate, formate, acetate, propionate, butyrate, pyruvate, lactate, oxalate, malonate, maleate, succinate, tartrate, fumarate, citrate, and the like. The following are non-limiting examples of cations that can form salts of acidic groups: ammonium, sodium, lithium, potassium, calcium, magnesium, bismuth, lysine, and the like.

### C. Pharmaceutical Compositions

In one aspect, the invention relates to methods of treating a disease or condition by administering a compound of Formula I, II, or III in a pharmaceutical composition. That is, a pharmaceutical composition can be provided comprising a therapeutically effective amount of at least one disclosed compound or at least one product of a disclosed method and a pharmaceutically acceptable carrier.

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier.

Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans, and other mammals such as equines, cattle, swine and sheep; and poultry and pets in general.

This invention includes the use of pharmaceutically acceptable salts of compounds of the invention in the compositions and methods of the present invention. The term “pharmaceutically acceptable salt” as used herein includes salts derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2-sulfonic, and other acids. Pharmaceutically acceptable salt forms can include forms wherein the ratio of molecules comprising the salt is not 1:1. For example, the salt may comprise more than one inorganic or organic acid molecule per molecule of base, such as two hydrochloric acid molecules per molecule of compound of Formula I or Formula II. As another example, the salt may comprise less than one inorganic or organic acid molecule per molecule of base, such as two molecules of compound of Formula I or Formula II per molecule of tartaric acid.

In further embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like

In such combinations the compound of the present invention and other active agents may be administered separately or in conjunction. In addition, the administration of one element can be prior to, concurrent to, or subsequent to the administration of other agent(s).

Accordingly, the subject compounds can be used alone or in combination with other agents which are known to be beneficial in the subject indications or other drugs that affect receptors or enzymes that either increase the efficacy, safety, convenience, or reduce unwanted side effects or toxicity of the disclosed compounds. The subject compound and the other agent may be coadministered, either in concomitant therapy or in a fixed combination.

In one aspect, the compound can be employed in combination with a second compound with the known side effect of modulating BMP signaling pathways.

### D. Uses of the Compounds and Compositions

In certain aspects, provided herein are methods of treating or preventing a disease or condition comprising administering to a subject a compound having the structure represented by Formula I, II, or III.

In certain embodiments, disease is a cancer. In certain embodiments, the disease is colorectal cancer, juvenile polyposis syndrome, sporadic colorectal cancer, leukemia, acute myeloid leukemia, acute megakaryoblastic leukemia (AMKL), non-Down syndrome AMKL, Down syndrome AMKL, chronic myelogenous leukemia, lung cancer, non-small cell lung cancer (NSCLC), pancreatic cancer, ovarian cancer, serous ovarian cancer, epithelial ovarian cancer, osteosarcomas, prostate cancer, bone cancer, renal cell cancer, breast cancer, melanoma, or head and neck squamous cell carcinoma (HNSCC).

In certain embodiments, the cancer is a cancer of the central nervous system. In certain embodiments, the cancer is a glioma, astrocytic glioma, diffuse intrinsic pontine glioma (DIPG), high grade glioma (HGG), germ cell tumor, glioblastoma multiform (GBM), oligodendroglioma, pituitary tumor, or ependymoma.

In other embodiments, the disease is anemia, iron-refractory iron-deficient anemia (IRIDA), heterotopic ossification, nonhereditary myositis ossificans, myositis ossificans traumatica, or myositis ossificans circumscripta.

In certain embodiments, the compound or pharmaceutical composition is administered other than directly into the central nervous system, e.g., topically, orally, nasally, intravenously, intramuscularly, intraarterially, intracapsularly, intraorbitally, intracardiacly, intradermally, intraperitoneally, transmucosally, transdermally, anally, rectally, vaginally, transtracheally, subcutaneously, subcuticularly, intraarticularly, or subcapsularly.

### E. Manufacture of a Medicament

In one aspect, the invention relates to methods for the manufacture of a medicament for treating or preventing a disease in a subject in need thereof, comprising combining a compound of formula I, II, or II with a pharmaceutical carrier.

The invention now being generally described, it will be more readily understood by reference to the following examples which are included merely for purposes of illustration of certain aspects and embodiments of the present invention, and are not intended to limit the invention.

### F. Experimental

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Chemical Synthesis

General.

All NMR spectra were recorded on a 400 MHz AMX Bruker NMR spectrometer. ¹H chemical shifts are reported in 6 values in ppm downfield with the deuterated solvent as the internal standard. Data are reported as follows: chemical shift, multiplicity (s=singlet, d=doublet, t=triplet, q=quartet, br=broad, m=multiplet), integration, coupling constant (Hz). Low resolution mass spectra were obtained on an Agilent 1200 series 6130 mass spectrometer with electrospray ionization. High resolution mass spectra were recorded on a Waters Q-TOF API-US plus Acquity system with electrospray ionization. (see Table 1, below) Analytical thin layer chromatography was performed on EM Reagent 0.25 mm silica gel 60-F plates. Analytical HPLC was performed on an Agilent 1200 series with UV detection at 215 nm and 254 nm along with ELSD detection. LC/MS: (Phenomenex-C18, 2.1×30 mm, 1 min gradient, 7%[0.1% TFA/CH₃CN]:93%[0.1% TFA/H₂O] to 95%[0.1% TFA/CH₃CN]. Preparative purification was performed on a custom HP1100 purification system (reference 16) with collection triggered by mass detection. Solvents for extraction, washing and chromatography were HPLC grade. All reagents were purchased from Aldrich Chemical Co. and were used without purification.

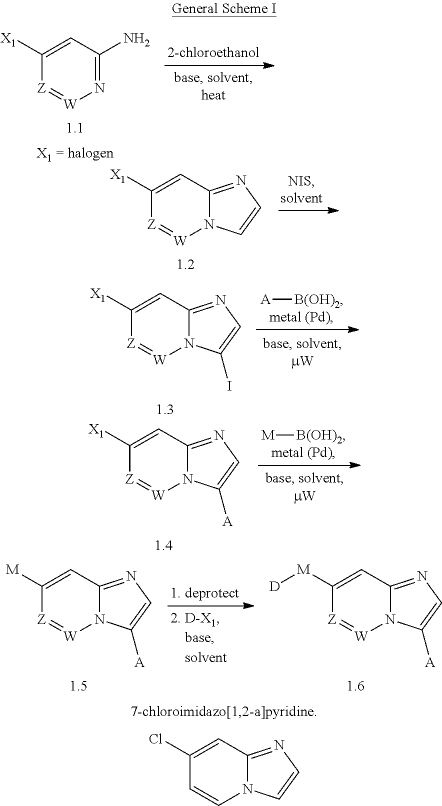

To a mixture of 4-chloropyridin-2-amine (1.0 g, 7.78 mmol, 1.0 eq) and NaHCO₃ (1.31 g, 15.56 mmol, 2.0 eq) in EtOH (18 mL) was added chloroacetaldehyde, 50% wt in water, (1.48 mL, 11.67 mmol, 1.5 eq). The reaction mixture was heated to reflux. After 10 h, the solvent was removed under reduced pressure and the residue was partitioned between EtOAc: H₂O (1:1, 100 mL). The organic layer was washed with Brine (50 mL), dried (MgSO₄), filtered and concentrated. The material was taken through without further purification.

LCMS: R_{T}=0.123 min, >98% @ 215 and 254 nM, m/z=153.0 [M+H]⁺.

### 7-chloro-3-iodoimidazo[1,2-a]pyridine

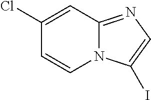 [SMILES: Clc1ccn2c(I)cnc2c1]

To a solution of 7-chloroimidazo[1,2-a]pyridine (7.78 mmol, 1.0 eq) in DMF (12 mL) at rt was added N-iodosuccinimide (1.84 g, 8.17 mmol, 1.05 eq). After 16 h, the brown slurry was diluted with H₂O (100 mL) and Brine (15 mL). The mixture was extracted with EtOAc (100 mL). The aqueous layer was re-extracted with EtOAc (100 mL) and the collected organic layers were washed with H₂O (2×20 mL), 10% sodium thiosulfate (20 mL), Brine (20 mL) and dried (MgSO₄). After filtration, the solution was concentrated. The residue was triturated with diethyl ether (15 mL) and filtered to afford an off-white solid (1.58 g, 73% yield over 2 steps). LCMS: R_{T}=0.265 min, >98% @ 215 and 254 nM, m/z=279.0 [M+H]⁺.

### 7-chloro-3-phenylimidazo[1,2-a]pyridine

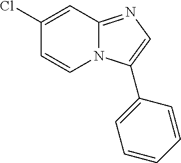 [SMILES: Clc1ccn2c(-c3ccccc3)cnc2c1]

In a μwave vial, 7-chloro-3-iodoimidazo[1,2-a]pyridine (0.39 g, 1.38 mmol, 1.0 eq), phenyl boronic acid (0.18 g, 1.45 mmol, 1.05 eq), and Pd(dppf)Cl₂ (50.5 mg, 0.07 mmol, 0.05 eq) were added. The solid mixture was evacuated under vacuo and purged with Argon (3×). To the mixture was added 1,4-dioxane (6 mL), followed by a solution of K₃PO₄ (0.59 g, 2.76 mmol, 2.0 eq) in H₂O (2.5 mL). The reaction was heated to 120° C. for 30 min under microwave irradiation. The reaction was added to EtOAc: H₂O (1:1, 120 mL). The organic layer was separated, washed with H₂O (2×25 mL), Brine (25 mL), dried (MgSO₄), filtered and concentrated. The material was purified by reverse-phase HPLC (15-40% acetonitrile: H₂O w/0.1% TFA) to provide 7-chloro-3-phenylimidazo[1,2-a]pyridine (0.30 g, 96% yield).

LCMS: R_{T}=0.458 min, >98% @ 215 and 254 nM, m/z=229.0 [M+H]⁺.

### 7-(4-isopropoxyphenyl)-3-phenylimidazo[1,2-a]pyridine

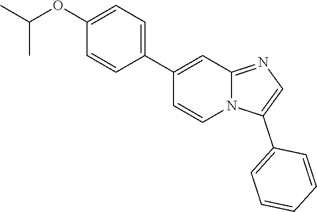 [SMILES: CC(C)Oc1ccc(-c2ccn3c(-c4ccccc4)cnc3c2)cc1]

In a μwave vial, 7-chloro-3-phenylimidazo[1,2-a]pyridine (5) (25.0 mg, 0.11 mmol, 1.0 eq), boronic acid 6 (22.0 mg, 0.121 mmol, 1.1 eq), and Pd(dppf)Cl₂ (4.0 mg, 0.006 mmol, 0.05 eq) were added. The solid mixture was evacuated under vacuo and purged with Argon (3×). To the mixture was added 1,4-dioxane (2 mL), followed by a solution of K₂CO₃ (30.0 mg, 0.22 mmol, 2.0 eq) in H₂O (1.0 mL). The reaction was heated to 150° C. for 30 min under microwave irradiation. The reaction was added to EtOAc: H₂O (1:1, 20 mL). The organic layer was separated, washed with H₂O (5 mL), Brine (5 mL), dried (MgSO₄), filtered and concentrated. The material was purified by reverse-phase HPLC (30-65% acetonitrile: H₂O w/0.1% TFA) to afford 7-(4-isopropoxyphenyl)-3-phenylimidazo[1,2-a]pyridine (5.30 mg, 15% yield).

LCMS: R_{T}=0.714 min, >98% @ 215 and 254 nM, m/z=329.0 [M+H]⁺.

### 7-chloro-3-(pyridin-4-yl)imidazo[1,2-a]pyridine

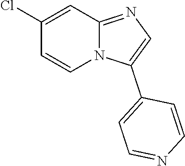 [SMILES: Clc1ccn2c(-c3ccncc3)cnc2c1]

In a μwave vial, 7-chloro-3-iodoimidazo[1,2-a]pyridine (3) (0.31 g, 1.13 mmol, 1.0 eq), 4-pyridyl boronic acid (0.15 g, 1.24 mmol, 1.1 eq), and Pd(dppf)Cl₂ (41.0 mg, 0.06 mmol, 0.05 eq) were added. The solid mixture was evacuated under vacuo and purged with Argon (3×). To the mixture was added 1,4-dioxane (5 mL), followed by a solution of K₃PO₄ (0.48 g, 2.26 mmol, 2.0 eq) in H₂O (2.0 mL). The reaction was heated to 120° C. for 30 min under microwave irradiation. The reaction was added to EtOAc: H₂O (1:1, 120 mL). The organic layer was separated, washed with H₂O (2×25 mL), Brine (25 mL), dried (MgSO₄), filtered and concentrated. The material was taken through without further purification.

LCMS: R_{T}=0.147 min, >98% @ 215 and 254 nM, m/z=230.0 [M+H]⁺.

### 4-(3-(pyridin-4-yl)imidazo[1,2-a]pyridin-7-yl)phenol

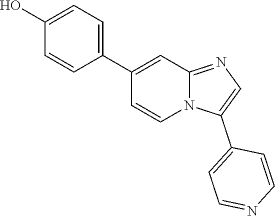 [SMILES: Oc1ccc(-c2ccn3c(-c4ccncc4)cnc3c2)cc1]

In a μwave vial, 7-chloro-3-(pyridin-4-yl)imidazo[1,2-a]pyridine (0.28 g, 1.23 mmol, 1.0 eq), 4-hydroxyphenyl boronic acid (0.19 g, 1.35 mmol, 1.1 eq), and Pd(dppf)Cl₂ (45.0 mg, 0.06 mmol, 0.05 eq) were added. The solid mixture was evacuated under vacuo and purged with Argon (3×). To the mixture was added 1,4-dioxane (5 mL), followed by a solution of K₂CO₃ (0.34 g, 2.46 mmol, 2.0 eq) in H₂O (2.0 mL). The reaction was heated to 150° C. for 30 min under microwave irradiation. The reaction was added to EtOAc: H₂O (1:1, 20 mL). The organic layer was separated, washed with H₂O (5 mL), Brine (5 mL), dried (MgSO₄), filtered and concentrated. The material was purified by reverse-phase HPLC (5-35% acetonitrile: H₂O w/0.1% TFA) to afford 4-(3-(pyridin-4-yl)imidazo[1,2-a]pyridin-7-yl)phenol (53.0 mg, 15% yield).

LCMS: R_{T}=0.343 min, >98% @ 215 and 254 nM, m/z=288.0 [M+H]⁺.

### 7-(4-(2-(piperidin-1-yl)ethoxy)phenyl)-3-(pyridin-4-yl)imidazo[1,2-a]pyridine

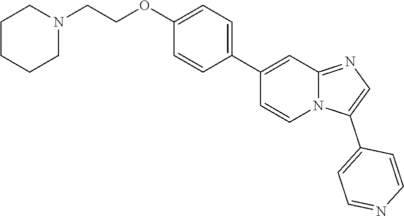 [SMILES: c1cc(-c2cnc3cc(-c4ccc(OCCN5CCCCC5)cc4)ccn23)ccn1]

To a μwave vial was added 4-(3-(pyridin-4-yl)imidazo[1,2-a]pyridin-7-yl)phenol (29.5 mg, 0.10 mmol, 1.0 eq), Cs₂CO₃ (134.0 mg, 0.411 mmol, 4.0 eq), KI (16.6 mg, 0.10 mmol, 1.0 eq), 1-(2-chloroethyl)piperidine hydrochloride (20.3 mg, 0.11 mmol, 1.1 eq) and DMF (1.5 mL). The rxn was subjected to microwave irradiatation for 10 min at 120° C. The reaction was filtered through a Celite plug and the solution was purified by reverse-phase HPLC (5-35% acetonitrile: H₂O w/0.1% TFA) to afford 7-(4-(2-(piperidin-1-yl)ethoxy)phenyl)-3-(pyridin-4-yl)imidazo[1,2-a]pyridine (15.02 mg, 38% yield).

LCMS: R_{T}=0.404 min, >98% @ 215 and 254 nM, m/z=399.0 [M+H]⁺.

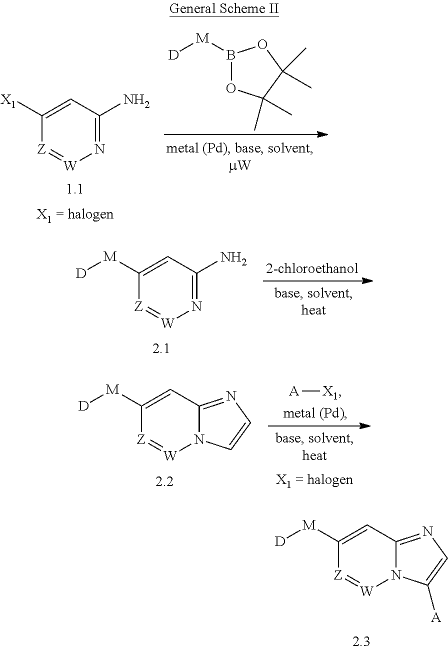

### 4-(4-(4-methylpiperazin-1-yl)phenyl)pyridin-2-amine

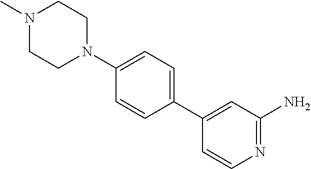 [SMILES: CN1CCN(c2ccc(-c3ccnc(N)c3)cc2)CC1]

In a μwave vial, 4-bromopyridin-2-amine (0.50 g, 2.89 mmol, 1.0 eq), boronic ester (0.92 g, 3.03 mmol, 1.05 eq), and Pd(dppf)Cl₂ (106 mg, 0.15 mmol, 0.05 eq) were added. The solid mixture was evacuated under vacuo and purged with Argon (3×). To the mixture was added 1,4-dioxane (12 mL), followed by a solution of K₃PO₄ (1.23 g, 5.78 mmol, 2.0 eq) in H₂O (5.0 mL). The reaction was heated to 120° C. for 30 min under microwave irradiation. To the reaction was added EtOAc (15 mL) and the rxn was filtered. The solid was rinsed with cold EtOAc (2 mL). The material was taken through without further purification.

LCMS: R_{T}=0.285 min, >98% @ 215 nM and ELSD, m/z=269.1 [M+H]⁺.

### 7-(4-(4-methylpiperazin-1-yl)phenyl)imidazo[1,2-a]pyridine

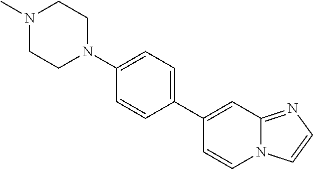 [SMILES: CN1CCN(c2ccc(-c3ccn4ccnc4c3)cc2)CC1]

To a mixture of 4-(4-(4-methylpiperazin-1-yl)phenyl)pyridin-2-amine (2.89 mmol, 1.0 eq) and NaHCO₃ (0.49 g, 5.78 mmol, 2.0 eq) in EtOH (30 mL) was added chloroacetaldehyde, 50% wt in water, (0.56 mL, 4.34 mmol, 1.5 eq). The reaction mixture was heated to reflux. After 18 h, the solvent was removed under reduced pressure and the residue was partitioned between EtOAc: H₂O (1:1, 100 mL). The organic layer was washed with Brine (50 mL), dried (MgSO₄), filtered and concentrated. The material was taken through without further purification.

LCMS: R_{T}=0.343 min, >90% @ 215 nM and ELSD, m/z=293.1 [M+H]⁺.

### 7-(7-(4-(4-methylpiperazin-1-yl)phenyl)imidazo[1,2-a]pyridin-3-yl)thieno[3,2-b]pyridine

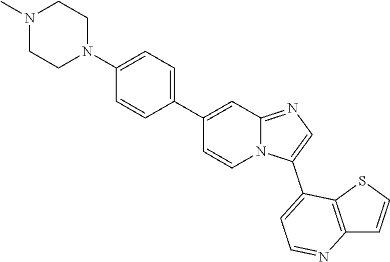 [SMILES: CN1CCN(c2ccc(-c3ccn4c(-c5ccnc6ccsc56)cnc4c3)cc2)CC1]

In a μwave vial, 7-(4-(4-methylpiperazin-1-yl)phenyl)imidazo[1,2-a]pyridine (23 mg, 0.08 mmol, 1.1 eq), 7-chlorothieno[3,2-b]pyridine (8 μL, 0.071 mmol, 1.0 eq), KOAc (14.0 mg, 0.143 mmol, 2.0 eq) and Pd(OAc)₂ (˜1 mg, 0.001 eq) were added, followed by the addition of DMA (1.5 mL). The reaction was heated to 200° C. for 30 min under microwave irradiation. To the reaction was added DMSO (0.5 mL) and after filtration through a Celite plug, the solution was purified by reverse-phase HPLC (20-55% acetonitrile: H₂O w/0.1% TFA) to afford 7-(7-(4-(4-methylpiperazin-1-yl)phenyl)imidazo[1,2-a]pyridin-3-yl)thieno[3,2-b]pyridine (14.0 mg, 47% yield).

LCMS: R_{T}=0.361 min, >98% @ 215 nM and ELSD, m/z=370.1 [M+H]⁺.

### 7-(4-isopropoxyphenyl)imidazo[1,2-a]pyridine

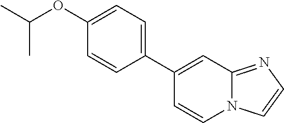 [SMILES: CC(C)Oc1ccc(-c2ccn3ccnc3c2)cc1]

Compound 7-(4-isopropoxyphenyl)imidazo[1,2-a]pyridine was prepared in a similar manner to 7-(4-(4-methylpiperazin-1-yl)phenyl)imidazo[1,2-a]pyridine.

LCMS: R_{T}=0.578 min, >98% @ 220 and 254 nM, m/z=253.1 [M+H]⁺.

### 3-iodo-7-(4-isopropoxyphenyl)imidazo[1,2-a]pyridine

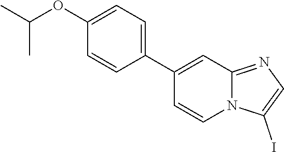 [SMILES: CC(C)Oc1ccc(-c2ccn3c(I)cnc3c2)cc1]

To a solution of compound 7-(4-isopropoxyphenyl)imidazo[1,2-a]pyridine (2.89 mmol, 1.0 eq) in DMF (20 mL) at rt was added N-iodosuccinimide (0.68 g, 3.03 mmol, 1.05 eq). After 16 h, the brown slurry was diluted with H₂O (100 mL) and Brine (15 mL). The mixture was extracted with EtOAc (100 mL). The aqueous layer was re-extracted with EtOAc (100 mL) and the collected organic layers were washed with H₂O (2×20 mL), 10% sodium thiosulfate (20 mL), Brine (20 mL) and dried (MgSO₄). After filtration, the solution was concentrated and the material was taken through without further purification.

LCMS: R_{T}=0.640 min, >95% @ 220 nM and ELSD, m/z=378.9 [M+H]⁺.

### 7-(4-isopropoxyphenyl)-3-(2-methylpyridin-4-yl)imidazo[1,2-a]pyridine

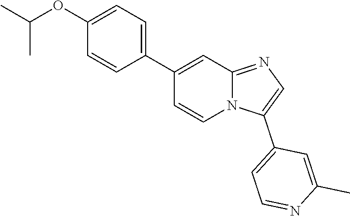 [SMILES: Cc1cc(-c2cnc3cc(-c4ccc(OC(C)C)cc4)ccn23)ccn1]

In a μwave vial, 3-iodo-7-(4-isopropoxyphenyl)imidazo[1,2-a]pyridine (35 mg, 0.093 mmol, 1.0 eq), (2-methylpyridin-4-yl)boronic acid (15 mg, 0.11 mmol, 1.2 eq), and Pd(dppf)Cl₂ (4.0 mg, 0.005 mmol, 0.05 eq) were added. The solid mixture was evacuated under vacuo and purged with Argon (3×). To the mixture was added 1,4-dioxane (2 mL), followed by a solution of K₃PO₄ (40 mg, 0.19 mmol, 2.0 eq) in H₂O (0.5 mL). The reaction was heated to 120° C. for 30 min under microwave irradiation. The reaction was added to EtOAc: H₂O (1:1, 20 mL). The organic layer was separated, washed with H₂O (2×25 mL), Brine (25 mL), dried (MgSO₄), filtered and concentrated. The residue was purified by reverse-phase HPLC (20-55% acetonitrile: H₂O w/0.1% TFA) to afford 7-(4-isopropoxyphenyl)-3-(2-methylpyridin-4-yl)imidazo[1,2-a]pyridine (4.3 mg, 14% yield).

LCMS: R_{T}=0.544 min, >98% @ 215 nM and ELSD, m/z=344.1 [M+H]⁺.

TABLE 1
Mass Spectra data

| Example | Compound | Name | M + H |
|---|---|---|---|
| 1 | 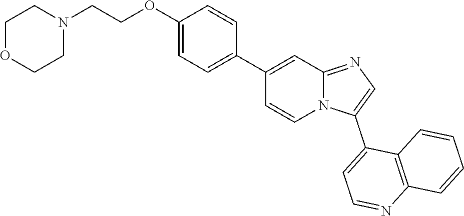 [SMILES: c1ccc2c(-c3cnc4cc(-c5ccc(OCCN6CCOCC6)cc5)ccn34)ccnc2c1] | 4-(2-(4-(3-(quinolin-4-yl)imidazo[1,2-a] pyridin-7-yl)phenoxy)ethyl)morpholine | 451 |
| 2 | 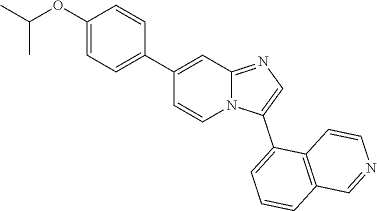 [SMILES: CC(C)Oc1ccc(-c2ccn3c(-c4cccc5cnccc45)cnc3c2)cc1] | 5-(7-(4-isopropoxyphenyl)imidazo[1,2-a] pyridin-3-yl)isoquinoline | 380 |
| 3 | 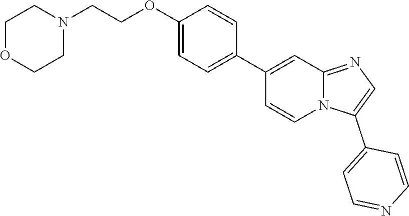 [SMILES: c1cc(-c2cnc3cc(-c4ccc(OCCN5CCOCC5)cc4)ccn23)ccn1] | 4-(2-(4-(3-(pyridin-4-yl)imidazo[1,2-a] pyridin-7-yl)phenoxy)ethyl)morpholine | 401 |
| 4 | 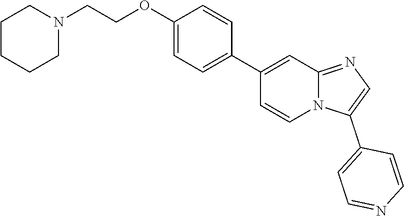 [SMILES: c1cc(-c2cnc3cc(-c4ccc(OCCN5CCCCC5)cc4)ccn23)ccn1] | 7-(4-(2-(piperidin-1-yl)ethoxy)phenyl)-3- (pyridin-4-yl)imidazo[1,2-a]pyridine | 399 |
| 5 | 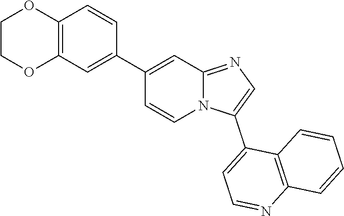 [SMILES: c1ccc2c(-c3cnc4cc(-c5ccc6c(c5)OCCO6)ccn34)ccnc2c1] | 4-(7-(2,3-dihydrobenzo[b][1,4]dioxin-6- yl)imidazo[1,2-a]pyridin-3-yl)quinoline | 380 |
| 6 | 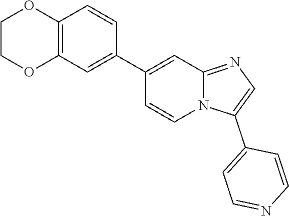 [SMILES: c1cc(-c2cnc3cc(-c4ccc5c(c4)OCCO5)ccn23)ccn1] | 7-(2,3-dihydrobenzo[b][1,4]dioxin-6-yl)-3- (pyridin-4-yl)imidazo[1,2-a]pyridine | 330 |
| 7 | 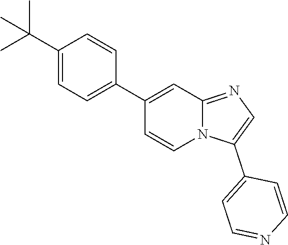 [SMILES: CC(C)(C)c1ccc(-c2ccn3c(-c4ccncc4)cnc3c2)cc1] | 7-(4-(tert-butyl)phenyl)-3-(pyridin-4- yl)imidazo[1,2-a]pyridine | 328 |
| 8 | 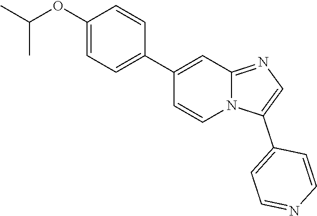 [SMILES: CC(C)Oc1ccc(-c2ccn3c(-c4ccncc4)cnc3c2)cc1] | 7-(4-isopropoxyphenyl)-3-(pyridin-4- yl)imidazo[1,2-a]pyridine | 330 |
| 9 | 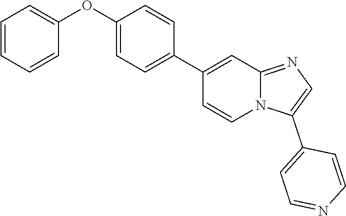 [SMILES: c1ccc(Oc2ccc(-c3ccn4c(-c5ccncc5)cnc4c3)cc2)cc1] | 7-(4-phenoxyphenyl)-3-(pyridin-4- yl)imidazo[1,2-a]pyridine | 364 |
| 10 | 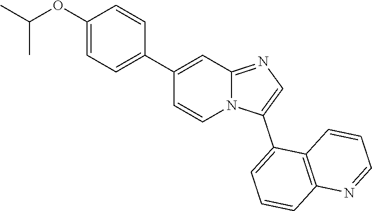 [SMILES: CC(C)Oc1ccc(-c2ccn3c(-c4cccc5ncccc45)cnc3c2)cc1] | 5-(7-(4-isopropoxyphenyl)imidazo[1,2-a] pyridin-3-yl)quinoline | 380 |
| 11 | 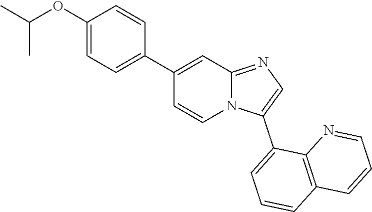 [SMILES: CC(C)Oc1ccc(-c2ccn3c(-c4cccc5cccnc45)cnc3c2)cc1] | 8-(7-(4-isopropoxyphenyl)imidazo[1,2-a] pyridin-3-yl)quinoline | 380 |
| 12 | 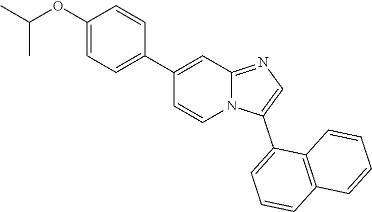 [SMILES: CC(C)Oc1ccc(-c2ccn3c(-c4cccc5ccccc45)cnc3c2)cc1] | 7-(4-isopropoxyphenyl)-3-(naphthalen-1- yl)imidazo[1,2-a]pyridine | 379 |
| 13 | 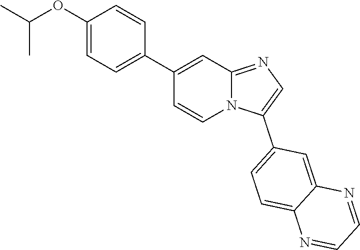 [SMILES: CC(C)Oc1ccc(-c2ccn3c(-c4ccc5nccnc5c4)cnc3c2)cc1] | 6-(7-(4-isopropoxyphenyl)imidazo[1,2-a] pyridin-3-yl)quinoxaline | 381 |
| 14 | 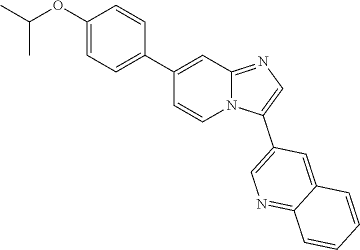 [SMILES: CC(C)Oc1ccc(-c2ccn3c(-c4cnc5ccccc5c4)cnc3c2)cc1] | 3-(7-(4-isopropoxyphenyl)imidazo[1,2-a] pyridin-3-yl)quinoline | 380 |
| 15 | 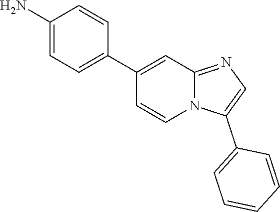 [SMILES: Nc1ccc(-c2ccn3c(-c4ccccc4)cnc3c2)cc1] | 4-(3-phenylimidazo[1,2-a]pyridin-7-yl) aniline | 286 |
| 16 | 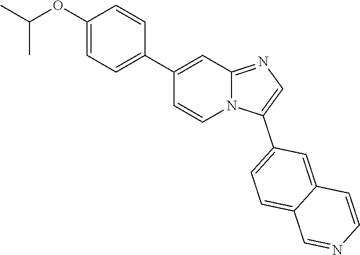 [SMILES: CC(C)Oc1ccc(-c2ccn3c(-c4ccc5cnccc5c4)cnc3c2)cc1] | 6-(7-(4-isopropoxyphenyl)imidazo[1,2-a] pyridin-3-yl)isoquinoline | 380 |
| 17 | 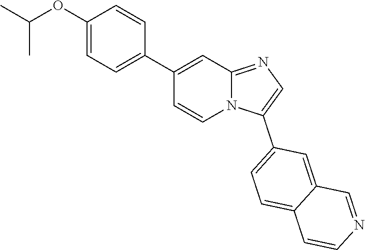 [SMILES: CC(C)Oc1ccc(-c2ccn3c(-c4ccc5ccncc5c4)cnc3c2)cc1] | 7-(7-(4-isopropoxyphenyl)imidazo[1,2-a] pyridin-3-yl)isoquinoline | 380 |
| 18 | 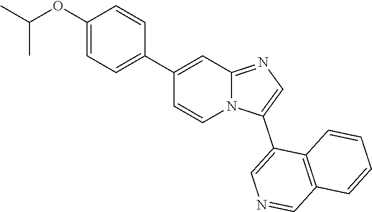 [SMILES: CC(C)Oc1ccc(-c2ccn3c(-c4cncc5ccccc45)cnc3c2)cc1] | 4-(7-(4-isopropoxyphenyl)imidazo[1,2-a] pyridin-3-yl)isoquinoline | 380 |
| 19 | 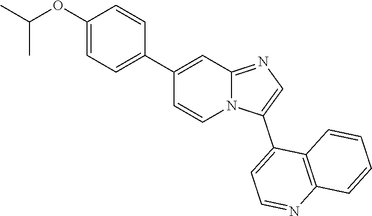 [SMILES: CC(C)Oc1ccc(-c2ccn3c(-c4ccnc5ccccc45)cnc3c2)cc1] | 4-(7-(4-isopropoxyphenyl)imidazo[1,2-a] pyridin-3-yl)quinoline | 380 |
| 20 | 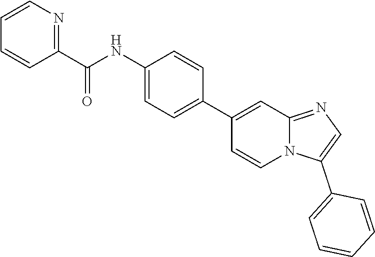 [SMILES: O=C(Nc1ccc(-c2ccn3c(-c4ccccc4)cnc3c2)cc1)c1ccccn1] | N-(4-(3-phenylimidazo[1,2-a]pyridin-7- yl)phenyl)picolinamide | 391 |
| 21 | 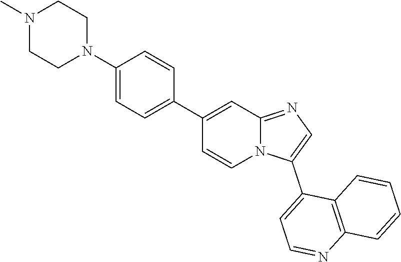 [SMILES: CN1CCN(c2ccc(-c3ccn4c(-c5ccnc6ccccc56)cnc4c3)cc2)CC1] | 4-(7-(4-(4-methylpiperazin-1-yl)phenyl) imidazo[1,2-a]pyridin-3-yl)quinoline | 420 |
| 22 | 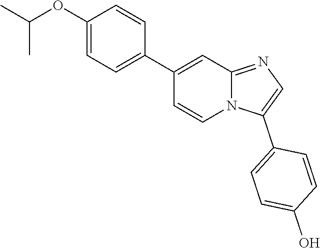 [SMILES: CC(C)Oc1ccc(-c2ccn3c(-c4ccc(O)cc4)cnc3c2)cc1] | 4-(7-(4-isopropoxyphenyl)imidazo[1,2-a] pyridin-3-yl)phenol | 345 |
| 23 | 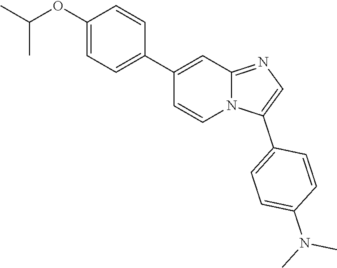 [SMILES: CC(C)Oc1ccc(-c2ccn3c(-c4ccc(N(C)C)cc4)cnc3c2)cc1] | 4-(7-(4-isopropoxyphenyl)imidazo[1,2-a] pyridin-3-yl)-N,N-dimethylaniline | 372 |
| 24 | 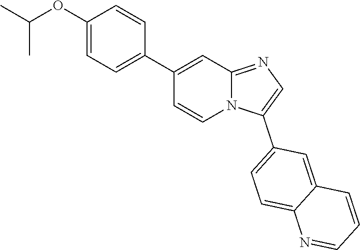 [SMILES: CC(C)Oc1ccc(-c2ccn3c(-c4ccc5ncccc5c4)cnc3c2)cc1] | 6-(7-(4-isopropoxyphenyl)imidazo[1,2-a] pyridin-3-yl)quinoline | 380 |
| 25 | 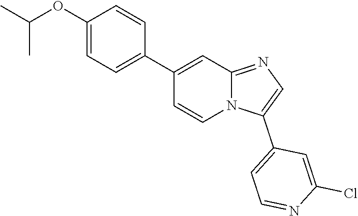 [SMILES: CC(C)Oc1ccc(-c2ccn3c(-c4ccnc(Cl)c4)cnc3c2)cc1] | 3-(2-chloropyridin-4-yl)-7-(4- isopropoxyphenyl)imidazo[1,2-a]pyridine | 364 |
| 26 | 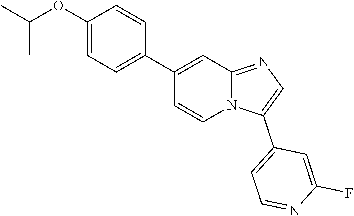 [SMILES: CC(C)Oc1ccc(-c2ccn3c(-c4ccnc(F)c4)cnc3c2)cc1] | 3-(2-fluoropyridin-4-yl)-7-(4- isopropoxyphenyl)imidazo[1,2-a]pyridine | 348 |
| 27 | 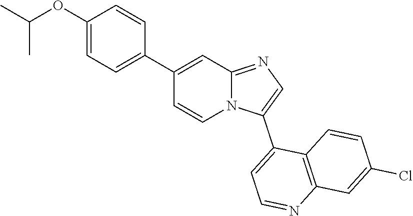 [SMILES: CC(C)Oc1ccc(-c2ccn3c(-c4ccnc5cc(Cl)ccc45)cnc3c2)cc1] | 7-chloro-4-(7-(4-isopropoxyphenyl)imidazo [1,2-a]pyridin-3-yl)quinoline | 414 |
| 28 | 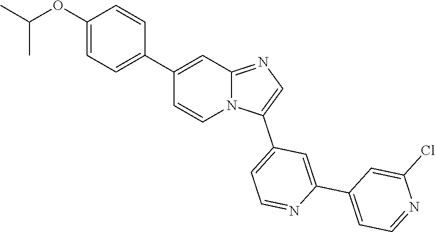 [SMILES: CC(C)Oc1ccc(-c2ccn3c(-c4ccnc(-c5ccnc(Cl)c5)c4)cnc3c2)cc1] | 3-(2′-chloro-[2,4′-bipyridin]-4-yl)-7-(4- isopropoxyphenyl)imidazo[1,2-a]pyridine | 441 |
| 29 | 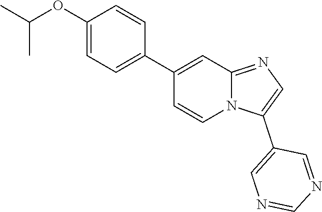 [SMILES: CC(C)Oc1ccc(-c2ccn3c(-c4cncnc4)cnc3c2)cc1] | 7-(4-isopropoxyphenyl)-3-(pyrimidin-5- yl)imidazo[1,2-a]pyridine | 331 |
| 30 | 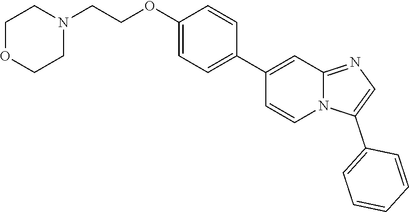 [SMILES: c1ccc(-c2cnc3cc(-c4ccc(OCCN5CCOCC5)cc4)ccn23)cc1] | 4-(2-(4-(3-phenylimidazo[1,2-a]pyridin-7- yl)phenoxy)ethyl)morpholine | 400 |
| 31 | 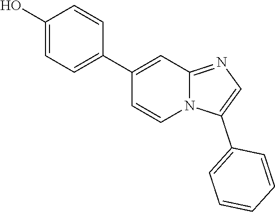 [SMILES: Oc1ccc(-c2ccn3c(-c4ccccc4)cnc3c2)cc1] | 4-(3-phenylimidazo[1,2-a]pyridin-7-yl)phenol | 287 |
| 32 | 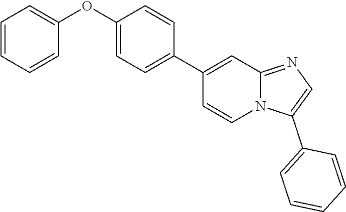 [SMILES: c1ccc(Oc2ccc(-c3ccn4c(-c5ccccc5)cnc4c3)cc2)cc1] | 7-(4-phenoxyphenyl)-3-phenylimidazo[1,2-a] pyridine | 363 |
| 33 | 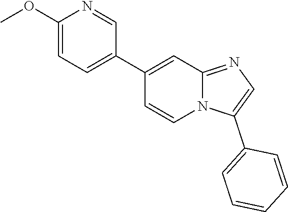 [SMILES: COc1ccc(-c2ccn3c(-c4ccccc4)cnc3c2)cn1] | 7-(6-methoxypyridin-3-yl)-3-phenylimidazo [1,2-a]pyridine | 302 |
| 34 | 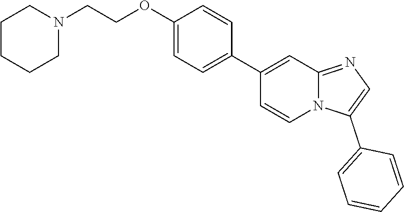 [SMILES: c1ccc(-c2cnc3cc(-c4ccc(OCCN5CCCCC5)cc4)ccn23)cc1] | 3-phenyl-7-(4-(2-(piperidin-1-yl)ethoxy) phenyl)imidazo[1,2-a]pyridine | 398 |
| 35 | 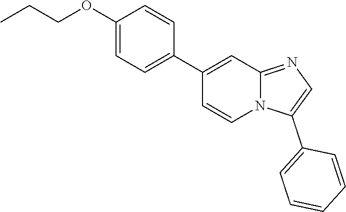 [SMILES: CCCOc1ccc(-c2ccn3c(-c4ccccc4)cnc3c2)cc1] | 3-phenyl-7-(4-propoxyphenyl)imidazo[1,2-a] pyridine | 329 |
| 36 | 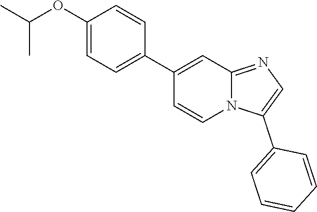 [SMILES: CC(C)Oc1ccc(-c2ccn3c(-c4ccccc4)cnc3c2)cc1] | 7-(4-isopropoxyphenyl)-3-phenylimidazo [1,2-a]pyridine | 329 |
| 37 | 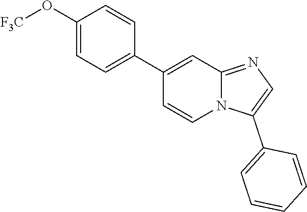 [SMILES: FC(F)(F)Oc1ccc(-c2ccn3c(-c4ccccc4)cnc3c2)cc1] | 3-phenyl-7-(4-(trifluoromethoxy)phenyl) imidazo[1,2-a]pyridine | 355 |
| 38 | 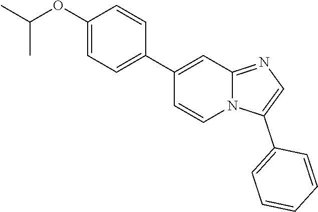 [SMILES: CC(C)Oc1ccc(-c2ccn3c(-c4ccccc4)cnc3c2)cc1] | 7-(4-isopropoxyphenyl)-3-phenylimidazo [1,2-a]pyridine | 329 |
| 39 | 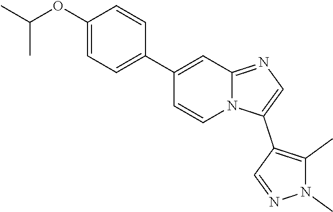 [SMILES: Cc1c(-c2cnc3cc(-c4ccc(OC(C)C)cc4)ccn23)cnn1C] | 3-(1,5-dimethyl-1H-pyrazol-4-yl)-7-(4- isopropoxyphenyl)imidazo[1,2-a]pyridine | 347 |
| 40 | 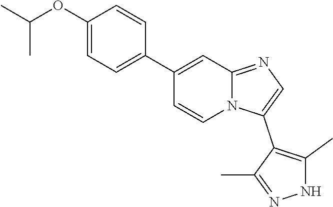 [SMILES: Cc1n[nH]c(C)c1-c1cnc2cc(-c3ccc(OC(C)C)cc3)ccn12] | 3-(3,5-dimethyl-1H-pyrazol-4-yl)-7-(4- isopropoxyphenyl)imidazo[1,2-a]pyridine | 347 |
| 41 | 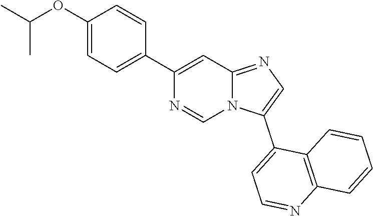 [SMILES: CC(C)Oc1ccc(-c2cc3ncc(-c4ccnc5ccccc45)n3cn2)cc1] | 4-(7-(4-isopropoxyphenyl)imidazo[1,2-c] pyrimidin-3-yl)quinoline | 381 |
| 42 | 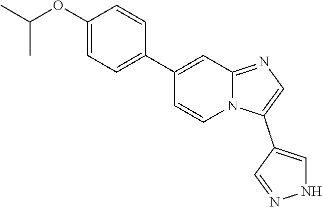 [SMILES: CC(C)Oc1ccc(-c2ccn3c(-c4cn[nH]c4)cnc3c2)cc1] | 7-(4-isopropoxyphenyl)-3-(1H-pyrazol-4- yl)imidazo[1,2-a]pyridine | 319 |
| 43 | 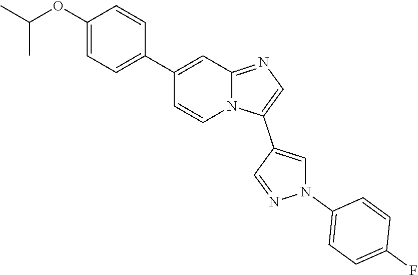 [SMILES: CC(C)Oc1ccc(-c2ccn3c(-c4cnn(-c5ccc(F)cc5)c4)cnc3c2)cc1] | 3-(1-(4-fluorophenyl)-1H-pyrazol-4-yl)-7-(4- isopropoxyphenyl)imidazo[1,2-a]pyridine | 413 |
| 44 | 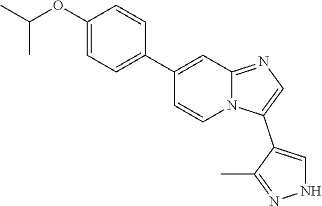 [SMILES: Cc1n[nH]cc1-c1cnc2cc(-c3ccc(OC(C)C)cc3)ccn12] | 7-(4-isopropoxyphenyl)-3-(3-methyl-1H- pyrazol-4-yl)imidazo[1,2-a]pyridine | 333 |
| 45 | 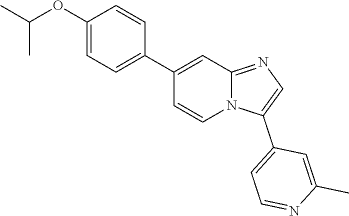 [SMILES: Cc1cc(-c2cnc3cc(-c4ccc(OC(C)C)cc4)ccn23)ccn1] | 7-(4-isopropoxyphenyl)-3-(2-methylpyridin-4- yl)imidazo[1,2-a]pyridine | 344 |
| 46 | 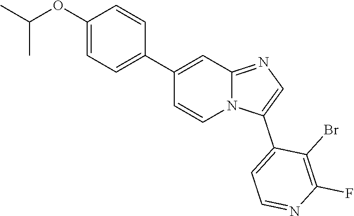 [SMILES: CC(C)Oc1ccc(-c2ccn3c(-c4ccnc(F)c4Br)cnc3c2)cc1] | 3-(3-bromo-2-fluoropyridin-4-yl)-7-(4- isopropoxyphenyl)imidazo[1,2-a]pyridine | 427 |
| 47 | 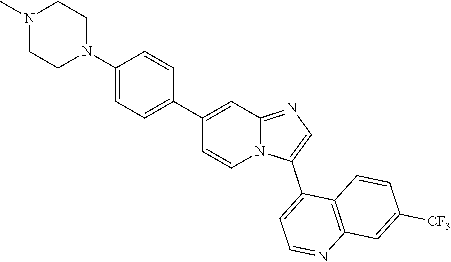 [SMILES: CN1CCN(c2ccc(-c3ccn4c(-c5ccnc6cc(C(F)(F)F)ccc56)cnc4c3)cc2)CC1] | 4-(7-(4-(4-methylpiperazin-1-yl)phenyl) imidazo[1,2-a]pyridin-3-yl)-7- (trifluoromethyl)quinoline | 488 |
| 48 | 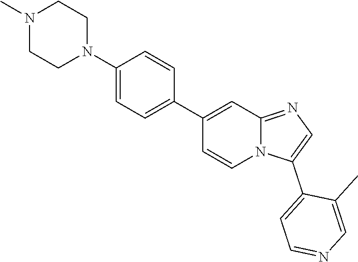 | 7-(4-(4-methylpiperazin-1-yl)phenyl)-3-(3- methylpyridin-4-yl)imidazo[1,2-a]pyridine | 384 |
| 49 | 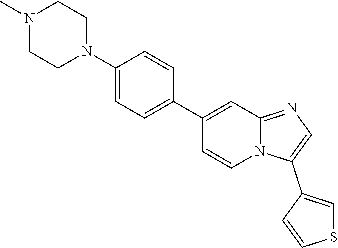 [SMILES: CN1CCN(c2ccc(-c3ccn4c(-c5ccsc5)cnc4c3)cc2)CC1] | 7-(4-(4-methylpiperazin-1-yl)phenyl)-3- (thiophen-3-yl)imidazo[1,2-a]pyridine | 375 |
| 50 | 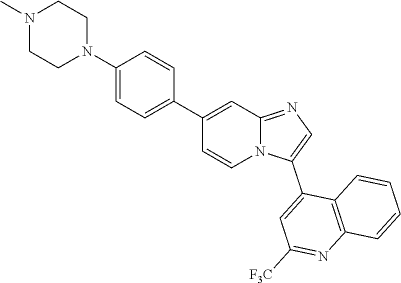 [SMILES: CN1CCN(c2ccc(-c3ccn4c(-c5cc(C(F)(F)F)nc6ccccc56)cnc4c3)cc2)CC1] | 4-(7-(4-(4-methylpiperazin-1- yl)phenyl)imidazo[1,2]pyridin-3-yl)-2- (trifluoromethyl)quinoline | 488 |
| 51 | 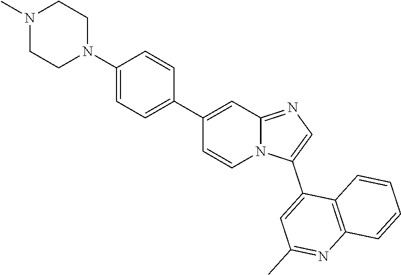 [SMILES: Cc1cc(-c2cnc3cc(-c4ccc(N5CCN(C)CC5)cc4)ccn23)c2ccccc2n1] | 2-methyl-4-(7-(4-(4-methylpiperazin-1- yl)phenyl)imidazo[1,2-a]pyridin-3-yl)quinoline | 434 |
| 52 | 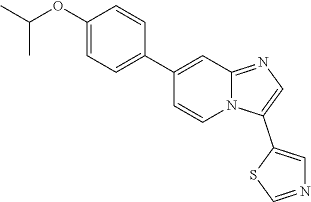 [SMILES: CC(C)Oc1ccc(-c2ccn3c(-c4cncs4)cnc3c2)cc1] | 5-(7-(4-isopropoxyphenyl)imidazo[1,2-a] pyridin-3-yl)thiazole | 336 |
| 53 | 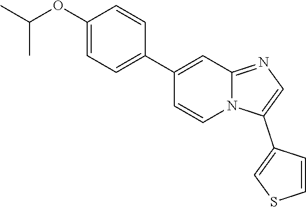 [SMILES: CC(C)Oc1ccc(-c2ccn3c(-c4ccsc4)cnc3c2)cc1] | 7-(4-isopropoxyphenyl)-3-(thiophen-3- yl)imidazo[1,2-a]pyridine | 335 |
| 54 | 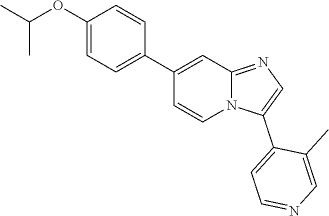 [SMILES: Cc1cnccc1-c1cnc2cc(-c3ccc(OC(C)C)cc3)ccn12] | 7-(4-isopropoxyphenyl)-3-(3-methylpyridin- 4-yl)imidazo[1,2-a]pyridine | 344 |
| 55 | 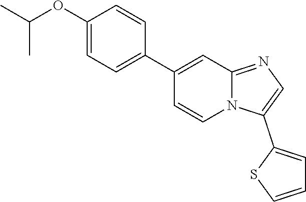 [SMILES: CC(C)Oc1ccc(-c2ccn3c(-c4cccs4)cnc3c2)cc1] | 7-(4-isopropoxyphenyl)-3-(thiophen-2- yl)imidazo[1,2-a]pyridine | 335 |
| 56 | 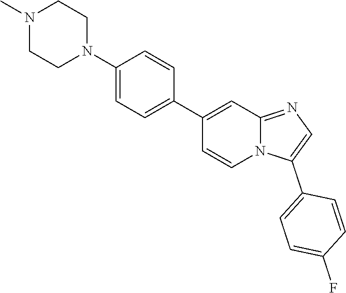 [SMILES: CN1CCN(c2ccc(-c3ccn4c(-c5ccc(F)cc5)cnc4c3)cc2)CC1] | 3-(4-fluorophenyl)-7-(4-(4-methylpiperazin- 1-yl)phenyl)imidazo[1,2-a]pyridine | 387 |
| 57 | 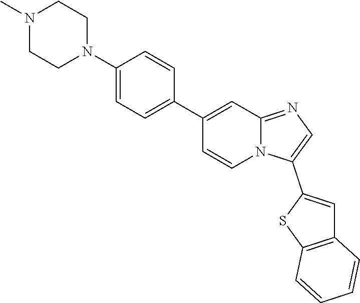 [SMILES: CN1CCN(c2ccc(-c3ccn4c(-c5cc6ccccc6s5)cnc4c3)cc2)CC1] | 3-(benzo[b]thiophen-2-yl)-7-(4-(4- methylpiperazin-1-yl)phenyl)imidazo[1,2-a] pyridine | 425 |
| 58 | 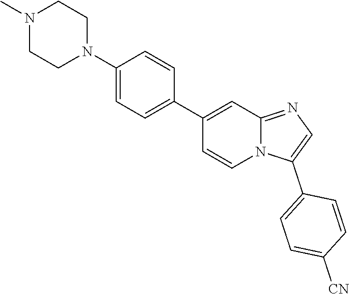 [SMILES: CN1CCN(c2ccc(-c3ccn4c(-c5ccc(C#N)cc5)cnc4c3)cc2)CC1] | 4-(7-(4-(4-methylpiperazin-1-yl)phenyl) imidazo[1,2-a]pyridin-3-yl)benzonitrile | 394 |
| 59 | 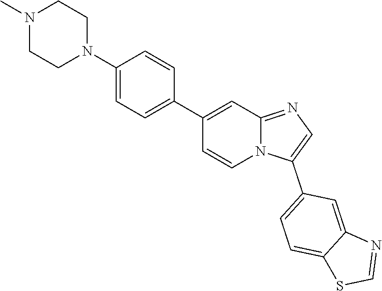 [SMILES: CN1CCN(c2ccc(-c3ccn4c(-c5ccc6scnc6c5)cnc4c3)cc2)CC1] | 5-(7-(4-(4-methylpiperazin-1- yl)phenyl)imidazo[1,2-a]pyridin-3- yl)benzo[d]thiazole | 426 |
| 60 | 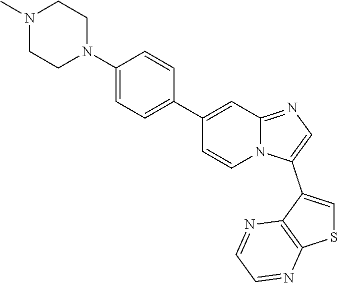 [SMILES: CN1CCN(c2ccc(-c3ccn4c(-c5csc6nccnc56)cnc4c3)cc2)CC1] | 7-(7-(4-(4-methylpiperazin-1- yl)phenyl)imidazo[1,2-a]pyridin-3-yl)thieno [2,3-b]pyrazine | 427 |
| 61 | 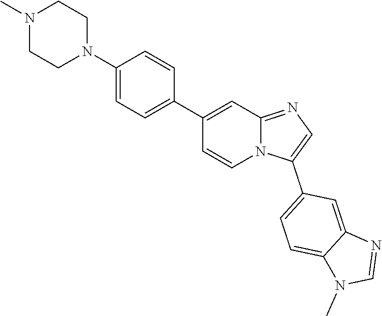 [SMILES: CN1CCN(c2ccc(-c3ccn4c(-c5ccc6c(c5)ncn6C)cnc4c3)cc2)CC1] | 1-methyl-5-(7-(4-(4-methylpiperazin-1- yl)phenyl)imidazo[1,2-a]pyridin-3-yl)-1H- benzo[d]imidazole | 423 |
| 62 | 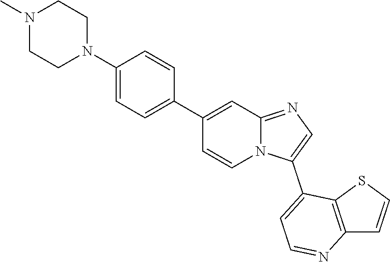 [SMILES: CN1CCN(c2ccc(-c3ccn4c(-c5ccnc6ccsc56)cnc4c3)cc2)CC1] | 7-(7-(4-(4-methylpiperazin-1- yl)phenyl)imidazo[1,2-a]pyridin-3-yl) thieno[3,2-b]pyridine | 426 |
| 63 | 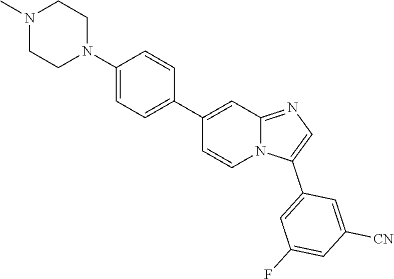 [SMILES: CN1CCN(c2ccc(-c3ccn4c(-c5cc(F)cc(C#N)c5)cnc4c3)cc2)CC1] | 3-fluoro-5-(7-(4-(4-methylpiperazin-1- yl)phenyl)imidazo[1,2-a]pyridin-3- yl)benzonitrile | 412 |
| 64 | 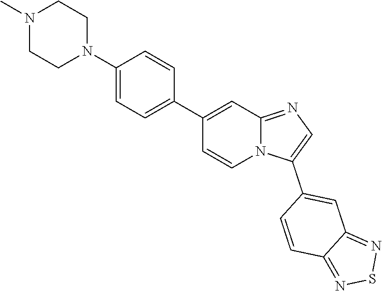 [SMILES: CN1CCN(c2ccc(-c3ccn4c(-c5ccc6nsnc6c5)cnc4c3)cc2)CC1] | 5-(7-(4-(4-methylpiperazin-1- yl)phenyl)imidazo[1,2-a]pyridin-3- yl)benzo[c][1,2,5]thiadiazole | 427 |
| 65 | 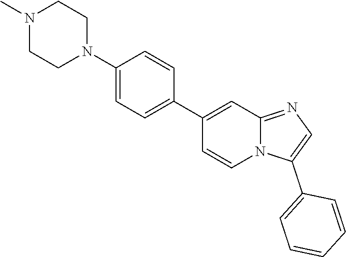 [SMILES: CN1CCN(c2ccc(-c3ccn4c(-c5ccccc5)cnc4c3)cc2)CC1] | 7-(4-(4-methylpiperazin-1-yl)phenyl)-3- phenylimidazo[1,2-a]pyridine | 369 |
| 66 | 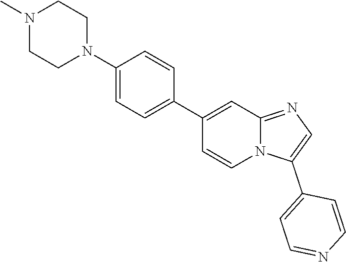 [SMILES: CN1CCN(c2ccc(-c3ccn4c(-c5ccncc5)cnc4c3)cc2)CC1] | 7-(4-(4-methylpiperazin-1-yl)phenyl)-3- (pyridin4-yl)imidazo[1,2-a]pyridine | 370 |
| 67 | 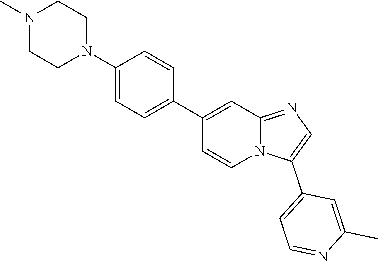 | 7-(4-(4-methylpiperazin-1-yl)phenyl)-3-(2- methylpyridin-4-yl)imidazo[1,2-a]pyridine | 384 |
| 68 | 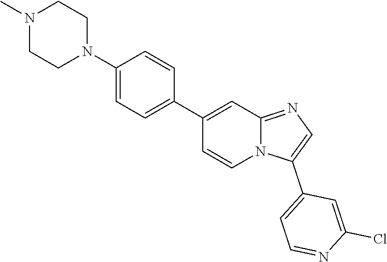 [SMILES: CN1CCN(c2ccc(-c3ccn4c(-c5ccnc(Cl)c5)cnc4c3)cc2)CC1] | 3-(2-chloropyridin-4-yl)-7-(4-(4- methylpiperazin-1-yl)phenyl)imidazo[1,2-a] pyridine | 404 |
| 69 | 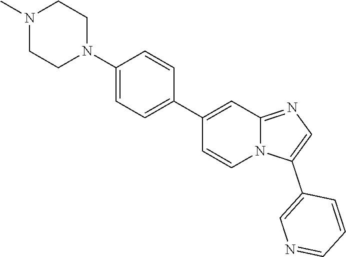 [SMILES: CN1CCN(c2ccc(-c3ccn4c(-c5cccnc5)cnc4c3)cc2)CC1] | 7-(4-(4-methylpiperazin-1-yl)phenyl)-3- (pyridin3-yl)imidazo[1,2-a]pyridine | 370 |
| 70 | 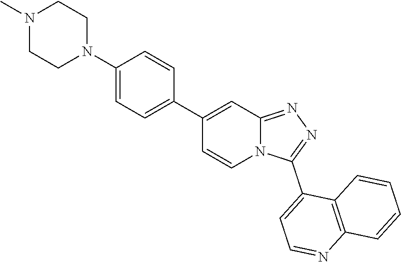 [SMILES: CN1CCN(c2ccc(-c3ccn4c(-c5ccnc6ccccc56)nnc4c3)cc2)CC1] | 4-(7-(4-(4-methylpiperazin-1-yl)phenyl)- [1,2,4]triazolo[4,3-a]pyridin-3-yl)quinoline | 421 |
| 71 | 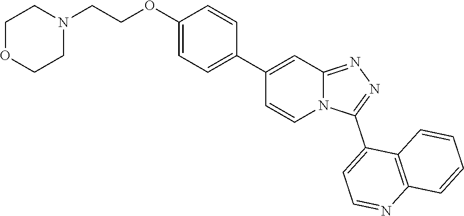 [SMILES: c1ccc2c(-c3nnc4cc(-c5ccc(OCCN6CCOCC6)cc5)ccn34)ccnc2c1] | 4-(2-(4-(3-(quinolin-4-yl)-[1,2,4]triazolo [4,3-a]pyridin-7-yl)phenoxy)ethyl)morpholine | 452 |
| 72 | 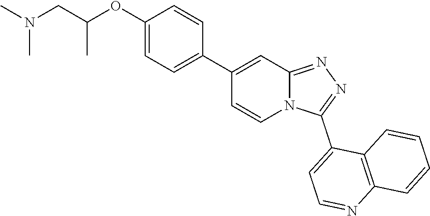 [SMILES: CC(CN(C)C)Oc1ccc(-c2ccn3c(-c4ccnc5ccccc45)nnc3c2)cc1] | N,N-dimethyl-2-(4-(3-(quinolin-4-yl)- [1,2,4]triazolo[4,3-a]pyridin-7- yl)phenoxy)propan-1-amine | 424 |
| 73 | 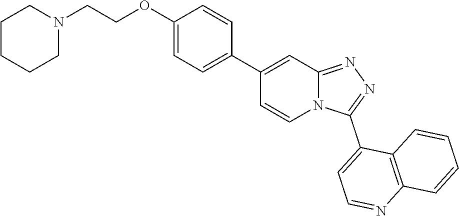 [SMILES: c1ccc2c(-c3nnc4cc(-c5ccc(OCCN6CCCCC6)cc5)ccn34)ccnc2c1] | 4-(7-(4-(2-(piperidin-1-yl)ethoxy)phenyl)- [1,2,4]triazolo[4,3-a]pyridin-3-yl)quinoline | 450 |
| 74 | 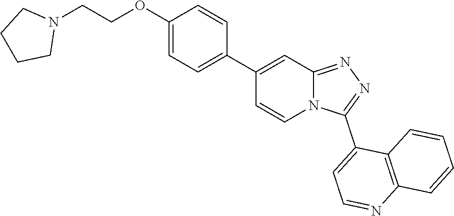 [SMILES: c1ccc2c(-c3nnc4cc(-c5ccc(OCCN6CCCC6)cc5)ccn34)ccnc2c1] | 4-(7-(4-(2-(pyrrolidin-1-yl)ethoxy)phenyl)- [1,2,4]triazolo[4,3-a]pyridin-3-yl)quinoline | 436 |
| 75 | 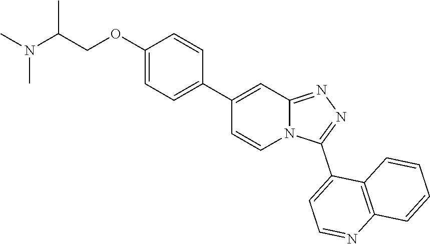 [SMILES: CC(COc1ccc(-c2ccn3c(-c4ccnc5ccccc45)nnc3c2)cc1)N(C)C] | N,N-dimethyl-1-(4-(3-(quinolin-4-yl)- [1,2,4]triazolo[4,3-a]pyridin-7-yl) phenoxy)propan-2-amine | 424 |
| 76 | 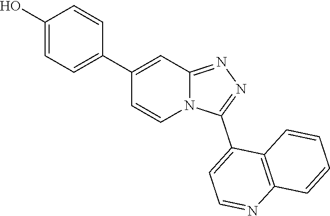 [SMILES: Oc1ccc(-c2ccn3c(-c4ccnc5ccccc45)nnc3c2)cc1] | 4-(3-(quinolin-4-yl)-[1,2,4]triazolo[4,3-a] pyridin-7-yl)phenol | 339 |
| 77 | 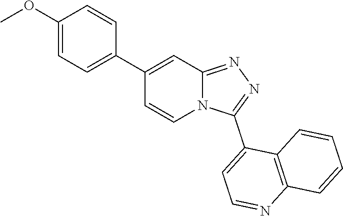 [SMILES: COc1ccc(-c2ccn3c(-c4ccnc5ccccc45)nnc3c2)cc1] | 4-(7-(4-methoxyphenyl)-[1,2,4]triazolo[4,3-a] pyridin-3-yl)quinoline | 353 |
| 78 | 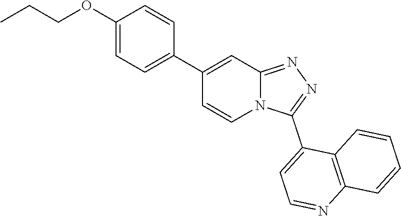 [SMILES: CCCOc1ccc(-c2ccn3c(-c4ccnc5ccccc45)nnc3c2)cc1] | 4-(7-(4-propoxyphenyl)-[1,2,4]triazolo[4,3-a] pyridin-3-yl)quinoline | 381 |
| 79 | 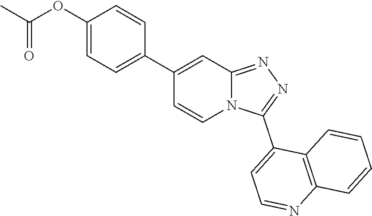 [SMILES: CC(=O)Oc1ccc(-c2ccn3c(-c4ccnc5ccccc45)nnc3c2)cc1] | 4-(3-(quinolin-4-yl)-[1,2,4]triazolo[4,3-a] pyridin-7-yl)phenyl acetate | 381 |
| 80 | 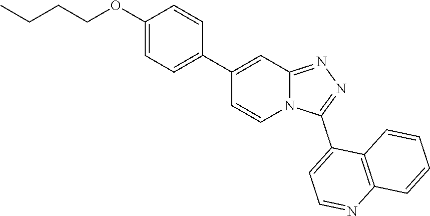 [SMILES: CCCCOc1ccc(-c2ccn3c(-c4ccnc5ccccc45)nnc3c2)cc1] | 4-(7-(4-butoxyphenyl)-[1,2,4]triazolo[4,3-a] pyridin-3-yl)quinoline | 395 |
| 81 | 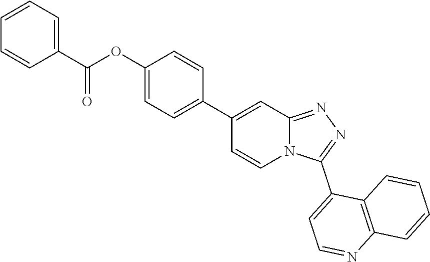 [SMILES: O=C(Oc1ccc(-c2ccn3c(-c4ccnc5ccccc45)nnc3c2)cc1)c1ccccc1] | 4-(3-(quinolin-4-yl)-[1,2,4]triazolo[4,3-a] pyridin-7-yl)phenyl benzoate | 443 |
| 82 | 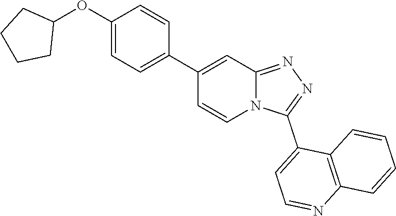 [SMILES: c1ccc2c(-c3nnc4cc(-c5ccc(OC6CCCC6)cc5)ccn34)ccnc2c1] | 4-(7-(4-(cyclopentyloxy)phenyl)- [1,2,4]triazolo[4,3-a]pyridin-3-yl)quinoline | 407 |
| 83 | 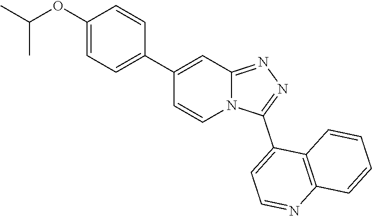 [SMILES: CC(C)Oc1ccc(-c2ccn3c(-c4ccnc5ccccc45)nnc3c2)cc1] | 4-(7-(4-isopropoxyphenyl)-[1,2,4]triazolo [4,3-a]pyridin-3-yl)quinoline | 381 |
| 84 | 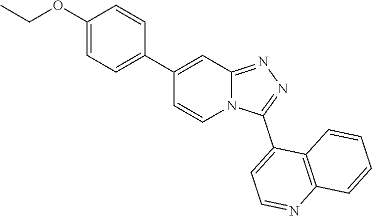 [SMILES: CCOc1ccc(-c2ccn3c(-c4ccnc5ccccc45)nnc3c2)cc1] | 4-(7-(4-cthoxyphenyl)-[1,2,4]triazolo[4,3-a] pyridin-3-yl)quinoline | 367 |
| 85 | 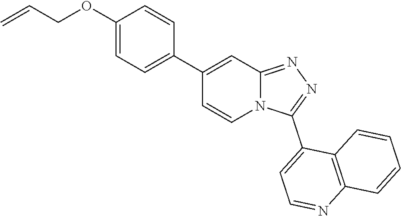 [SMILES: C=CCOc1ccc(-c2ccn3c(-c4ccnc5ccccc45)nnc3c2)cc1] | 4-(7-(4-(allyloxy)phenyl)-[1,2,4]triazolo [4,3-a]pyridin-3-yl)quinoline | 379 |
| 86 | 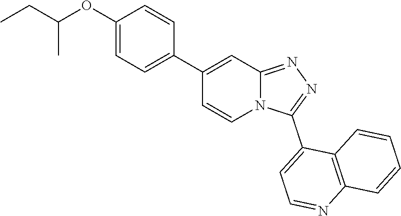 [SMILES: CCC(C)Oc1ccc(-c2ccn3c(-c4ccnc5ccccc45)nnc3c2)cc1] | 4-(7-(4-(sec-butoxy)phenyl)-[1,2,4]triazolo [4,3-a]pyridin-3-yl)quinoline | 395 |
| 87 | 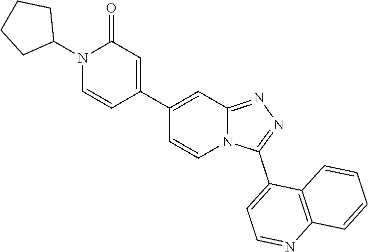 [SMILES: O=c1cc(-c2ccn3c(-c4ccnc5ccccc45)nnc3c2)ccn1C1CCCC1] | 1-cyclopentyl-4-(3-(quinolm-4-yl)-[1,2,4] triazolo[4,3-a]pyridin-7-yl)pyridin-2(1H)-one | 408 |
| 88 | 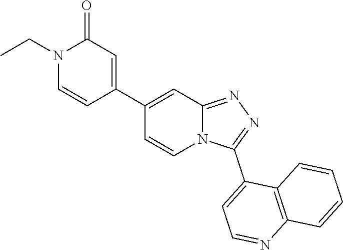 [SMILES: CCn1ccc(-c2ccn3c(-c4ccnc5ccccc45)nnc3c2)cc1=O] | 1-ethyl-4-(3-(quinolin-4-yl)-[1,2,4]triazolo [4,3-a]pyridin-7-yl)pyridin-2(1H)-one | 368 |
| 89 | 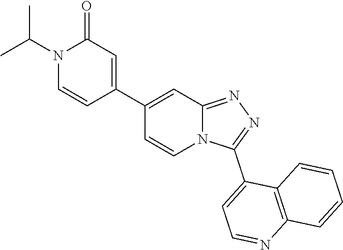 [SMILES: CC(C)n1ccc(-c2ccn3c(-c4ccnc5ccccc45)nnc3c2)cc1=O] | 1-isopropyl-4-(3-(quinolin-4-yl)-[1,2,4] triazolo[4,3-a]pyridin-7-yl)pyridin-2(1H)-one | 382 |
| 90 | 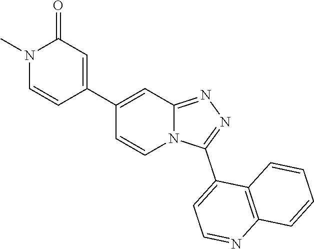 [SMILES: Cn1ccc(-c2ccn3c(-c4ccnc5ccccc45)nnc3c2)cc1=O] | 1-methyl-4-(3-(quinolin-4-yl)-[1,2,4] triazolo[4,3-a]pyridin-7-yl)pyridin-2(1H)-one | 354 |
| 91 | 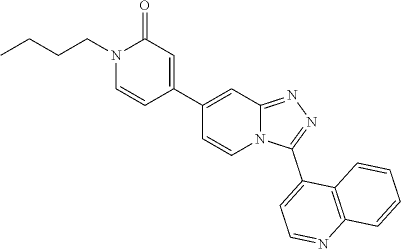 [SMILES: CCCCn1ccc(-c2ccn3c(-c4ccnc5ccccc45)nnc3c2)cc1=O] | 1-butyl-4-(3-(quinolin-4-yl)-[1,2,4]triazolo [4,3-a]pyridin-7-yl)pyridin-2(1H)-one | 396 |
| 92 | 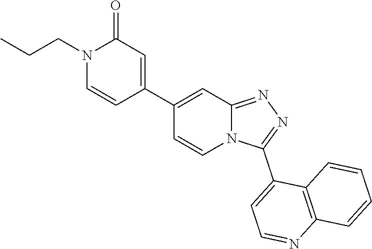 [SMILES: CCCn1ccc(-c2ccn3c(-c4ccnc5ccccc45)nnc3c2)cc1=O] | 1-propyl-4-(3-(quinolin-4-yl)-[1,2,4]triazolo [4.3-a]pyridin-7-yl)pyridin-2(1H)-one | 382 |
| 93 | 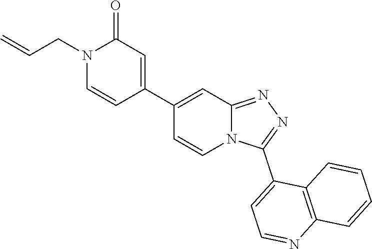 [SMILES: C=CCn1ccc(-c2ccn3c(-c4ccnc5ccccc45)nnc3c2)cc1=O] | 1-allyl-4-(3-(quinolin-4-yl)-[1,2,4]triazolo [4,3-a]pyridin-7-yl)pyridin-2(1H)-one | 380 |
| 94 | 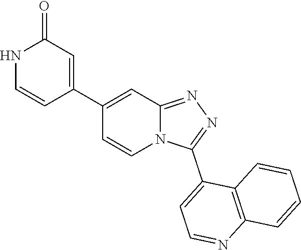 [SMILES: O=c1cc(-c2ccn3c(-c4ccnc5ccccc45)nnc3c2)cc[nH]1] | 4-(3-(quinolin-4-yl)-[1,2,4]triazolo[4,3-a] pyridin-7-yl)pyridin-2(1H)-one | 340 |
| 95 | 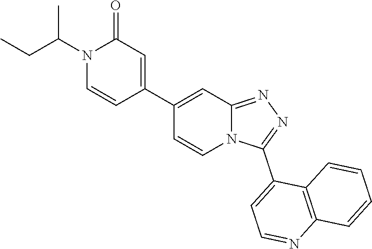 [SMILES: CCC(C)n1ccc(-c2ccn3c(-c4ccnc5ccccc45)nnc3c2)cc1=O] | 1-(sec-butyl)-4-(3-(quinolin-4-yl)-[1,2,4] triazolo[4,3-a]pyridin-7-yl)pyridin-2(1H)-one | 396 |

### Example 2: Biological Assays

Tables 2-4 summarize the results of assays used to identify and evaluate embodiments of the present invention.

TABLE 2

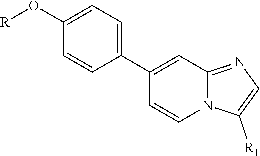

| | | | BMP4 |
|---|---|---|---|
| | | | Cell IC₅₀ |
| Example | R | R₁ | (nM) |
| 1 | 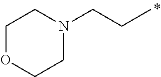 | 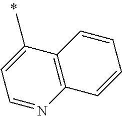 | 62 |
| 2 | | 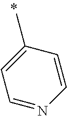 | |
| 3 | | 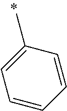 | |
| 4 | 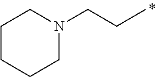 | 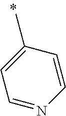 | |
| 5 | | 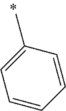 | |
| 6 | 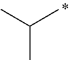 | 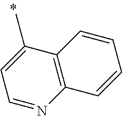 | 6,100 |
| 7 | | 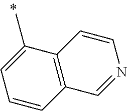 | |
| 8 | | 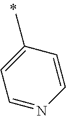 | |
| 9 | | 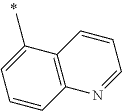 | |
| 10 | | 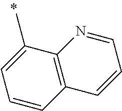 | >10,000 |
| 11 | | 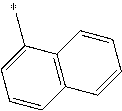 | |
| 12 | | 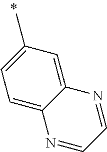 | Inactive |
| 13 | | 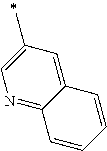 | Inactive |
| 14 | | 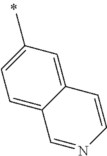 | Inactive |
| 15 | | 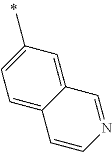 | Inactive |
| 16 | | 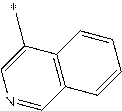 | Inactive |
| 17 | 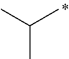 | 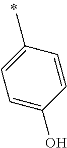 | Inactive |
| 18 | | 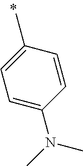 | Negative |
| 19 | | 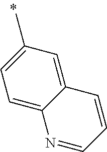 | Negative |
| 20 | | 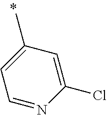 | 970 |
| 21 | | 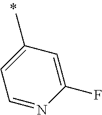 | 1,250 |
| 22 | | 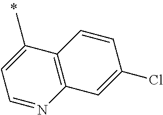 | >10,000 |
| 23 | | 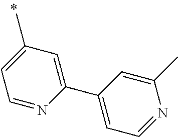 | Negative |
| 24 | | 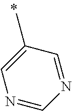 | Potentiates @ 1-10 uM |
| 25 | | 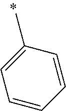 | Potentiates @ 1-10 uM |
| 26 | | 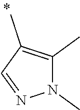 | Potentiates @ 0.5-10 uM |
| 27 | | 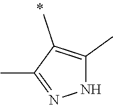 | Negative |
| 28 | | 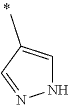 | 730 |
| 29 | | 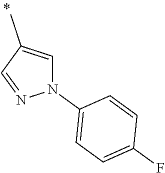 | inactive |
| 30 | | 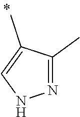 | >10,000 |
| 31 | | 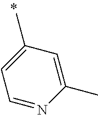 | 670 |
| 32 | | 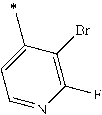 | Negative |
| 33 | | 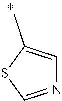 | >10,000 |
| 34 | | 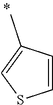 | Potentiates @ 0.1-10 uM |
| 35 | | 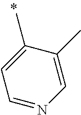 | Potentiates @ 1 uM but decrease @ 10 uM |
| 36 | | 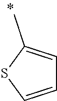 | Potentiates @ 0.1-10 uM |
| 37 | 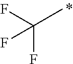 | 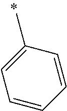 | Potentiates @ 1-10 uM |
| 38 | 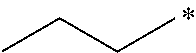 | 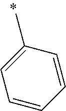 | Potentiates @ 0.5-10 uM |
| 39 | 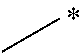 | 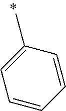 | Potentiates @ 0.1-10 uM |
| 40 | 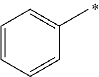 | 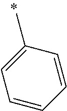 | Negative |
| 41 | 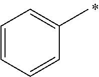 | 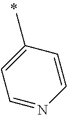 | |
| 42 | 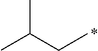 | 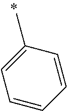 | |
| 43 | 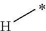 | 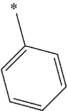 | |

| | |
|---|---|
| 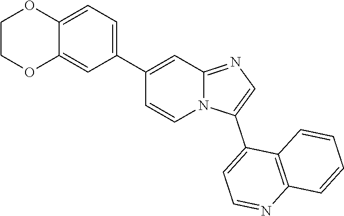 [SMILES: c1ccc2c(-c3cnc4cc(-c5ccc6c(c5)OCCO6)ccn34)ccnc2c1] | 2890 |
| 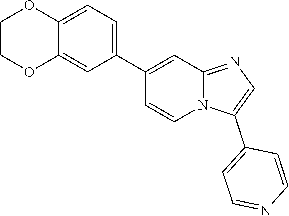 [SMILES: c1cc(-c2cnc3cc(-c4ccc5c(c4)OCCO5)ccn23)ccn1] | |
| 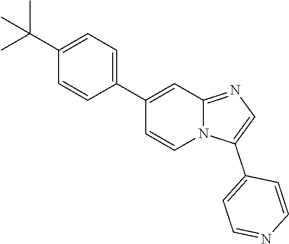 [SMILES: CC(C)(C)c1ccc(-c2ccn3c(-c4ccncc4)cnc3c2)cc1] | |
| 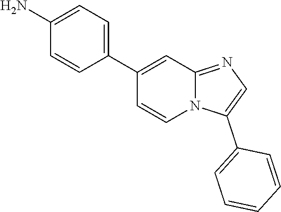 [SMILES: Nc1ccc(-c2ccn3c(-c4ccccc4)cnc3c2)cc1] | Actually induces/ potentiates at 0.5-10 uM (5-25-2012) |
| 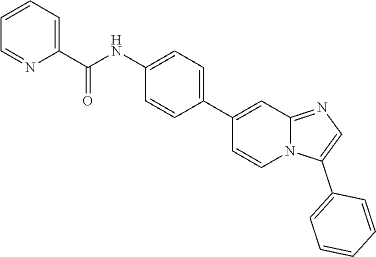 [SMILES: O=C(Nc1ccc(-c2ccn3c(-c4ccccc4)cnc3c2)cc1)c1ccccn1] | Actually induces/ potentiates BMP reproter (5-31-2012, set 9) |
| 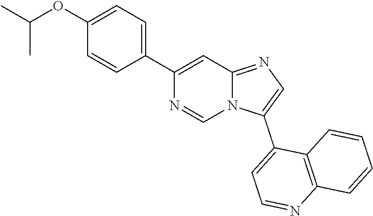 [SMILES: CC(C)Oc1ccc(-c2cc3ncc(-c4ccnc5ccccc45)n3cn2)cc1] | 8100 |

TABLE 3

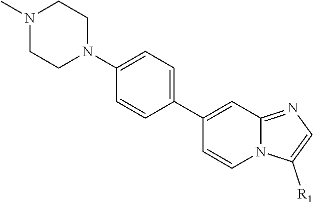

| | | BMP4 |
|---|---|---|
| | | Cell IC₅₀ |
| Example | R₁ | (nM) |
| 44 | 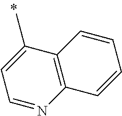 | <10 |
| 45 | 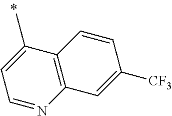 | >10,000 (TFA) |
| 46 | 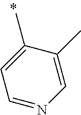 | 5,000 (TFA) |
| 47 | 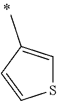 | 5,000 (TFA) |
| 48 | 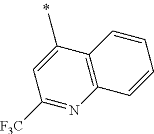 | inhibits at 10 uM |
| 49 | 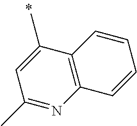 | 4.5 |
| 50 | 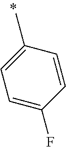 | Inhibits at 10 uM |
| 51 | 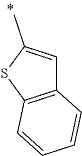 | No Inhibition to 10 uM |
| 52 | 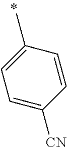 | PARTIAL INHIBITION |
| 53 | 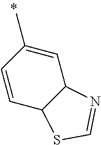 | Inhibits at 10 uM |
| 54 | 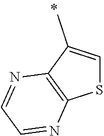 | 59 |
| 55 | 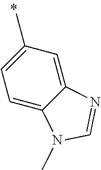 | No Inhibition to 10 uM |
| 56 | 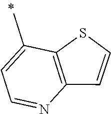 | 11.6 |
| 57 | 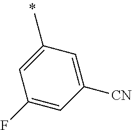 | 10,000 |
| 58 | 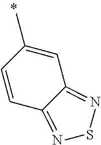 | No Inhibition to 10 uM. |
| 59 | 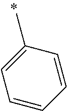 | Induces at 0.1- 1 uM. Partially inhibits @ 5 uM. KILLS at 10 uM |
| 60 | 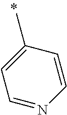 | 100 |
| 61 | 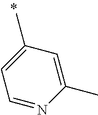 | 40 |
| 62 | 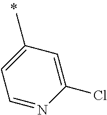 | 58 |
| 63 | 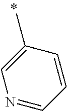 | Inhibits at 10 uM, but not at 5 uM |
| 64 | 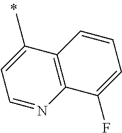 | <10 (TFA) |
| 65 | 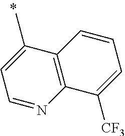 | Potent: Inhibits 50% @ 5 uM, fully @ 10 uM |
| 66 | 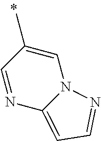 | Active at 10 uM |
| 67 | 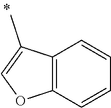 | Weakly Potent: Inhibits @ 5 uM, fully @ 10 uM (#26, 10-24-2012) |
| 68 | 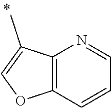 | Weakly Potent: Inhibits @ 5 uM, fully @ 10 uM (#26, 10-24-2012) |
| 69 | 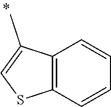 | 1280 (TFA) |
| 70 | 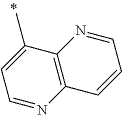 | <10 |
| 71 | 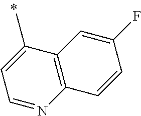 | 16.6 |
| 72 | 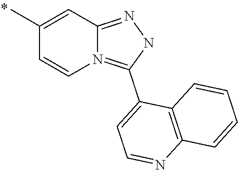 | inhibition at 10 uM |

TABLE 4

| | BMP Type 1 receptor (nM) | | | | | |
|---|---|---|---|---|---|---|
| | | ALK1/ | ALK3/ | ALK4/ | ALK5/ | |
| Entry | ALK2/ACVR1 | ACVR1 | BMPRR1A | ACVR1B | TGFBR1 | ALK6/BMPR1B |
| 1 | 53.1 | 49.6 | 52 | 31,000 | 23,000 | 151.2 |
| 5 | 270.0 | | | | | |
| 21 | 24.0 | 6.4 | 7.9 | ND | 3960 | 11 |
| 25 | 155.0 | 87 | 118 | ND | 13,600 | 340 |
| 26 | 265.0 | | | | >100,000 | |
| 30 | 645.1 | | | | >100,000 | |
| 42 | 1970.0 | 494 | 92 | ND | >100,000 | 895 |
| 45 | 149.0 | | | | 3490 | |
| 51 | 26.5 | | | | | |
| 60 | 1.3 | | 40.1 | | | |
| 62 | 14.4 | | 50.8 | | | |
| 66 | 46.0 | | | | | |
| 67 | 20.0 | | | | | |
| 68 | 33.2 | | 40.6 | | | |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating or preventing a disease or condition comprising administering to a subject a compound having the structure represented by Formula I: 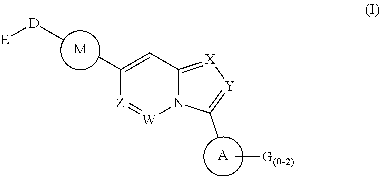
wherein:
W, X, Y, and Z are independently N or CH;
A is optionally substituted cycloalkyl, heterocyclyl, aryl or heteroaryl;
G is selected from H, CF₃, halogen, CN, alkyl, aryl, heteroaryl, NR¹R², CHR³R⁴, S(O)NR¹R², S(O)₂NR¹R², SR¹, SOR¹, or SO₂R¹;
M is optionally substituted aryl or heteroaryl;
D is selected from a bond, O, CR³R⁴, NR¹, NR¹R², SR¹, SOR¹, or SO₂R¹;
E is absent or selected from H, CF₃, halogen, CN, alkyl, aryl, heteroaryl, C₃-C₁₂ cycloalkyl, C₃-C₁₂ heterocyclyl, C₃-C₁₂ cycloalkylalkyl or C₃-C₁₂ heterocyclylalkyl;
R¹ is absent or selected from H, alkyl, aryl, or heteroaryl;
R² is selected from H, alkyl, aryl, heteroaryl, or COR¹, or
R₁ and R₂ form a C₃-C₁₂ cycloalkyl or C₃-C₁₂ heterocyclyl containing O, N and/or S;
R³ is selected from H, alkyl, aryl, or heteroaryl; and
R⁴ is selected from H, alkyl, aryl, heteroaryl, or COR¹, or
R³ and R⁴ form a C₃-C₁₂ cycloalkyl or a C₃-C₁₂ heterocyclyl containing O, N and/or S; or
a pharmaceutically acceptable salt thereof.

2. The method of claim 1, wherein W is CH.

3. The method of claim 1, wherein Z is CH.

4. The method of claim 1, wherein Z is N.

5. The method of claim 1, wherein X is N.

6. The method of claim 1, wherein Y is N.

7. The method of claim 1, wherein 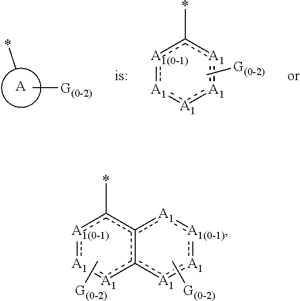
wherein each Ai is independently O, CR³R⁴, NH or NR¹, or can join with another A₁ to form C₃-C₁₂ cycloalkyl, C₃-C₁₂ cycloalkenyl, aryl, heteroaryl, or C₃-C₁₂ heterocyclyl.

8. The method of claim 1, wherein A is chosen from the following:

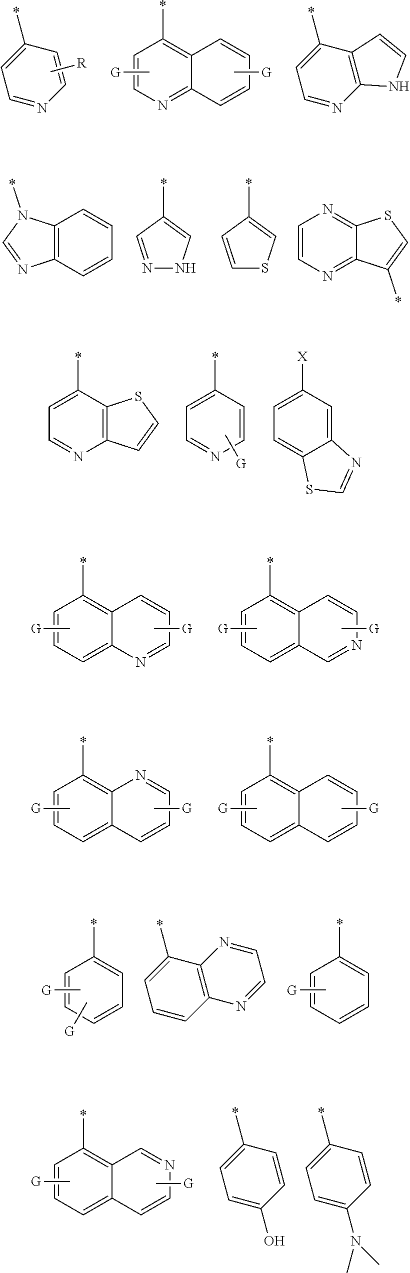

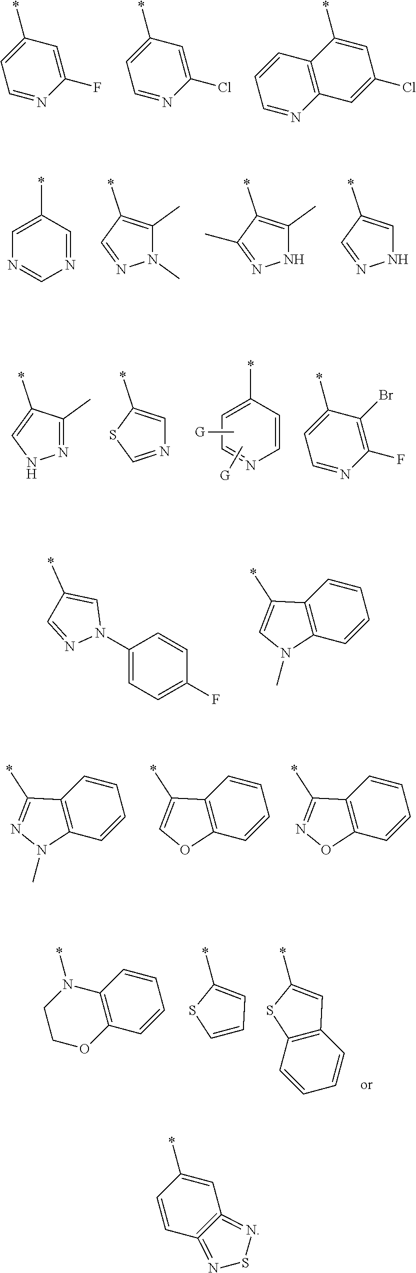

9. The method of claim 1, wherein M is optionally substituted with one or more G, and is selected from aryl or heteroaryl.

10. The method of claim 9, wherein M is optionally substituted phenyl or pyridine.

11. The method of claim 1, wherein M, D, and E together form:

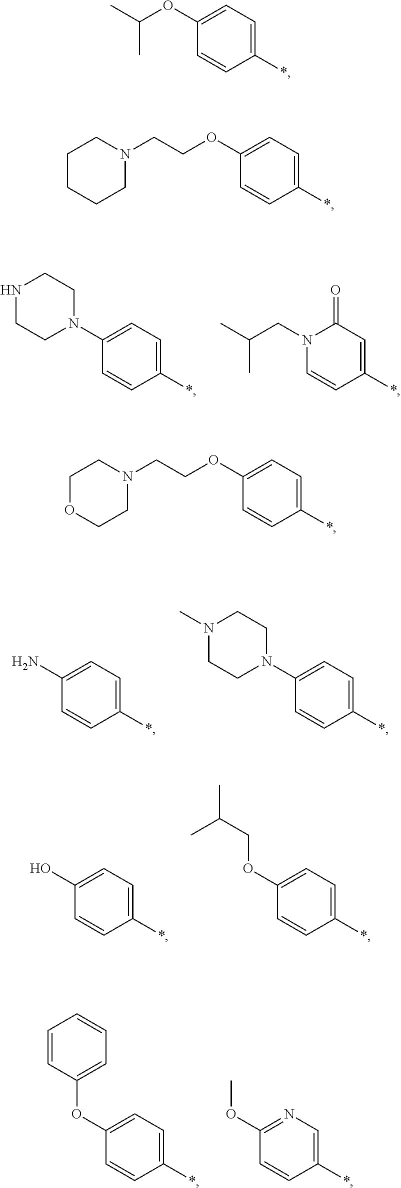

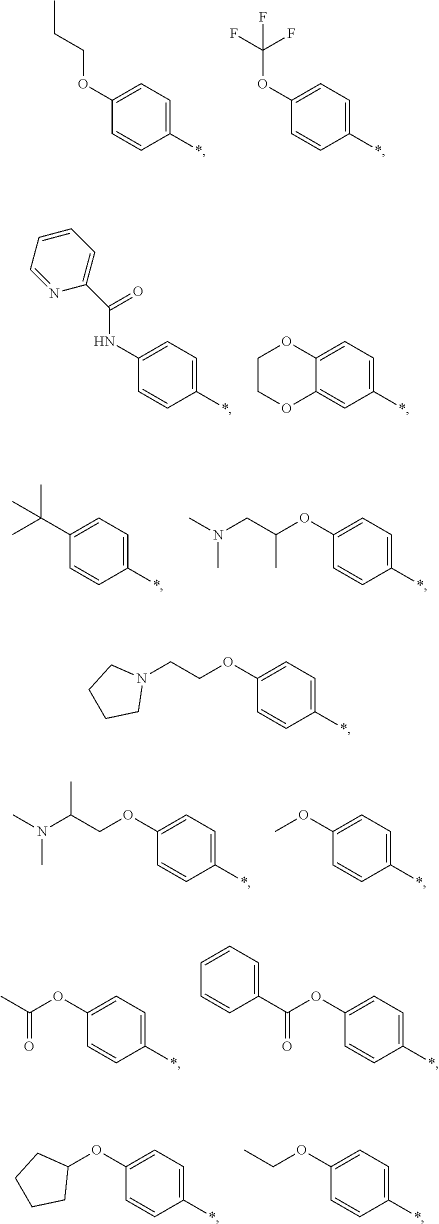

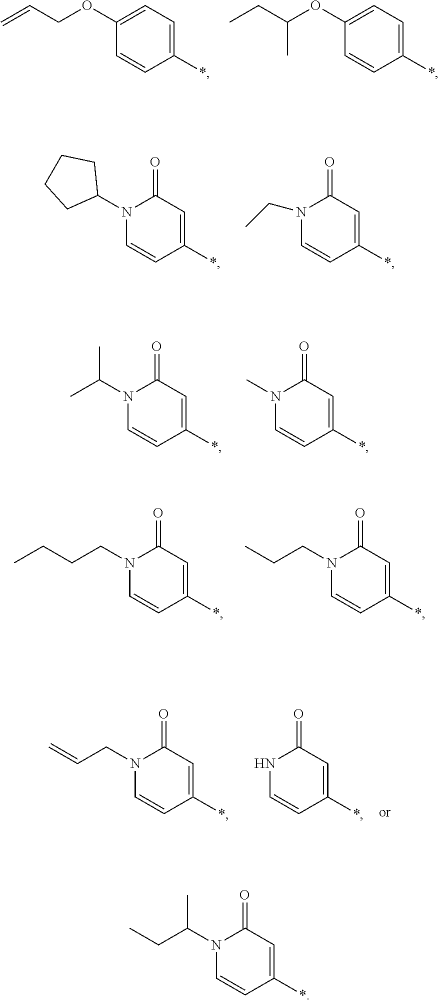

12. The method of claim 1, wherein the compound has the following structure: 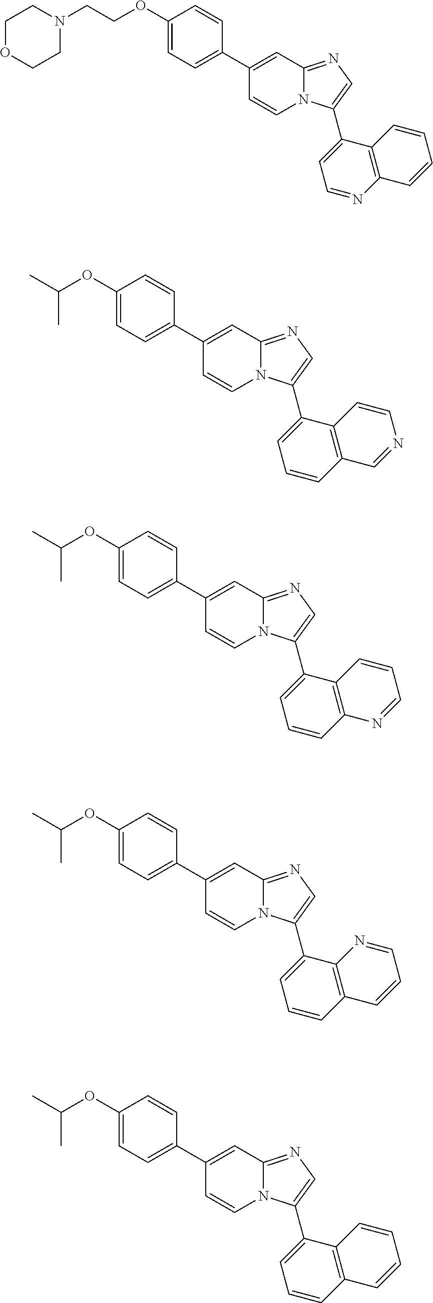 [SMILES: c1ccc2c(-c3cnc4cc(-c5ccc(OCCN6CCOCC6)cc5)ccn34)ccnc2c1 ; CC(C)Oc1ccc(-c2ccn3c(-c4cccc5cnccc45)cnc3c2)cc1 ; CC(C)Oc1ccc(-c2ccn3c(-c4cccc5ncccc45)cnc3c2)cc1 ; CC(C)Oc1ccc(-c2ccn3c(-c4cccc5cccnc45)cnc3c2)cc1 ; CC(C)Oc1ccc(-c2ccn3c(-c4cccc5ccccc45)cnc3c2)cc1] 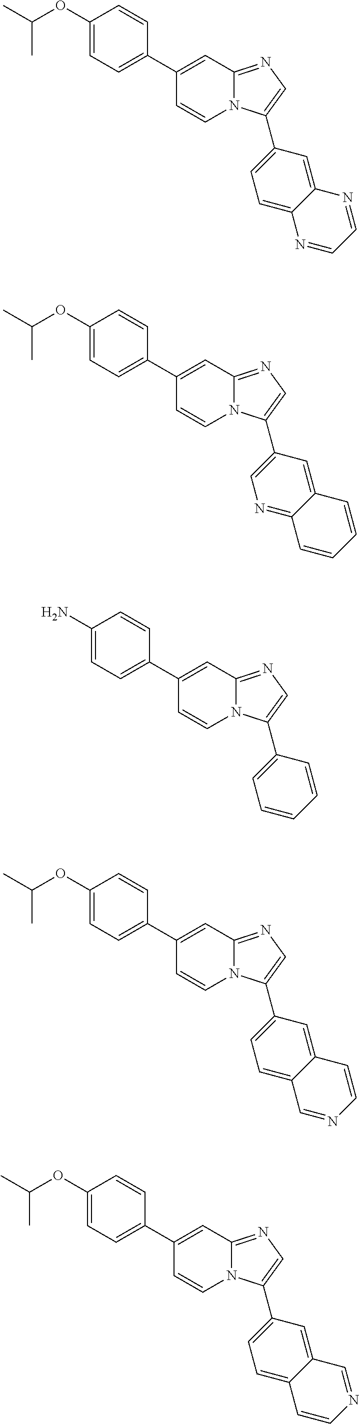 [SMILES: CC(C)Oc1ccc(-c2ccn3c(-c4ccc5nccnc5c4)cnc3c2)cc1 ; CC(C)Oc1ccc(-c2ccn3c(-c4cnc5ccccc5c4)cnc3c2)cc1 ; Nc1ccc(-c2ccn3c(-c4ccccc4)cnc3c2)cc1 ; CC(C)Oc1ccc(-c2ccn3c(-c4ccc5cnccc5c4)cnc3c2)cc1 ; CC(C)Oc1ccc(-c2ccn3c(-c4ccc5ccncc5c4)cnc3c2)cc1] 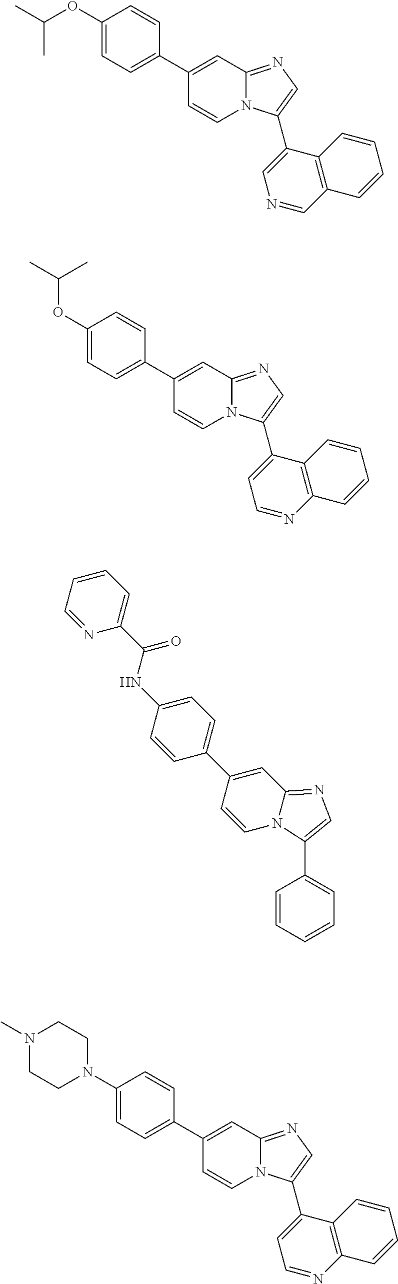 [SMILES: CC(C)Oc1ccc(-c2ccn3c(-c4cncc5ccccc45)cnc3c2)cc1 ; CC(C)Oc1ccc(-c2ccn3c(-c4ccnc5ccccc45)cnc3c2)cc1 ; O=C(Nc1ccc(-c2ccn3c(-c4ccccc4)cnc3c2)cc1)c1ccccn1 ; CN1CCN(c2ccc(-c3ccn4c(-c5ccnc6ccccc56)cnc4c3)cc2)CC1] 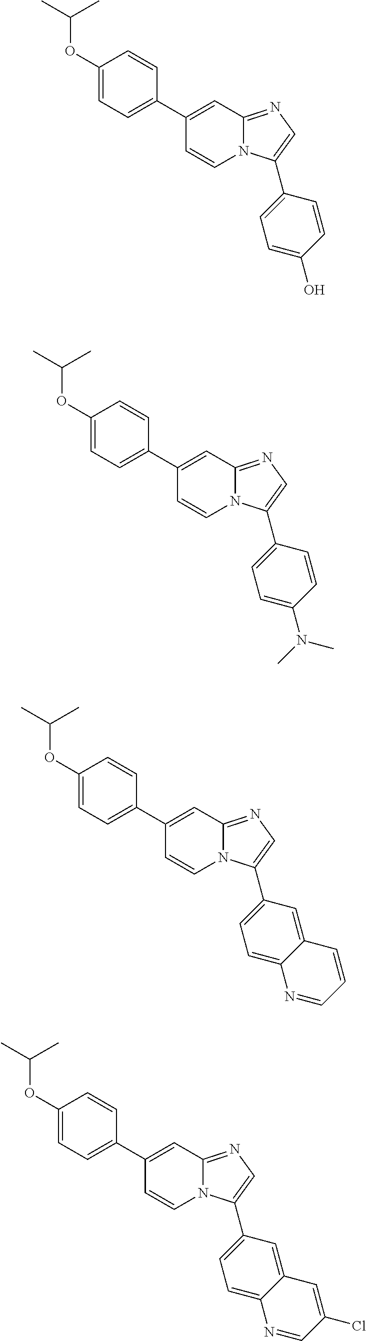 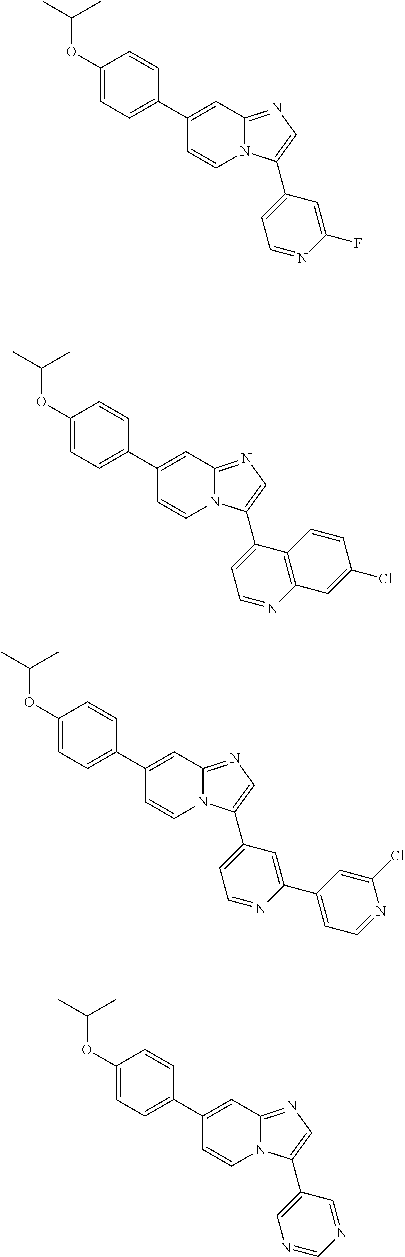 [SMILES: CC(C)Oc1ccc(-c2ccn3c(-c4ccnc(F)c4)cnc3c2)cc1 ; CC(C)Oc1ccc(-c2ccn3c(-c4ccnc5cc(Cl)ccc45)cnc3c2)cc1 ; CC(C)Oc1ccc(-c2ccn3c(-c4ccnc(-c5ccnc(Cl)c5)c4)cnc3c2)cc1 ; CC(C)Oc1ccc(-c2ccn3c(-c4cncnc4)cnc3c2)cc1] 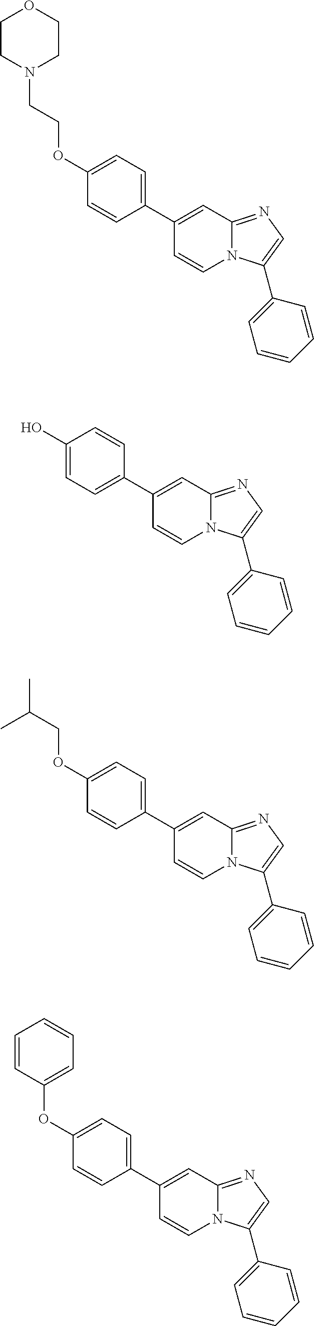 [SMILES: c1ccc(-c2cnc3cc(-c4ccc(OCCN5CCOCC5)cc4)ccn23)cc1 ; Oc1ccc(-c2ccn3c(-c4ccccc4)cnc3c2)cc1 ; CC(C)COc1ccc(-c2ccn3c(-c4ccccc4)cnc3c2)cc1 ; c1ccc(Oc2ccc(-c3ccn4c(-c5ccccc5)cnc4c3)cc2)cc1] 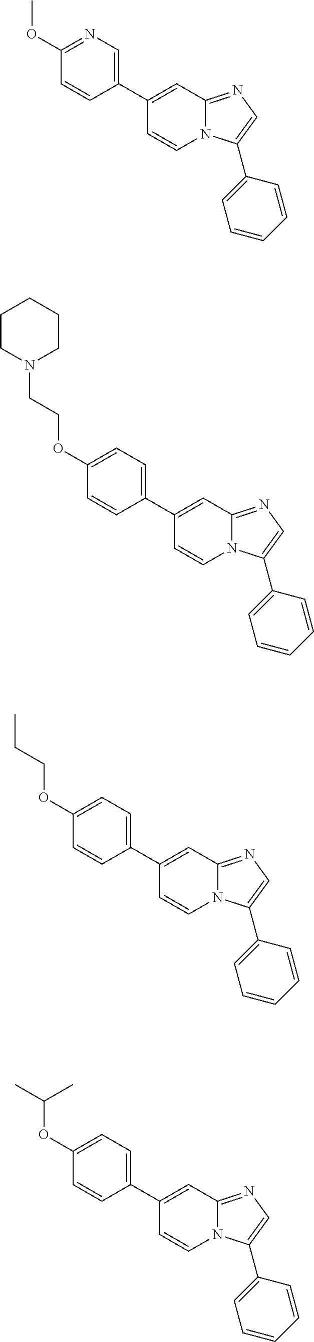 [SMILES: COc1ccc(-c2ccn3c(-c4ccccc4)cnc3c2)cn1 ; c1ccc(-c2cnc3cc(-c4ccc(OCCN5CCCCC5)cc4)ccn23)cc1 ; CCCOc1ccc(-c2ccn3c(-c4ccccc4)cnc3c2)cc1 ; CC(C)Oc1ccc(-c2ccn3c(-c4ccccc4)cnc3c2)cc1] 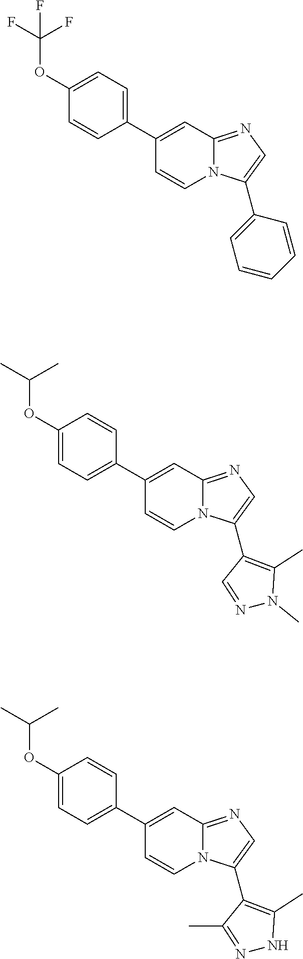 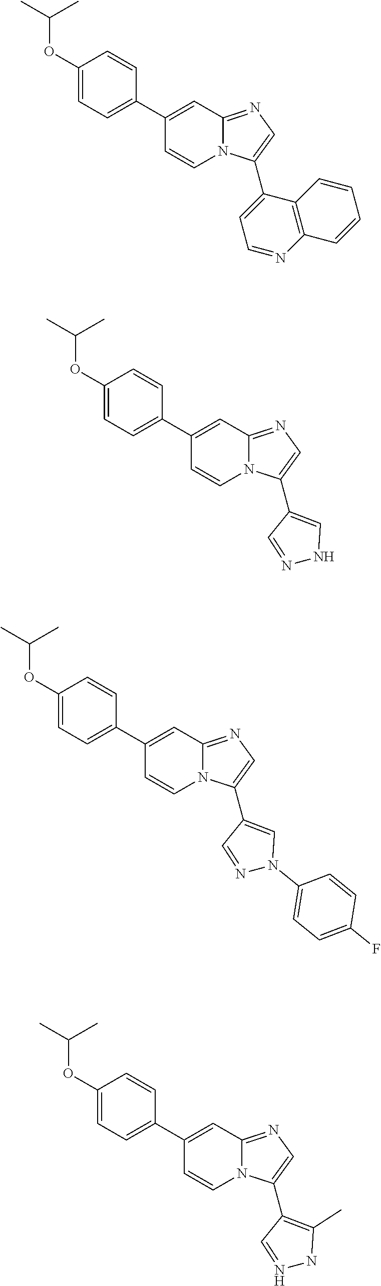 [SMILES: CC(C)Oc1ccc(-c2ccn3c(-c4ccnc5ccccc45)cnc3c2)cc1 ; CC(C)Oc1ccc(-c2ccn3c(-c4cn[nH]c4)cnc3c2)cc1 ; CC(C)Oc1ccc(-c2ccn3c(-c4cnn(-c5ccc(F)cc5)c4)cnc3c2)cc1 ; Cc1[nH]ncc1-c1cnc2cc(-c3ccc(OC(C)C)cc3)ccn12] 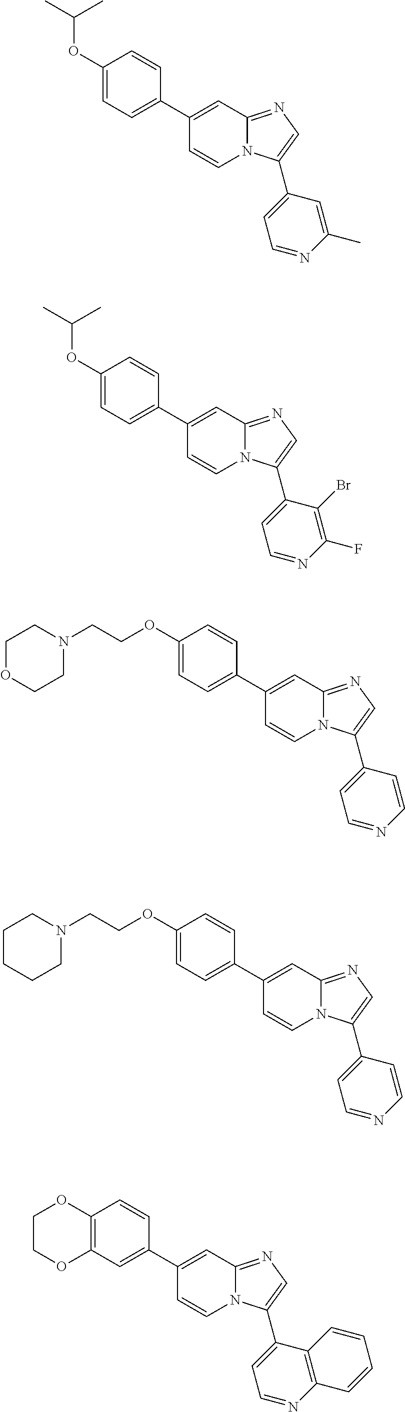 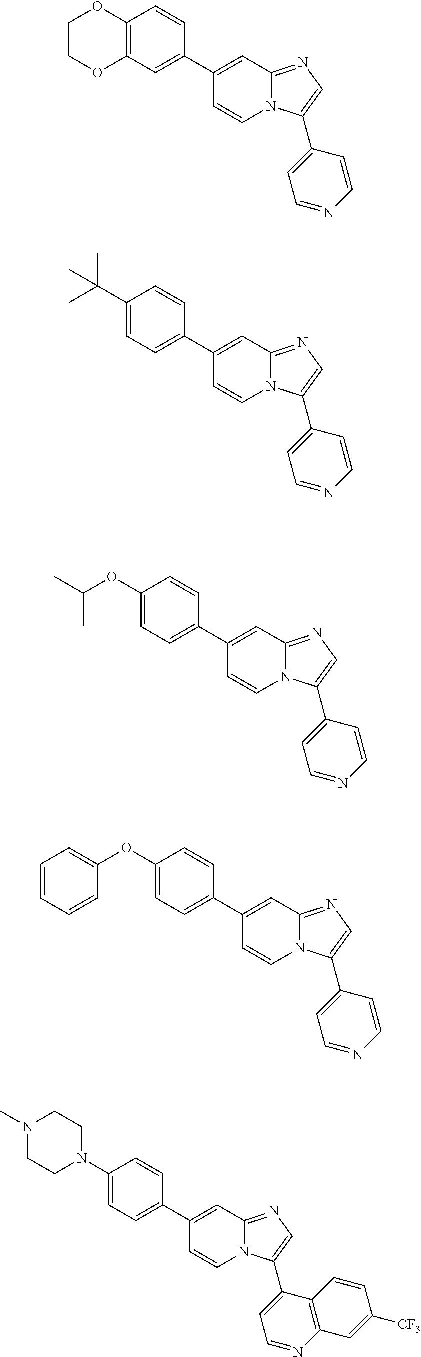 [SMILES: c1cc(-c2cnc3cc(-c4ccc5c(c4)OCCO5)ccn23)ccn1 ; CC(C)(C)c1ccc(-c2ccn3c(-c4ccncc4)cnc3c2)cc1 ; CC(C)Oc1ccc(-c2ccn3c(-c4ccncc4)cnc3c2)cc1 ; c1ccc(Oc2ccc(-c3ccn4c(-c5ccncc5)cnc4c3)cc2)cc1 ; CN1CCN(c2ccc(-c3ccn4c(-c5ccnc6cc(C(F)(F)F)ccc56)cnc4c3)cc2)CC1] 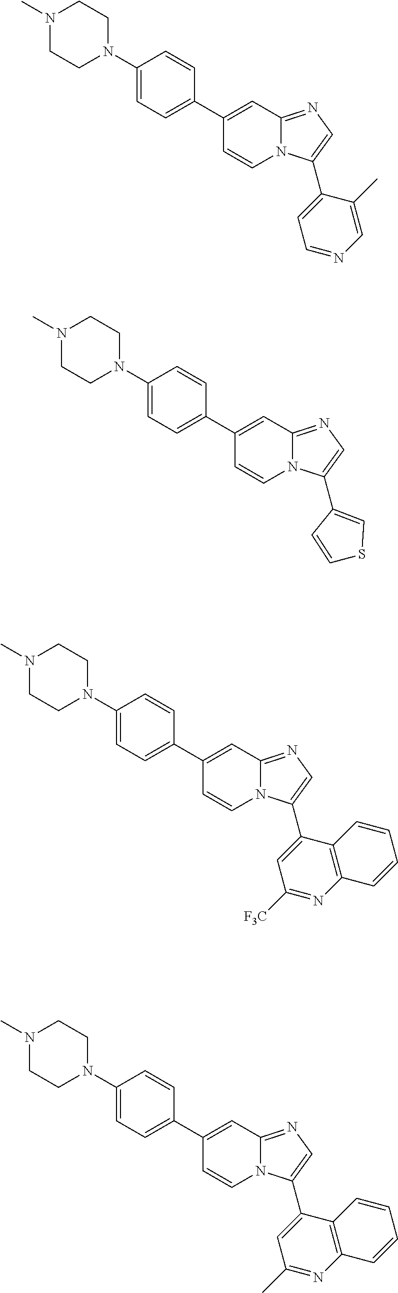 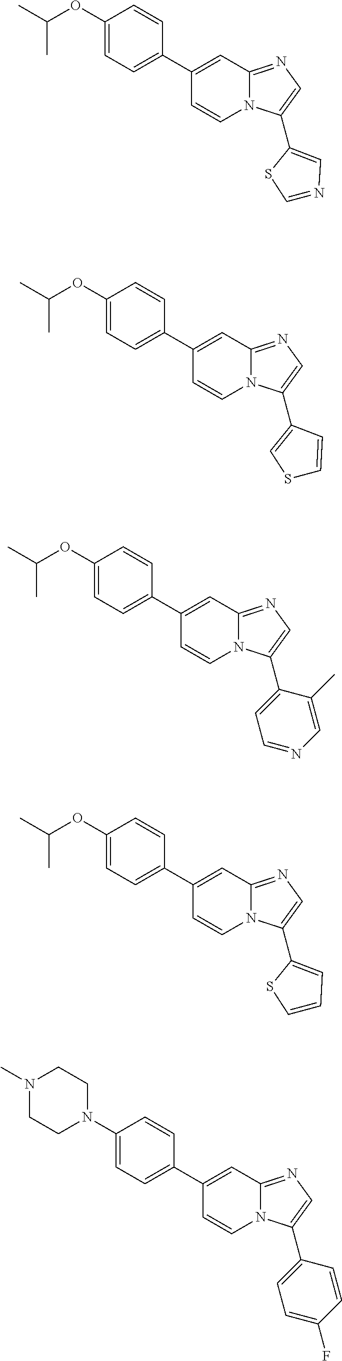 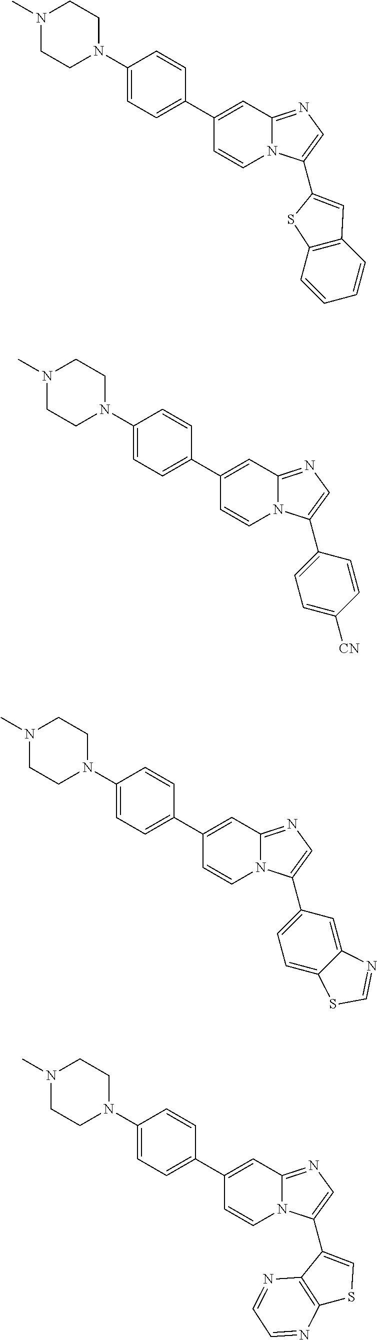 [SMILES: CN1CCN(c2ccc(-c3ccn4c(-c5cc6ccccc6s5)cnc4c3)cc2)CC1 ; CN1CCN(c2ccc(-c3ccn4c(-c5ccc(C#N)cc5)cnc4c3)cc2)CC1 ; CN1CCN(c2ccc(-c3ccn4c(-c5ccc6scnc6c5)cnc4c3)cc2)CC1 ; CN1CCN(c2ccc(-c3ccn4c(-c5csc6nccnc56)cnc4c3)cc2)CC1] 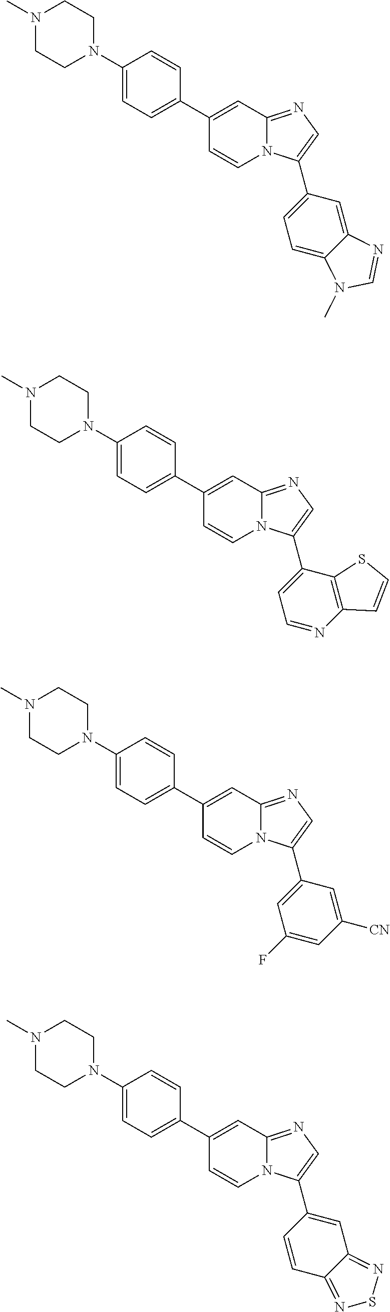 [SMILES: CN1CCN(c2ccc(-c3ccn4c(-c5ccc6c(c5)ncn6C)cnc4c3)cc2)CC1 ; CN1CCN(c2ccc(-c3ccn4c(-c5ccnc6ccsc56)cnc4c3)cc2)CC1 ; CN1CCN(c2ccc(-c3ccn4c(-c5cc(F)cc(C#N)c5)cnc4c3)cc2)CC1 ; CN1CCN(c2ccc(-c3ccn4c(-c5ccc6nsnc6c5)cnc4c3)cc2)CC1] 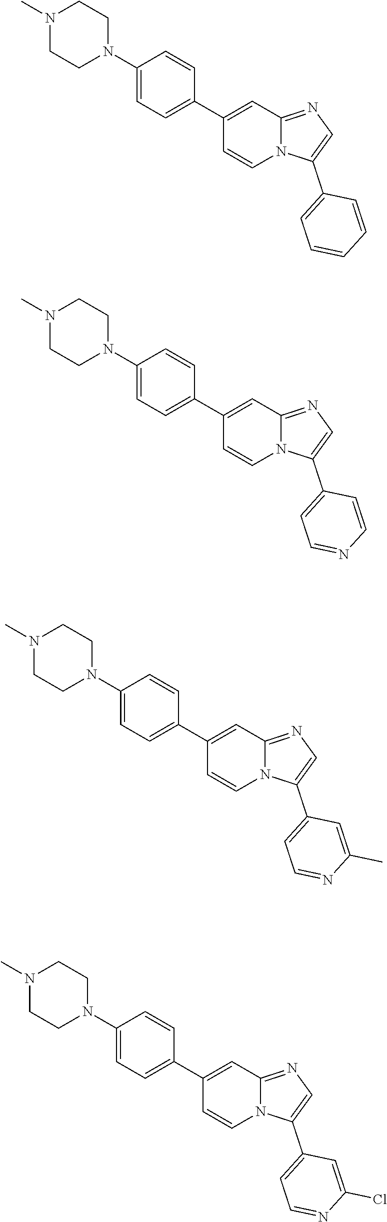 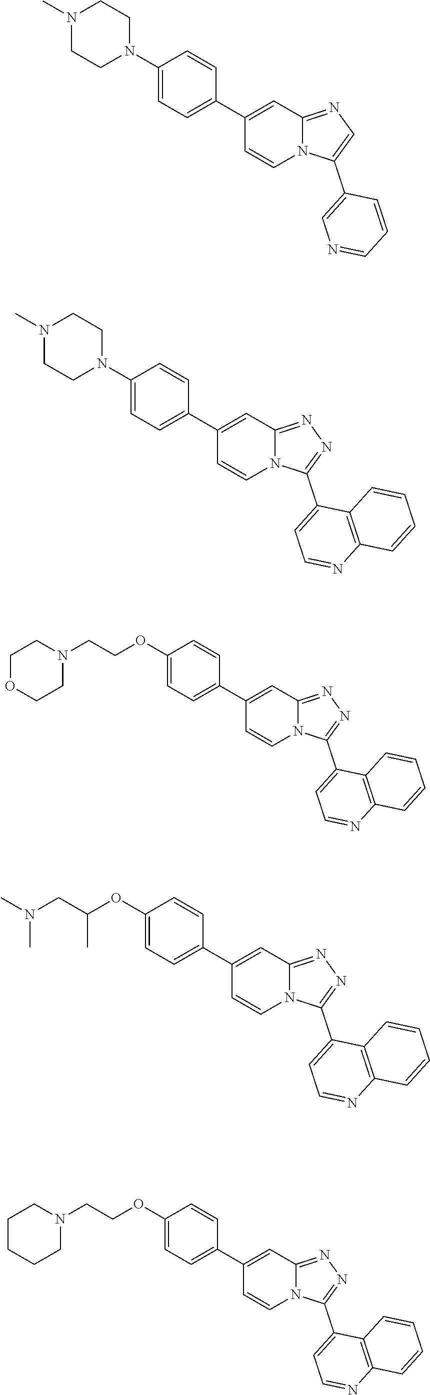 [SMILES: CN1CCN(c2ccc(-c3ccn4c(-c5cccnc5)cnc4c3)cc2)CC1 ; CN1CCN(c2ccc(-c3ccn4c(-c5ccnc6ccccc56)nnc4c3)cc2)CC1 ; c1ccc2c(-c3nnc4cc(-c5ccc(OCCN6CCOCC6)cc5)ccn34)ccnc2c1 ; CC(CN(C)C)Oc1ccc(-c2ccn3c(-c4ccnc5ccccc45)nnc3c2)cc1 ; c1ccc2c(-c3nnc4cc(-c5ccc(OCCN6CCCCC6)cc5)ccn34)ccnc2c1] 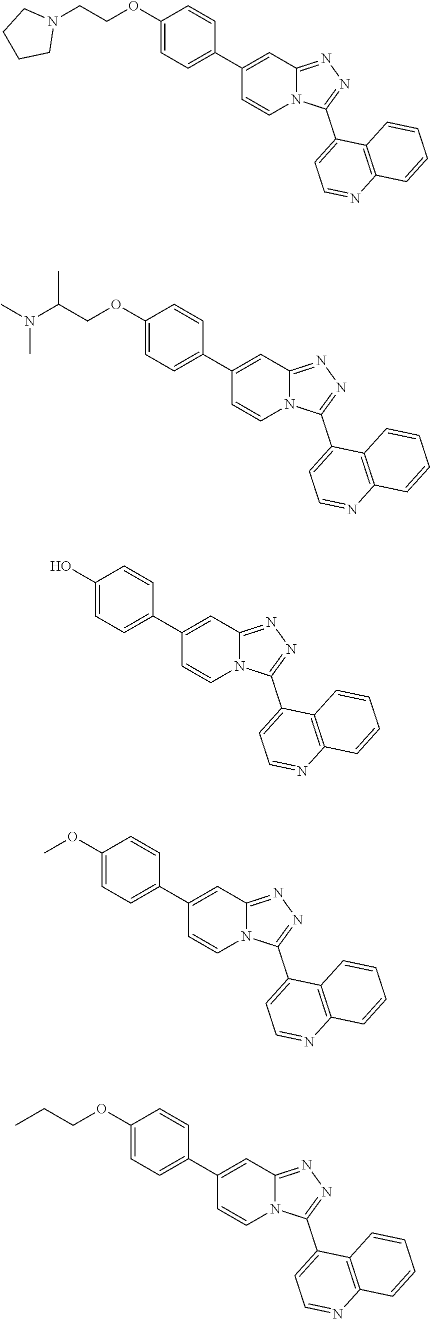 [SMILES: c1ccc2c(-c3nnc4cc(-c5ccc(OCCN6CCCC6)cc5)ccn34)ccnc2c1 ; CC(COc1ccc(-c2ccn3c(-c4ccnc5ccccc45)nnc3c2)cc1)N(C)C ; Oc1ccc(-c2ccn3c(-c4ccnc5ccccc45)nnc3c2)cc1 ; COc1ccc(-c2ccn3c(-c4ccnc5ccccc45)nnc3c2)cc1 ; CCCOc1ccc(-c2ccn3c(-c4ccnc5ccccc45)nnc3c2)cc1] 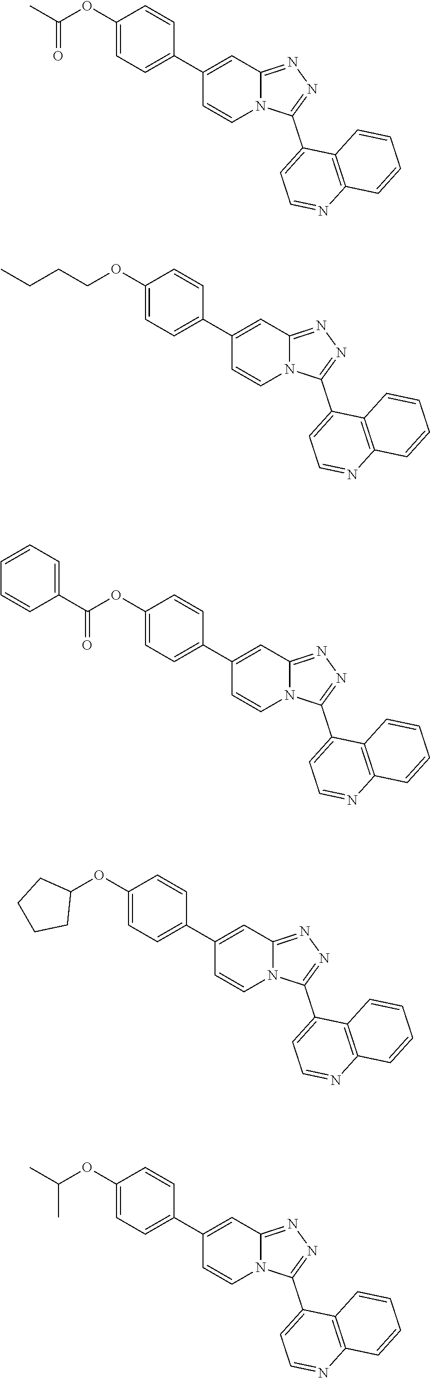 [SMILES: CC(=O)Oc1ccc(-c2ccn3c(-c4ccnc5ccccc45)nnc3c2)cc1 ; CCCCOc1ccc(-c2ccn3c(-c4ccnc5ccccc45)nnc3c2)cc1 ; O=C(Oc1ccc(-c2ccn3c(-c4ccnc5ccccc45)nnc3c2)cc1)c1ccccc1 ; c1ccc2c(-c3nnc4cc(-c5ccc(OC6CCCC6)cc5)ccn34)ccnc2c1 ; CC(C)Oc1ccc(-c2ccn3c(-c4ccnc5ccccc45)nnc3c2)cc1] 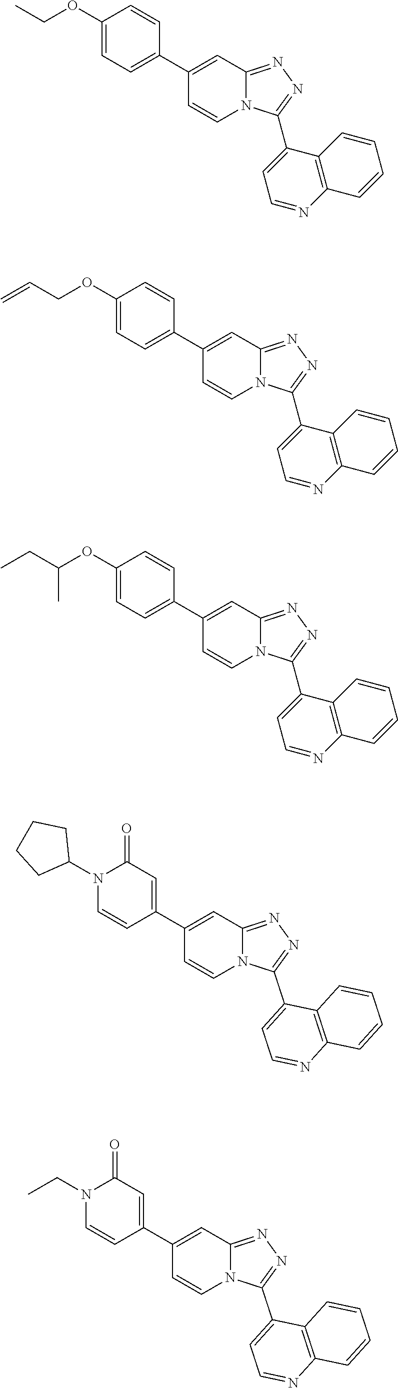 [SMILES: CCOc1ccc(-c2ccn3c(-c4ccnc5ccccc45)nnc3c2)cc1 ; C=CCOc1ccc(-c2ccn3c(-c4ccnc5ccccc45)nnc3c2)cc1 ; CCC(C)Oc1ccc(-c2ccn3c(-c4ccnc5ccccc45)nnc3c2)cc1 ; O=c1cc(-c2ccn3c(-c4ccnc5ccccc45)nnc3c2)ccn1C1CCCC1 ; CCn1ccc(-c2ccn3c(-c4ccnc5ccccc45)nnc3c2)cc1=O] 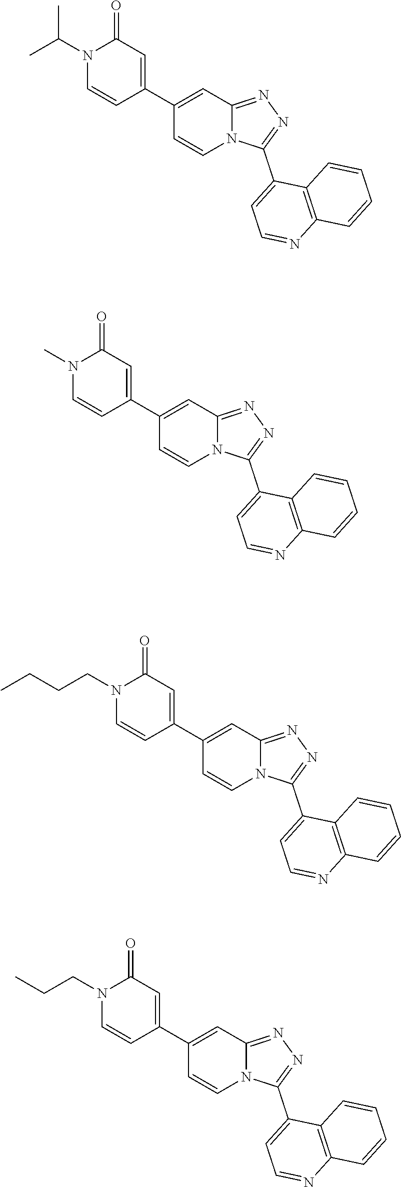 [SMILES: CC(C)n1ccc(-c2ccn3c(-c4ccnc5ccccc45)nnc3c2)cc1=O ; Cn1ccc(-c2ccn3c(-c4ccnc5ccccc45)nnc3c2)cc1=O ; CCCCn1ccc(-c2ccn3c(-c4ccnc5ccccc45)nnc3c2)cc1=O ; CCCn1ccc(-c2ccn3c(-c4ccnc5ccccc45)nnc3c2)cc1=O] 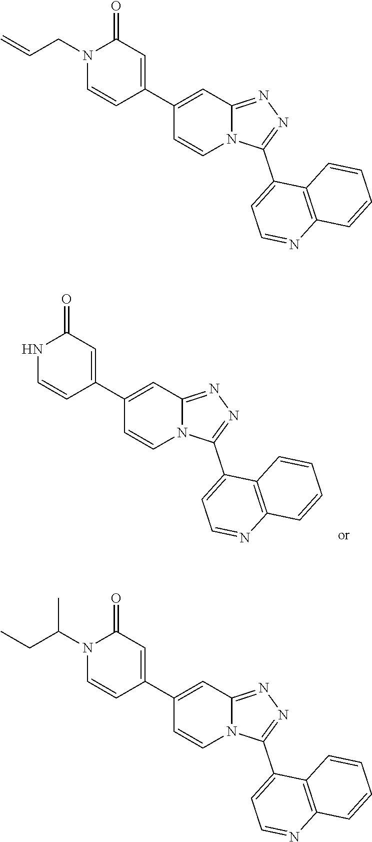 [SMILES: C=CCn1ccc(-c2ccn3c(-c4ccnc5ccccc45)nnc3c2)cc1=O ; O=c1cc(-c2ccn3c(-c4ccnc5ccccc45)nnc3c2)cc[nH]1 ; CCC(C)n1ccc(-c2ccn3c(-c4ccnc5ccccc45)nnc3c2)cc1=O]
or a pharmaceutically acceptable salt thereof.

13. A method of treating or preventing a disease or condition comprising administering to a subject a compound having the structure represented by Formula II: 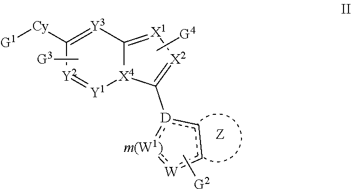
wherein:
X¹ is N or CR⁵;
X², and X⁴ are independently N or CR⁵;
Y¹, Y², and Y³ are independently N or CR⁵;
D is C or N;
W is N or O;
W₁ is N, O or C;
Cy is substituted with one or more R¹, and is selected from C₃-C₁₂ cycloalkyl, C₃-C₁₂ cycloalkenyl, aryl, heteroaryl, or C₃-C₁₂ heterocyclyl.
G¹-G⁴ are absent or independently selected from H, halogen, CN, CF₃, C₁₋₁₀ alkyl, C₃₋₁₀ cycloalkyl, OC₁₋₁₀ alkyl optionally substituted with a C₃₋₈ membered ring containing C, O, S or N, optionally substituted with one or more R⁶, or NR⁶C₁₋₁₀ alkyl optionally substituted with a C₃₋₈ membered ring containing C, O, S or N, optionally substituted with one or more R⁶;
R⁵ and R⁶ are independently selected from H, halogen, CN, CF₃, C₁₋₁₀ alkyl, C₃₋₁₀ cycloalkyl, or OC₁₋₁₀ alkyl optionally substituted with a C₃₋₈ membered ring containing C, O, S or N;
Z is optionally substituted with one or more R₅, and is selected from C₃-C₁₂ cycloalkyl, C₃-C₁₂ cycloalkenyl, aryl, heteroaryl, or C₃-C₁₂ heterocyclyl; and
m is 1 or 2.

14. The method of claim 13, where in D is C and m is 2.

15. The method of claim 13, where W is N

16. A method of treating or preventing a disease or condition comprising administering to a subject a compound having the structure represented by Formula III: 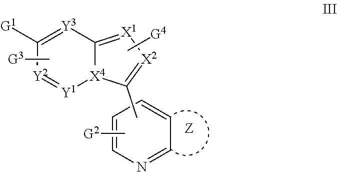
wherein:
X¹, X², and X⁴ are independently N or CR⁵;
Y¹, Y², and Y³ are independently N or CR⁵;
G¹-G⁵ are absent or independently selected from H, halogen, CN, CF₃, C₁₋₁₀ alkyl, C₃₋₁₀ cycloalkyl, or OC₁₋₁₀ alkyl optionally substituted with a C₃₋₈ membered ring containing C, O, S or N, optionally substituted with one or more R⁶;
R⁵ and R⁶ are independently selected from H, halogen, CN, CF₃, C₁₋₁₀ alkyl, C₃₋₁₀ cycloalkyl, or OC₁₋₁₀ alkyl optionally substituted with a C₃₋₈ membered ring containing C, O, S or N; and
Z is optionally substituted with one or more G⁵, and is selected from C₃-C₁₂ cycloalkyl, C₃-C₁₂ cycloalkenyl, aryl, heteroaryl, or C₃-C₁₂ heterocyclyl.

17. The method of claim 16, wherein the compound has the formula:

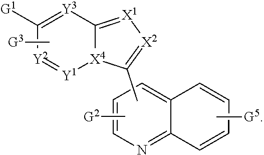

18. The method of claim 16 or 17, wherein
i. X¹ is N, X⁴ is N, and X², X⁴, Y², and Y³ are independently CR⁵;
ii. X¹ is N, X² is N, Y¹ is N, and X⁴, Y², and Y³ are independently CR⁵;
iii. X¹ is N, X² is N, Y² is N, and X⁴, Y², and Y³ are independently CR⁵; or
iv. X¹ is N, and X², X³, X⁴, Y¹, Y², and Y³ are independently CR⁵;
Y¹, Y², and Y³ are independently N or CR⁵.

19. The method of claim 18, wherein X¹ is N, X⁴ is N, and X², X⁴, Y¹, Y², and Y³ are CH.

20. The method of claim 16, wherein X¹, X⁴, and Y¹ are N, and X², X⁴, Y², and Y³ are CH.

21. The method of claim 16, wherein X¹, X⁴, and Y² are N, and X², X⁴, and Y³ are CH.

22. The method of claim 16, wherein G² is absent.

23. The method of claim 16, wherein G³ is absent.

24. The method of claim 1, wherein the disease is a cancer selected from colorectal cancer, sporadic colorectal cancer, acute myeloid leukemia, chronic myelogenous leukemia, non-small cell lung cancer (NSCLC), pancreatic cancer, ovarian cancer, serous ovarian cancer, epithelial ovarian cancer, melanoma, or head and neck squamous cell carcinoma (HNSCC).

25. The method of claim 1, wherein the disease is a cancer of the central nervous system.

26. The method of claim 25, wherein the cancer is a glioma, astrocytic glioma, diffuse intrinsic pontine glioma (DIPG), high grade glioma (HGG), germ cell tumor, glioblastoma multiform (GBM), oligodendroglioma, pituitary tumor, or ependymoma.

27. The method of claim 1, wherein the disease is iron-refractory iron-deficient anemia (IRIDA), heterotopic ossification, nonhereditary myositis ossificans, myositis ossificans traumatica, or myositis ossificans circumscripta.

28. The method of claim 1, wherein the compound is administered in a pharmaceutical composition with a pharmaceutically acceptable carrier.

29. The method of claim 1, wherein the compound is administered topically, orally, nasally, intravenously, intramuscularly, intraarterially, intracapsularly, intraorbitally, intracardiacly, intradermally, intraperitoneally, transmucosally, transdermally, anally, rectally, vaginally, transtracheally, subcutaneously, subcuticularly, intraarticularly, or subcapsularly.

30. The method of claim 29, wherein the compound is administered intravenously.

31. The method of claim 29, wherein the pharmaceutical composition is administered orally.
